SEQUENCE LISTING (142 sequences)

SEQ ID NO: 1 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, Homo sapiens
GIVEQCCTSICSLYQLENYCN

SEQ ID NO: 3 - protein, 30 aa, Homo sapiens
FVNQHLCGSHLVEALYLVCGERGFFYTPKT

SEQ ID NO: 4 - protein, 23 aa, Artificial Sequence
  insulin peptide analog
  MISC_FEATURE (1)..(1): Xaa at position 1 is any amino acid
  MISC_FEATURE (2)..(2): Xaa at position 2 is any amino acid or a modified amino acid comprising a glucose binding element linked to its side chain
  MISC_FEATURE (3)..(3): Xaa at position 3 is any amino acid or a modified amino acid comprising a glucose binding element linked to its side chain
  MISC_FEATURE (8)..(8): Xaa at position 8 is Cys or selenocysteine
  MISC_FEATURE (10)..(10): Xaa at position 10 is Thr, His, Lys, Arg, or Glu
  MISC_FEATURE (13)..(13): Xaa at position 13 is Cys or selenocysteine
XXXIVEQXCXSIXSLYQLENYCN

SEQ ID NO: 5 - protein, 22 aa, Artificial Sequence
  insulin peptide analog
  MISC_FEATURE (1)..(1): Xaa at position 1 is any amino acid or a modified amino acid comprising a glucose binding element linked to its side chain
  MISC_FEATURE (2)..(2): Xaa at position 2 is any amino acid or a modified amino acid comprising a glucose binding element linked to its side chain
  MISC_FEATURE (7)..(7): Xaa at position 7 is Cys or selenocysteine
  MISC_FEATURE (9)..(9): Xaa at position 9 is Thr, His, Lys, Arg, or Glu
  MISC_FEATURE (12)..(12): Xaa at position 12 is Cys or selenocysteine
XXIVEQXCXSIXSLYQLENYCN

SEQ ID NO: 6 - protein, 30 aa, Artificial Sequence
  insulin peptide analog
  MISC_FEATURE (3)..(3): Xaa at position 3 is Lys or Asn
  MISC_FEATURE (28)..(28): Xaa at position 28 is Orn, Glu Asp, Lys, Pro, or a modified amino acid comprising a diol bearing moiety linked to the side chain of the amino acid
  MISC_FEATURE (29)..(29): Xaa at position 29 is Orn, Lys, Pro, or a modified amino acid comprising a diol bearing moiety linked to the side chain of the amino acid
  MISC_FEATURE (30)..(30): Xaa at position 30 is Thr, or a diol bearing amino acid
FVXQHLCGSHLVEALYLVCGERGFFYTXXX

SEQ ID NO: 7 - protein, 28 aa, Artificial Sequence
  insulin peptide analog
  MISC_FEATURE (3)..(3): Xaa at position 3 is Lys or Asn
  MISC_FEATURE (28)..(28): Xaa at position 28 is a diol bearing amino acid
FVXQHLCGSHLVEALYLVCGERGFFYTX

SEQ ID NO: 8 - protein, 23 aa, Artificial Sequence
  insulin peptide analog
  MISC_FEATURE (2)..(2): Xaa at position 2 is Cys comprising a glucose binding element linked to its side chain
  MISC_FEATURE (8)..(8): Xaa at position 8 is selenocysteine
  MISC_FEATURE (10)..(10): Xaa at position 10 is His
  MISC_FEATURE (13)..(13): Xaa at position 13 is selenocysteine
GXGIVEQXCXSIXSLYQLENYCN

SEQ ID NO: 9 - protein, 22 aa, Artificial Sequence
  insulin peptide analog
FVNQHLCGSHLVEALYLVCGER

SEQ ID NO: 10 - protein, 8 aa, Artificial Sequence
  insulin peptide analog
  MISC_FEATURE (6)..(6): Xaa at position 6 is a modified Lys comprising a diol bearing moiety linked to the side chain of the amino acid
GFFYTXPT

SEQ ID NO: 11 - protein, 50 aa, Artificial Sequence
  insulin peptide analog
  MISC_FEATURE (30)..(30): Xaa at position 30 is D-Dap(Pr)
FVNQHLCGSHLVEALYLVCGERGFFYTKGXIVEQCCHSICSLYQLENYCN

SEQ ID NO: 12 - protein, 51 aa, Artificial Sequence
  insulin peptide analog
  MISC_FEATURE (30)..(30): Xaa at position 30 is D-Dap(Pr)
FVNQHLCGSHLVEALYLVCGERGFFYTKGXGIVEQCCHSICSLYQLENYCN

SEQ ID NO: 13 - protein, 23 aa, Artificial Sequence
  insulin peptide analog
  MISC_FEATURE (2)..(2): Xaa at position 2 is a modified D-Cys, L-Cys, D-homoCys, L-homoCys, D-Asp, L-Asp, D-Glu, L-Glu, L-Lys, D-Lys, L-Orn, D-Orn, L-Dab, D-Dap, and L-Dap,wherein the modified amino acid comprises a glucose-binding elements linked to the side chain of
  MISC_FEATURE (8)..(8): Xaa at position 8 is Cys or selenocysteine
  MISC_FEATURE (10)..(10): Xaa at position 10 is Thr, His, Lys, Arg, or Glu
  MISC_FEATURE (13)..(13): Xaa at position 13 is Cys or selenocysteine
  MISC_FEATURE (23)..(23): Xaa at position 23 is asparagine, serine, glycine or alanine
GXGIVEQXCXSIXSLYQLENYCX

SEQ ID NO: 14 - protein, 22 aa, Artificial Sequence
  insulin peptide analog
  MISC_FEATURE (1)..(1): Xaa at position 1 is a modified L-homoCys, D-homoCys, L-Asp, D-Asp, L-Glu, D-Glu, LDap, D-Dap, L-Cys, L-Lys, D-Lys, L-Orn, D-Orn, L-Dab, and D-Dab, wherein the modified amino acid comprises a glucose-binding elements linked to the side chain of
  MISC_FEATURE (7)..(7): Xaa at position 7 is Cys or selenocysteine
  MISC_FEATURE (9)..(9): Xaa at position 9 is Thr, His, Lys, Arg, or Glu
  MISC_FEATURE (12)..(12): Xaa at position 12 is Cys or selenocysteine
  MISC_FEATURE (22)..(22): Xaa at position 22 is asparagine, serine, glycine or alanine
XGIVEQXCXSIXSLYQLENYCN

SEQ ID NO: 15 - protein, 22 aa, Artificial Sequence
  insulin peptide analog
  MISC_FEATURE (2)..(2): Xaa at position 2 is a modified L-homoCys, D-homoCys, L-Asp, D-Asp, L-Glu, D-Glu, LDap, D-Dap, L-Cys, L-Lys, D-Lys, L-Orn, D-Orn, L-Dab, and D-Dab, wherein the modified amino acid comprises a glucose-binding elements linked to the side chain of
  MISC_FEATURE (7)..(7): Xaa at position 7 is Cys or selenocysteine
  MISC_FEATURE (9)..(9): Xaa at position 9 is Thr, His, Lys, Arg, or Glu
  MISC_FEATURE (12)..(12): Xaa at position 12 is Cys or selenocysteine
  MISC_FEATURE (22)..(22): Xaa at position 22 is asparagine, serine, glycine or alanine
GXIVEQXCXSIXSLYQLENYCX

SEQ ID NO: 16 - protein, 21 aa, Artificial Sequence
  insulin peptide analog
  MISC_FEATURE (1)..(1): Xaa at position 1 is a modified L-Cys, L-Lys, D-Lys, L-Orn, D-Orn, L-Dab, and D-Dab, wherein the modified amino acid comprises a glucose-binding elements linked to the side chain of the amino acid
  MISC_FEATURE (6)..(6): Xaa at position 6 is Cys or selenocysteine
  MISC_FEATURE (8)..(8): Xaa at position 8 is Thr, His, Lys, Arg, or Glu
  MISC_FEATURE (11)..(11): Xaa at position 11 is Cys or selenocysteine
XIVEQXCXSIXSLYQLENYCN

SEQ ID NO: 17 - protein, 25 aa, Artificial Sequence
  insulin peptide analog
  MISC_FEATURE (3)..(3): Xaa at position 3 is Lys or Asn
  MISC_FEATURE (5)..(5): Xaa at position 5 is histidine or threonine
  MISC_FEATURE (9)..(9): Xaa at position 9 is alanine, glycine or serine
  MISC_FEATURE (10)..(10): Xaa at position 10 is histidine, aspartic acid or glutamic acid
FVXQXLCGXXLVEALYLVCGERGFF

SEQ ID NO: 18 - protein, 5 aa, Artificial Sequence
  insulin peptide analog
  MISC_FEATURE (3)..(3): Xaa at position 3 is proline, aspartic acid or glutamic acid
YTXKT

SEQ ID NO: 19 - protein, 5 aa, Artificial Sequence
  insulin peptide analog
  MISC_FEATURE (3)..(3): Xaa at position 3 is proline, aspartic acid or glutamic acid
YTXKP

SEQ ID NO: 20 - protein, 5 aa, Artificial Sequence
  insulin peptide analog
YTKPT

SEQ ID NO: 21 - protein, 6 aa, Artificial Sequence
  insulin peptide analog
YTKPTR

SEQ ID NO: 22 - protein, 7 aa, Artificial Sequence
  insulin peptide analog
YTKPTRR

SEQ ID NO: 23 - protein, 4 aa, Artificial Sequence
  insulin peptide analog
  MISC_FEATURE (3)..(3): Xaa at position 3 is proline, aspartic acid or glutamic acid
YTXK

SEQ ID NO: 24 - protein, 4 aa, Artificial Sequence
  insulin peptide analog
YTKP

SEQ ID NO: 25 - protein, 4 aa, Artificial Sequence
  insulin peptide analog
YTPK

SEQ ID NO: 26 - protein, 30 aa, Artificial Sequence
  insulin peptide analog
  MISC_FEATURE (3)..(3): Xaa at position 3 is Lys or Asn
  MISC_FEATURE (28)..(28): Xaa at position 28 is proline, aspartic acid or glutamic acid
FVXQHLCGSHLVEALYLVCGERGFFYTXKP

SEQ ID NO: 27 - protein, 21 aa, Artificial Sequence
  insulin peptide analog
  MISC_FEATURE (8)..(8): Xaa at position 8 is of threonine lysine, arginine, glutamic acid, or histidine
  MISC_FEATURE (21)..(21): Xaa at position 21 is asparagine, glycine and alanine
GIVEQCCXSICSLYQLENYCX

SEQ ID NO: 28 - protein, 30 aa, Artificial Sequence
  insulin peptide analog
  MISC_FEATURE (5)..(5): Xaa at position 5 is histidine or threonine
FVNQXLCGSHLVEALYLVCGERGFFYTEKT

SEQ ID NO: 29 - protein, 30 aa, Artificial Sequence
  insulin peptide analog
  MISC_FEATURE (5)..(5): Xaa at position 5 is histidine or threonine
FVNQXLCGSHLVEALYLVCGERGFFYTDKT

SEQ ID NO: 30 - protein, 30 aa, Artificial Sequence
  insulin peptide analog
  MISC_FEATURE (5)..(5): Xaa at position 5 is histidine or threonine
FVNQXLCGSHLVEALYLVCGERGFFYTKPT

SEQ ID NO: 31 - protein, 30 aa, Artificial Sequence
  insulin peptide analog
  MISC_FEATURE (5)..(5): Xaa at position 5 is histidine or threonine
FVNQHLCGSHLVEALYLVCGERGFFYTKKP

SEQ ID NO: 32 - protein, 30 aa, Artificial Sequence
  insulin peptide analog
  MISC_FEATURE (5)..(5): Xaa at position 5 is histidine or threonine
FVNQXLCGSHLVEALYLVCGERGFFYTPKT

SEQ ID NO: 33 - DNA, 20 bp, Artificial Sequence
  insulin peptide analog
gttcaatgccaggttcccag

SEQ ID NO: 34 - DNA, 19 bp, Artificial Sequence
  insulin peptide analog
ttgcaggccagttgttgac

SEQ ID NO: 35 - DNA, 20 bp, Artificial Sequence
  insulin peptide analog
agagacaagatccgcctgaa

SEQ ID NO: 36 - DNA, 19 bp, Artificial Sequence
  insulin peptide analog
cttccagtagttccctcca

SEQ ID NO: 37 - DNA, 22 bp, Artificial Sequence
  insulin peptide analog
cgacatcgaagacatgcttcag

SEQ ID NO: 38 - DNA, 21 bp, Artificial Sequence
  insulin peptide analog
ggaaggcttcaagagaggagc

SEQ ID NO: 39 - DNA, 21 bp, Artificial Sequence
  insulin peptide analog
atggtttacatgttccaatat

SEQ ID NO: 40 - DNA, 22 bp, Artificial Sequence
  insulin peptide analog
atgaggtccaccaccctggttg

SEQ ID NO: 41 - protein, 8 aa, Artificial Sequence
  insulin peptide fragment
  MISC_FEATURE (6)..(6): Xaa at position 6 is Lys, Orn, Dab, or Dap with sidechain linked GBE 1-6
GFFYTXPT

SEQ ID NO: 42 - protein, 23 aa, Artificial Sequence
  insulin peptide analog
  MISC_FEATURE (2)..(2): Xaa at position 2 is cyteine with linked GBE
GXGIVEQCCTSICSLYQLENYCN

SEQ ID NO: 43 - protein, 30 aa, Artificial Sequence
  insulin peptide analog
  MISC_FEATURE (5)..(5): Xaa at position 5 is histidine or threonine
  MISC_FEATURE (28)..(28): Xaa at position 28 is a modfied lysine comprising DHBA
FVNQHLCGSHLVEALYLVCGERGFFYTXPT

SEQ ID NO: 44 - protein, 22 aa, Artificial Sequence
  insulin peptide analog
  MISC_FEATURE (2)..(2): Xaa at position 2 is dCys comprising a glucose binding element linked to its side chain
  MISC_FEATURE (7)..(7): Xaa at position 7 is selenocysteine
  MISC_FEATURE (12)..(12): Xaa at position 12 is selenocysteine
GXIVEQXCTSIXSLYQLENYCN

SEQ ID NO: 45 - protein, 23 aa, Artificial Sequence
  insulin peptide analog
  MISC_FEATURE (2)..(2): Xaa at position 2 is Cys comprising a glucose binding element linked to its side chain
  MISC_FEATURE (8)..(8): Xaa at position 8 is selenocysteine
  MISC_FEATURE (13)..(13): Xaa at position 13 is selenocysteine
GXGIVEQXCTSIXSLYQLENYCN

SEQ ID NO: 46 - protein, 23 aa, Artificial Sequence
  insulin peptide analog
  MISC_FEATURE (2)..(2): Xaa at position 2 is dCys comprising a glucose binding element linked to its side chain
GXGIVEQCCTSICSLYQLENYCN

SEQ ID NO: 47 - protein, 22 aa, Artificial Sequence
  insulin peptide analog
  MISC_FEATURE (2)..(2): Xaa at position 2 is dCys comprising a glucose binding element linked to its side chain
GXIVEQCCTSICSLYQLENYCN

SEQ ID NO: 48 - protein, 22 aa, Artificial Sequence
  insulin peptide analog
  MISC_FEATURE (2)..(2): Xaa at position 2 is dCys comprising a glucose binding element linked to its side chain
  MISC_FEATURE (7)..(7): Xaa at position 7 is selenocysteine
  MISC_FEATURE (12)..(12): Xaa at position 12 is selenocysteine
GXIVEQXCTSIXSLYQLENYCN

SEQ ID NO: 49 - protein, 30 aa, Homo sapiens; identical to SEQ ID NO: 3

SEQ ID NO: 50 - protein, 30 aa, Artificial Sequence; identical to SEQ ID NO: 43
  insulin peptide analog
  MISC_FEATURE (5)..(5): Xaa at position 5 is histidine or threonine
  MISC_FEATURE (28)..(28): Xaa at position 28 is a modfied lysine comprising DHBA

SEQ ID NO: 51 - protein, 23 aa, Artificial Sequence
  insulin peptide analog
  MISC_FEATURE (2)..(2): Xaa at position 2 is Cys comprising a glucose binding element linked to its side chain
  MISC_FEATURE (8)..(8): Xaa at position 8 is selenocysteine
  MISC_FEATURE (13)..(13): Xaa at position 13 is selenocysteine
GXGIVEQCCTSICSLYQLENYCN

SEQ ID NO: 52 - protein, 22 aa, Artificial Sequence
  insulin peptide analog
  misc_feature (1)..(1): Xaa can be any naturally occurring amino acid
  MISC_FEATURE (2)..(2): Xaa at position 2 is Cys comprising a glucose binding element linked to its side chain
XGIVEQCCTSICSLYQLENYCN

SEQ ID NO: 53 - protein, 22 aa, Artificial Sequence
  insulin peptide analog
  misc_feature (1)..(1): Xaa can be any naturally occurring amino acid
  MISC_FEATURE (2)..(2): Xaa at position 2 is Cys comprising a glucose binding element linked to its side chain
XGIVEQCCTSICSLYQLENYCN

SEQ ID NO: 54 - protein, 22 aa, Artificial Sequence
  insulin peptide analog
  misc_feature (1)..(1): Xaa can be any naturally occurring amino acid
  MISC_FEATURE (2)..(2): Xaa at position 2 is dCys comprising a glucose binding element linked to its side chain
XGIVEQCCTSICSLYQLENYCN

SEQ ID NO: 55 - protein, 22 aa, Artificial Sequence
  insulin peptide analog
  misc_feature (1)..(1): Xaa can be any naturally occurring amino acid
  MISC_FEATURE (2)..(2): Xaa at position 2 is dCys comprising a glucose binding element linked to its side chain
XGIVEQCCTSICSLYQLENYCN

SEQ ID NO: 56 - protein, 23 aa, Artificial Sequence
  insulin peptide analog
  MISC_FEATURE (2)..(2): Xaa at position 2 is Dap comprising a glucose binding element linked to its side chain
GXGIVEQCCTSICSLYQLENYCN

SEQ ID NO: 57 - protein, 22 aa, Artificial Sequence
  insulin peptide analog
  MISC_FEATURE (2)..(2): Xaa at position 2 is Dap comprising a glucose binding element linked to its side chain
GXIVEQCCHSICSLYQLENYCN

SEQ ID NO: 58 - protein, 22 aa, Artificial Sequence
  insulin peptide analog
  MISC_FEATURE (2)..(2): Xaa at position 2 is dDap comprising a glucose binding element linked to its side chain
GXIVEQCCHSICSLYQLENYCN

SEQ ID NO: 59 - protein, 22 aa, Artificial Sequence
  insulin peptide analog
  MISC_FEATURE (2)..(2): Xaa at position 2 is dDap comprising a glucose binding element linked to its side chain
GXIVEQCCTSICSLYQLENYCN

SEQ ID NO: 60 - protein, 22 aa, Artificial Sequence
  insulin peptide analog
  MISC_FEATURE (2)..(2): Xaa at position 2 is dOrn comprising a glucose binding element linked to its side chain
GXIVEQCCHSICSLYQLENYCN

SEQ ID NO: 61 - protein, 22 aa, Artificial Sequence
  insulin peptide analog
  MISC_FEATURE (2)..(2): Xaa at position 2 is dLys comprising a glucose binding element linked to its side chain
GXIVEQCCHSICSLYQLENYCN

SEQ ID NO: 62 - protein, 30 aa, Homo sapiens
  MISC_FEATURE (30)..(30): Xaa at position 30 is a diol bearing amino acid derivative
FVKQHLCGSHLVEALYLVCGERGFFYTEKX

SEQ ID NO: 63 - protein, 30 aa, Homo sapiens
  MISC_FEATURE (30)..(30): Xaa at position 30 is a diol bearing amino acid derivative
FVNQHLCGSHLVEALYLVCGERGFFYTDKX

SEQ ID NO: 64 - protein, 30 aa, Homo sapiens
  MISC_FEATURE (30)..(30): Xaa at position 30 is a diol bearing amino acid derivative
FVNQHLCGSHLVEALYLVCGERGFFYTKPX

SEQ ID NO: 65 - protein, 30 aa, Homo sapiens
  MISC_FEATURE (30)..(30): Xaa at position 30 is a diol bearing amino acid derivative
FVNQHLCGSHLVEALYLVCGERGFFYTPKX

SEQ ID NO: 66 - protein, 29 aa, Homo sapiens
  MISC_FEATURE (29)..(29): Xaa at position 29 is a diol bearing amino acid derivative
FVNQHLCGSHLVEALYLVCGERGFFYTKX

SEQ ID NO: 67 - protein, 30 aa, Homo sapiens
  MISC_FEATURE (29)..(29): Xaa at position 29 is ornithine
  MISC_FEATURE (30)..(30): Xaa at position 30 is a diol bearing amino acid derivative
FVNQHLCGSHLVEALYLVCGERGFFYTPXX

SEQ ID NO: 68 - protein, 29 aa, Homo sapiens
  MISC_FEATURE (29)..(29): Xaa at position 29 is a diol bearing amino acid derivative
FVNQHLCGSHLVEALYLVCGERGFFYTPX

SEQ ID NO: 69 - protein, 29 aa, Homo sapiens
  MISC_FEATURE (28)..(28): Xaa at position 28 is ornithine
  MISC_FEATURE (29)..(29): Xaa at position 29 is a diol bearing amino acid derivative
FVNQHLCGSHLVEALYLVCGERGFFYTXX

SEQ ID NO: 70 - protein, 28 aa, Homo sapiens
  MISC_FEATURE (28)..(28): Xaa at position 28 is a diol bearing amino acid derivative
FVNQHLCGSHLVEALYLVCGERGFFYTX

SEQ ID NO: 71 - protein, 27 aa, Homo sapiens
  MISC_FEATURE (27)..(27): Xaa at position 27 is a diol bearing amino acid derivative
FVNQHLCGSHLVEALYLVCGERGFFYX

SEQ ID NO: 72 - protein, 27 aa, Homo sapiens
  MISC_FEATURE (26)..(26): Xaa at position 26 is sarcosine
  MISC_FEATURE (27)..(27): Xaa at position 27 is ADP
FVNQHLCGSHLVEALYLVCGERGFFXX

SEQ ID NO: 73 - protein, 27 aa, Homo sapiens
  MISC_FEATURE (26)..(26): Xaa at position 26 is dAla
  MISC_FEATURE (27)..(27): Xaa at position 27 is ADP
FVNQHLCGSHLVEALYLVCGERGFFXX

SEQ ID NO: 74 - protein, 28 aa, Homo sapiens
  MISC_FEATURE (28)..(28): Xaa at position 28 is ADP
FVNQHLCGSHLVEALYLVCGERGFFYGX

SEQ ID NO: 75 - protein, 31 aa, Homo sapiens
  misc_feature (30)..(30): Xaa can be any naturally occurring amino acid
  MISC_FEATURE (31)..(31): Xaa at position 31 is a diol bearing amino acid derivative
FVNQHLCGSHLVEALYLVCGERGFFYTDKXX

SEQ ID NO: 76 - protein, 31 aa, Homo sapiens
  misc_feature (30)..(30): Xaa can be any naturally occurring amino acid
  MISC_FEATURE (31)..(31): Xaa at position 31 is a diol bearing amino acid derivative
FVNQHLCGSHLVEALYLVCGERGFFYTKPXX

SEQ ID NO: 77 - protein, 31 aa, Homo sapiens
  misc_feature (30)..(30): Xaa can be any naturally occurring amino acid
  MISC_FEATURE (31)..(31): Xaa at position 31 is a diol bearing amino acid derivative
FVNQHLCGSHLVEALYLVCGERGFFYTPKXX

SEQ ID NO: 78 - protein, 32 aa, Homo sapiens
  misc_feature (30)..(31): Xaa can be any naturally occurring amino acid
  MISC_FEATURE (32)..(32): Xaa at position 32 is a diol bearing amino acid derivative
FVNQHLCGSHLVEALYLVCGERGFFYTDKXXX

SEQ ID NO: 79 - protein, 32 aa, Homo sapiens
  misc_feature (30)..(31): Xaa can be any naturally occurring amino acid
  MISC_FEATURE (32)..(32): Xaa at position 32 is a diol bearing amino acid derivative
FVNQHLCGSHLVEALYLVCGERGFFYTKPXXX

SEQ ID NO: 80 - protein, 32 aa, Homo sapiens
  misc_feature (30)..(31): Xaa can be any naturally occurring amino acid
  MISC_FEATURE (32)..(32): Xaa at position 32 is a diol bearing amino acid derivative
FVNQHLCGSHLVEALYLVCGERGFFYTPKXXX

SEQ ID NO: 81 - protein, 21 aa, Homo sapiens
GIVEQCCTSICSLYQLENYCN

SEQ ID NO: 82 - protein, 21 aa, Homo sapiens
GIVEQCCHSICSLYQLENYCN

SEQ ID NO: 83 - protein, 21 aa, Artificial Sequence
  insulin peptide analog
  MISC_FEATURE (1)..(1): Xaa at position 1 is D-amino acid comprising a glucose-binding element linked to its side chain
  MISC_FEATURE (6)..(6): Xaa at position 6 is cys or selenocysteine
  MISC_FEATURE (8)..(8): Xaa at position 8 is Thr, His, Lys, Arg, or Glu
  MISC_FEATURE (11)..(11): Xaa at position 11 is  cys or selenocysteine
XIVEQXCTSIXSLYQLENYCN

SEQ ID NO: 84 - protein, 51 aa, Homo sapiens
  MISC_FEATURE (30)..(30): Xaa at Position 30 is Cys
FVNQHLCGSHLVEALYLVCGERGFFYTKGXGIVEQCCTSICSLYQLENYCN

SEQ ID NO: 85 - protein, 51 aa, Homo sapiens; identical to SEQ ID NO: 84
  MISC_FEATURE (30)..(30): Xaa at Position 30 is Cys(GBE)

SEQ ID NO: 86 - protein, 22 aa, Homo sapiens
FVNQHLCGSHLVEALYLVCGER

SEQ ID NO: 87 - protein, 23 aa, Homo sapiens
FVNQHLCGSHLVEALYLVCGERG

SEQ ID NO: 88 - protein, 30 aa, Homo sapiens
FVNQHLCGSHLVEALYLVCGERGFFYTKPT

SEQ ID NO: 89 - protein, 30 aa, Homo sapiens; identical to SEQ ID NO: 43
  MISC_FEATURE (28)..(28): Xaa at position 28 is Lys(DHBA)

SEQ ID NO: 90 - protein, 28 aa, Homo sapiens
FVNQHLCGSHLVEALYLVCGERGFFYTK

SEQ ID NO: 91 - protein, 30 aa, Homo sapiens; identical to SEQ ID NO: 43
  MISC_FEATURE (28)..(28): Xaa at position 28 is Orn(DHBA)

SEQ ID NO: 92 - protein, 23 aa, Artificial Sequence
  insulin peptide analog
  MISC_FEATURE (2)..(2): Xaa at position 2 is Cys(GBE)
GXGIVEQCCTSICSLYQLENYCN

SEQ ID NO: 93 - protein, 22 aa, Artificial Sequence
  insulin peptide analog
  MISC_FEATURE (2)..(2): Xaa at position 2 is Cys(GBE)
GXIVEQCCTSICSLYQLENYCN

SEQ ID NO: 94 - protein, 23 aa, Artificial Sequence
  insulin peptide analog
  MISC_FEATURE (2)..(2): Xaa at position 2 is Cys(GBE)
  MISC_FEATURE (8)..(8): Xaa at position 8 is selenocysteine
  MISC_FEATURE (13)..(13): Xaa at position 13 is selenocysteine
GXGIVEQXCTSIXSLYQLENYCN

SEQ ID NO: 95 - protein, 22 aa, Artificial Sequence
  insulin peptide analog
  MISC_FEATURE (2)..(2): Xaa at position 2 is dCys(GBE)
  MISC_FEATURE (7)..(7): Xaa at position 7 is selenocysteine
  MISC_FEATURE (12)..(12): Xaa at position 12 is selenocysteine
GXIVEQXCTSIXSLYQLENYCN

SEQ ID NO: 96 - protein, 22 aa, Artificial Sequence
  insulin peptide analog
  MISC_FEATURE (1)..(1): Xaa at position 1 is Cys(GBE)
XGIVEQCCTSICSLYQLENYCN

SEQ ID NO: 97 - protein, 22 aa, Artificial Sequence
  insulin peptide analog
  MISC_FEATURE (2)..(2): Xaa at position 2 is dCys(GBE)
GXIVEQCCHSICSLYQLENYCN

SEQ ID NO: 98 - protein, 22 aa, Artificial Sequence
  insulin peptide analog
  MISC_FEATURE (2)..(2): Xaa at position 2 is D-Dap(Mtt)
GXIVEQCCHSICSLYQLENYCN

SEQ ID NO: 99 - protein, 22 aa, Artificial Sequence
  insulin peptide analog
  MISC_FEATURE (2)..(2): Xaa at position 2 is D-Dap(Fmoc-Dap(Fmoc))
GXIVEQCCHSICSLYQLENYCN

SEQ ID NO: 100 - protein, 22 aa, Artificial Sequence
  insulin peptide analog
  MISC_FEATURE (2)..(2): Xaa at position 2 is D-Dap(H2N-Dap(NH2))
GXIVEQCCHSICSLYQLENYCN

SEQ ID NO: 101 - protein, 22 aa, Artificial Sequence
  insulin peptide analog
  MISC_FEATURE (2)..(2): Xaa at position 2 is D-Dap(BCM2-Dap(BCM2))
GXIVEQCCHSICSLYQLENYCN

SEQ ID NO: 102 - protein, 22 aa, Artificial Sequence
  insulin peptide analog
  MISC_FEATURE (2)..(2): Xaa at position 2 is D-Dap(NH2)
GXIVEQCCHSICSLYQLENYCN

SEQ ID NO: 103 - protein, 22 aa, Artificial Sequence
  insulin peptide analog
  MISC_FEATURE (2)..(2): Xaa at position 2 is D-Dap(Fmoc-Dap(Mtt))
GXIVEQCCHSICSLYQLENYCN

SEQ ID NO: 104 - protein, 22 aa, Artificial Sequence
  insulin peptide analog
  MISC_FEATURE (2)..(2): Xaa at position 2 is D-Dap(H2N-Dap(Mtt))
GXIVEQCCHSICSLYQLENYCN

SEQ ID NO: 105 - protein, 22 aa, Artificial Sequence
  insulin peptide analog
  MISC_FEATURE (2)..(2): Xaa at position 2 is D-Dap(Gly-Dap(NH2))
GXIVEQCCHSICSLYQLENYCN

SEQ ID NO: 106 - protein, 22 aa, Artificial Sequence
  insulin peptide analog
  MISC_FEATURE (2)..(2): Xaa at position 2 is D-Dap(H2N-Dap(FmocG))
GXIVEQCCHSICSLYQLENYCN

SEQ ID NO: 107 - protein, 22 aa, Artificial Sequence
  insulin peptide analog
  MISC_FEATURE (2)..(2): Xaa at position 2 is D-Dap(BCM2-Gly-Dap(BCM2))
GXIVEQCCHSICSLYQLENYCN

SEQ ID NO: 108 - protein, 22 aa, Artificial Sequence
  insulin peptide analog
  MISC_FEATURE (2)..(2): Xaa at position 2 is D-Dap(GBE-Gly-Dap(GBE))
GXIVEQCCHSICSLYQLENYCN

SEQ ID NO: 109 - protein, 22 aa, Artificial Sequence
  insulin peptide analog
  MISC_FEATURE (2)..(2): Xaa at position 2 is Dap(alloc)
GXIVEQCCHSICSLYQLENYCN

SEQ ID NO: 110 - protein, 23 aa, Homo sapiens
  MISC_FEATURE (2)..(2): Xaa at position 2 is dCys
GXGIVEQCCTSICSLYQLENYCN

SEQ ID NO: 111 - protein, 23 aa, Artificial Sequence
  insulin peptide analog
  MISC_FEATURE (2)..(2): Xaa at position 2 is dCys
  MISC_FEATURE (8)..(8): Xaa at position 8 is selenocysteine
  MISC_FEATURE (13)..(13): Xaa at position 13 is selenocysteine
GXGIVEQXCTSIXSLYQLENYCN

SEQ ID NO: 112 - protein, 22 aa, Artificial Sequence
  insulin peptide analog
  MISC_FEATURE (2)..(2): Xaa at position 2 is dCys
  MISC_FEATURE (7)..(7): Xaa at position 7 is selenocysteine
  MISC_FEATURE (12)..(12): Xaa at position 12 is selenocysteine
GXIVEQXCTSIXSLYQLENYCN

SEQ ID NO: 113 - protein, 22 aa, Artificial Sequence
  insulin peptide analog
  MISC_FEATURE (1)..(1): Xaa at position 1 is Cys(Acm)
XGIVEQCCHSICSLYQLENYCN

SEQ ID NO: 114 - protein, 23 aa, Artificial Sequence
  insulin peptide analog
  MISC_FEATURE (2)..(2): Xaa at position 2 is Cys(Npys)
  MISC_FEATURE (8)..(8): Xaa at position 8 is selenocysteine
  MISC_FEATURE (13)..(13): Xaa at position 13 is selenocysteine
GXGIVEQXCTSIXSLYQLENYCN

SEQ ID NO: 115 - protein, 23 aa, Artificial Sequence
  insulin peptide analog
  MISC_FEATURE (2)..(2): Xaa at position 2 is Cys
  MISC_FEATURE (8)..(8): Xaa at position 8 is selenocysteine
  MISC_FEATURE (13)..(13): Xaa at position 13 is selenocysteine
GXGIVEQXCTSIXSLYQLENYCN

SEQ ID NO: 116 - protein, 23 aa, Artificial Sequence
  insulin peptide analog
  MISC_FEATURE (2)..(2): Xaa at position 2 is dCys(Npys)
  MISC_FEATURE (8)..(8): Xaa at position 8 is selenocysteine
  MISC_FEATURE (13)..(13): Xaa at position 13 is selenocysteine
GXGIVEQXCTSIXSLYQLENYCN

SEQ ID NO: 117 - protein, 23 aa, Artificial Sequence
  insulin peptide analog
  MISC_FEATURE (2)..(2): Xaa at position 2 is dCys
  MISC_FEATURE (8)..(8): Xaa at position 8 is selenocysteine
  MISC_FEATURE (13)..(13): Xaa at position 13 is selenocysteine
GXGIVEQXCTSIXSLYQLENYCN

SEQ ID NO: 118 - protein, 23 aa, Artificial Sequence
  insulin peptide analog
  MISC_FEATURE (2)..(2): Xaa at position 2 is dCys(Acm)
  MISC_FEATURE (8)..(8): Xaa at position 8 is selenocysteine
  MISC_FEATURE (13)..(13): Xaa at position 13 is selenocysteine
GXGIVEQXCTSIXSLYQLENYCN

SEQ ID NO: 119 - protein, 22 aa, Artificial Sequence
  insulin peptide analog
  MISC_FEATURE (2)..(2): Xaa at position 2 is dCys(GBE)
  MISC_FEATURE (7)..(7): Xaa at position 7 is selenocysteine
  MISC_FEATURE (12)..(12): Xaa at position 12 is selenocysteine
GXIVEQXCTSIXSLYQLENYCN

SEQ ID NO: 120 - protein, 22 aa, Artificial Sequence
  insulin peptide analog
  MISC_FEATURE (2)..(2): Xaa at position 2 is Dap(GBE)
GXIVEQCCHSICSLYQLENYCN

SEQ ID NO: 121 - protein, 22 aa, Artificial Sequence
  insulin peptide analog
  MISC_FEATURE (2)..(2): Xaa at position 2 is Dap(GBE)
GXIVEQCCHSICSLYQLENYCN

SEQ ID NO: 122 - protein, 22 aa, Artificial Sequence
  insulin peptide analog
  MISC_FEATURE (2)..(2): Xaa at position 2 is DOrn(GBE)
GXIVEQCCHSICSLYQLENYCN

SEQ ID NO: 123 - protein, 23 aa, Artificial Sequence
  insulin peptide analog
  MISC_FEATURE (2)..(2): Xaa at position 2 is Orn(GBE)
GXGIVEQCCHSICSLYQLENYCN

SEQ ID NO: 124 - protein, 30 aa, Homo sapiens; identical to SEQ ID NO: 43
  MISC_FEATURE (28)..(28): Xaa at position 28 is Dap(GBE)

SEQ ID NO: 125 - protein, 22 aa, Artificial Sequence
  insulin peptide analog
  MISC_FEATURE (2)..(2): Xaa at position 2 is DDap(GBE)
GXIVEQCCTSICSLYQLENYCN

SEQ ID NO: 126 - protein, 28 aa, Homo sapiens
  MISC_FEATURE (27)..(27): Xaa at position 27 is Gly[APD]
  MISC_FEATURE (28)..(28): Xaa at position 28 is Gly[APD]
FVNQHLCGSHLVEALYLVCGERGFFYXX

SEQ ID NO: 127 - protein, 30 aa, Homo sapiens; identical to SEQ ID NO: 43
  MISC_FEATURE (28)..(28): Xaa at position 28 is K(GBE1-CAc)

SEQ ID NO: 128 - protein, 30 aa, Homo sapiens; identical to SEQ ID NO: 43
  MISC_FEATURE (28)..(28): Xaa at position 28 is Orn(GBE)

SEQ ID NO: 129 - protein, 22 aa, Artificial Sequence
  insulin peptide analog
  MISC_FEATURE (2)..(2): Xaa at position 2 is dDap(DHBA)
GXIVEQCCTSICSLYQLENYCN

SEQ ID NO: 130 - protein, 23 aa, Artificial Sequence
  insulin peptide analog
  MISC_FEATURE (2)..(2): Xaa at position 2 is Dap(DHBA)
GXGIVEQCCTSICSLYQLENYCN

SEQ ID NO: 131 - protein, 23 aa, Artificial Sequence
  insulin peptide analog
  MISC_FEATURE (2)..(2): Xaa at position 2 is Orn(DHBA)
GXGIVEQCCTSICSLYQLENYCN

SEQ ID NO: 132 - protein, 22 aa, Artificial Sequence
  insulin peptide analog
  MISC_FEATURE (2)..(2): Xaa at position 2 is dOrn(DHBA)
GXIVEQCCTSICSLYQLENYCN

SEQ ID NO: 133 - protein, 23 aa, Artificial Sequence
  insulin peptide analog
  MISC_FEATURE (2)..(2): Xaa at position 2 is DOPA
GXGIVEQCCTSICSLYQLENYCN

SEQ ID NO: 134 - protein, 23 aa, Artificial Sequence
  insulin peptide analog
  MISC_FEATURE (1)..(1): Xaa at position 1 is N(APD)
  MISC_FEATURE (2)..(2): Xaa at position 2 is DOPA
XXGIVEQCCTSICSLYQLENYCN

SEQ ID NO: 135 - protein, 23 aa, Artificial Sequence
  insulin peptide analog
  MISC_FEATURE (1)..(1): Xaa at position 1 is DOPA
  MISC_FEATURE (2)..(2): Xaa at position 2 is N(APD)
XXGIVEQCCTSICSLYQLENYCN

SEQ ID NO: 136 - protein, 23 aa, Artificial Sequence
  insulin peptide analog
  MISC_FEATURE (2)..(2): Xaa at position 2 is Dab(GBE)
GXGIVEQCCTSICSLYQLENYCN

SEQ ID NO: 137 - protein, 23 aa, Artificial Sequence
  insulin peptide analog
  MISC_FEATURE (2)..(2): Xaa at position 2 is Dap(DHBA)
GXGIVEQCCTSICSLYQLENYCN

SEQ ID NO: 138 - protein, 23 aa, Artificial Sequence
  insulin peptide analog
  MISC_FEATURE (2)..(2): Xaa at position 2 is Dap(DHBA)
  MISC_FEATURE (2)..(2): Xaa at position 2 is Orn(DHBA)
GXGIVEQCCTSICSLYQLENYCN

SEQ ID NO: 139 - protein, 23 aa, Artificial Sequence
  insulin peptide analog
  MISC_FEATURE (2)..(2): Xaa at position 2 is D-Orn(DHBA)
GXGIVEQCCTSICSLYQLENYCN

SEQ ID NO: 140 - protein, 23 aa, Artificial Sequence
  insulin peptide analog
  MISC_FEATURE (2)..(2): Xaa at position 2 is dDap(alloc)
GXGIVEQCCTSICSLYQLENYCN

SEQ ID NO: 141 - protein, 23 aa, Artificial Sequence
  Description of Artificial Sequence Synthetic peptide
  MOD_RES (2)..(2): Dap(alloc)
GXGIVEQCCTSICSLYQLENYCN

SEQ ID NO: 142 - protein, 22 aa, Artificial Sequence
  Description of Artificial Sequence Synthetic peptide
  MOD_RES (2)..(2): dDap(alloc)
GXIVEQCCTSICSLYQLENYCN

(19) United States
(12) Patent Application Publication
(10) Pub. No.: US 2024/0218041 A1
(43) Pub. Date: Jul. 4, 2024

(54) MOLECULAR DESIGN OF GLUCOSE SENSORS IN GLUCOSE-RESPONSIVE INSULIN ANALOGUES

(71) Applicant: THE TRUSTEES OF INDIANA UNIVERSITY, Bloomington, IN (US)

(72) Inventors: Michael A. WEISS, Indianapolis, IN (US); Mark A. JAROSINSKI, Fishers, IN (US)

(21) Appl. No.: 18/288,925

(22) PCT Filed: May 3, 2022
(86) PCT No.: PCT/US2022/027504

§ 371 Date: Oct. 30, 2023

Related U.S. Application Data
(60) Provisional application No. 63/185,325, filed on May 6, 2021.

(51) Int. Cl. C07K 14/62; C07K 1/107; C12P 21/02

(52) U.S. Cl. CPC C07K 14/622; C07K 1/1077; C12P 21/02

(57) ABSTRACT

A two-chain insulin analogue is provided containing (a) a B chain modified by the addition of a C-terminal diol element in combination with (b) a glucose-binding element attached to the A chain at or near its N terminus, optionally linked to a D-amino acid. A “flipped” set of insulin analogues wherein the A chain is modified by addition of an N-terminal diol element whereas the glucose-binding element is attached at or near the C terminus of the B chain is also provided. Compositions comprising such insulin analogues are used in methods of treating a patient with diabetes mellitus.

### CROSS REFERENCE TO RELATED APPLICATIONS

This application is a U.S. national counterpart application of international application serial No. PCT/US2022/027504 filed May 3, 2022, which claims priority to U.S. Provisional Patent Application No. 63/183,325 filed on May 3, 2021, the disclosures of which are hereby expressly incorporated herein by reference in their entireties.

### GOVERNMENT RIGHTS

This invention was made with government support under DK127761 and DK040949 awarded by the National Institutes of Health. The government has certain rights in the invention.

### INCORPORATION BY REFERENCE OF MATERIAL SUBMITTED ELECTRONICALLY

Incorporated by reference in its entirety is a computer-readable nucleotide/amino-acid sequence listing submitted concurrently herewith and identified as follows: 65 kilobytes ACII (text) file named “354919_ST25.txt,” created on May 3, 2022.

### BACKGROUND

The engineering of non-standard proteins, including therapeutic agents and vaccines, may have broad medical and societal benefits. Naturally occurring proteins—as encoded in the genomes of human beings, other mammals, vertebrate organisms, invertebrate organisms, or eukaryotic cells in general—may have evolved to function optimally within a cellular context but may be suboptimal for therapeutic applications. Analogues of such proteins may exhibit improved biophysical, biochemical, or biological properties. A benefit of protein analogues would be to achieve enhanced activity (such as metabolic regulation of metabolism leading to reduction in blood-glucose concentration under conditions of hyperglycemia) with decreased unfavorable effects (such as induction of hypoglycemia or its exacerbation).

An example of a therapeutic protein is provided by insulin. Wild-type human insulin and insulin molecules encoded in the genomes of other mammals bind to insulin receptors in multiple organs and diverse types of cells, irrespective of the receptor isoform generated by alternative modes of RNA splicing or by alternative patterns of post-translational glycosylation. An example of a medical benefit would be the non-standard design of a soluble insulin analogue whose intrinsic affinity for insulin receptors on the surface of target cells, and hence whose biological potency, would depend on the concentration of glucose in the blood stream. Such an analogue may have a three-dimensional conformation that changes as a function of glucose concentration and/or may have a covalent bond to an inhibitory molecular entity that is detached at high glucose concentrations. Although it is not presently known in the art how to engineer such hypothetical analogues, this long-sought class of protein analogues or protein derivatives is collectively designated “glucose-responsive insulins” (GRIs).

The insulin molecule contains two chains, an A chain, containing 21 residues, and a B chain containing 30 residues. The mature hormone is derived from a longer single-chain precursor, designated proinsulin, as outlined in FIG. 1A. FIG. 1B depicts a structural model of proinsulin, consisting of an insulin-like moiety and a disordered connecting peptide (C-domain). While, FIG. 1C is a schematic representation of the sequence of human insulin including the A-chain and the B-chain disulfide connectivity. Specific residues in the insulin molecule are indicated by the amino-acid type (typically in standard three-letter code; e.g., Lys and Ala indicate Lysine and Alanine) and in superscript the chain (A or B) and position in that chain; or as single-letter code; e.g., A, C, D, E, F, G, H, I, K, L, M, N, P, Q, R, S T, V, W, and Y indicate Alanine, Cysteine, Aspartic Acid, Glutamic Acid, Phenylalanine, Glycine, Histidine, Isoleucine, Lysine, Methionine, Asparagine, Proline, Arginine, Serine, Threonine, Valine, Tryptophan, and Tyrosine respectively For example, Alanine at position 14 of the B chain of human insulin is indicated by AlaB14 or A B14, and likewise Lysine at position B28 of insulin lispro (the active component of Humalog®; Eli Lilly and Co.) is indicated by LysB28 or K B28. Amino acids (except glycine) are chiral, and the configurations are designated L or D; L is presumed unless otherwise stated. D-Amino acids are designated “D-Cys” or “D-Ala” (and so forth) in three-letter code; the small capital “D” (D) is used to avoid ambiguity with “D” as code for Asp. Alternatively D-amino acids are designated “dC” or “dA” (and so forth) in one-letter code, generally within brackets in sequence string.

Although the insulin hormone is stored in the pancreatic β-cell as a Zn²⁺-stabilized hexamer, it functions as a Zn²⁺-free monomer in the bloodstream. The three-dimensional structure of an insulin monomer is shown as a ribbon model in FIG. 2. Pertinent to the logic of the present invention is the proximity of the C terminus of the B chain (B30) to the N terminus of the A chain (A1), often engaged in a salt bridge (FIG. 3A). Covalent tethering of the B-chain C-terminus (either from B30 or from neighboring residues B28, B29, B31 or B32) to A-chain B terminus blocks binding of the hormone to the insulin receptor (FIG. 3B); such tethers block a conformational switch on receptor engagement. Inserting four or more residues into a foreshortened C domain restores receptor binding and agonist activity. Thus it is herein recognized that control of conformation(s) of the C-terminal B chain in its closed (inactive) and open (active) forms by use of reversible covalent bonding, provides the opportunity for ligand-controlled glucose responsivity (FIG. 3C; Chen, Y. S., et al. Proceedings of the National Academy of Sciences, 118(30) (2021)).

An aspect of the present invention pertains to the chirality of amino acids. Whereas Glycine is achiral, biosynthetic proteins ordinarily are comprised of L-amino acids, where the chiral center is the alpha-carbon of the amino acid. It is a feature of the present invention that the elements of the glucose-regulated conformational switch—namely, the glucose-binding element attached to one chain and the diol or set of diols attached to the other—may be attached to D-amino acids, such that the positions in space of the switch elements is optimized and, on binding glucose, with least perturbation to receptor-binding affinity. An example of a D-amino-acid substitutions known in the art to perturb receptor binding is provided by the substitution of GlyB8 by D-Ala (Nakagawa, S., et al., Biochemistry 44(13), 4984-99 (2005)). An example of a D-amino-acid substitution known in the art to enhance receptor binding is provided by the substitution of PheB24 by D-Phe (Mirmira, R., and Tager, H. S. J. Biol. Chem. 264(11), 6349-54 (1989)). Pertinent to the placement of switch elements, substitution of GlyA1 by D-amino acids generally preserves receptor-binding affinity whereas substitution of GlyA1 by L-amino acids generally impairs receptor-binding affinity (Wan, Z. L. and Liang, D. C. Scientia Sinica 31(12), 1426-38 (1988); Wan, Z. L. and Liang, D. C. Scientia Sinica 33(7), 810-20 (1990)).

Administration of insulin has long been established as a treatment for diabetes mellitus. A major goal of conventional insulin replacement therapy in patients with diabetes mellitus is tight control of the blood-glucose concentration to prevent its excursion above or below the normal range characteristic of healthy human subjects. Excursions above the normal range are associated with increased long-term risk of microvascular disease, including retinopathy, blindness, and renal failure. Hypoglycemia in patients with diabetes mellitus is a frequent complication of insulin replacement therapy and when severe can lead to significant morbidity (including altered mental status, loss of consciousness, seizures, and death). Indeed, fear of such complications poses a major barrier to efforts by patients (and physicians) to obtain rigorous control of blood-glucose concentrations (i.e., exclusions within or just above the normal range), and in patients with long-established Type 2 diabetes mellitus such efforts (“tight control”) may lead to increased mortality. In addition to the above consequences of severe hypoglycemia (designated neuroglycopenic effects), mild hypoglycemia may activate counter-regulatory mechanisms, including over-activation of the sympathetic nervous system leading to turn to anxiety and tremulousness (symptoms designated adrenergic). Patients with diabetes mellitus may not exhibit such warning signs, however, a condition known as hypoglycemic unawareness. The absence of symptoms of mild hypoglycemia increases the risk of major hypoglycemia and its associated morbidity and mortality.

Multiple and recurrent episodes of hypoglycemia are also associated with chronic cognitive decline, a proposed mechanism underlying the increased prevalence of dementia in patients with long-standing diabetes mellitus. There is therefore an urgent need for new diabetes treatment technologies that would reduce the risk of hypoglycemia while preventing upward excursions in blood-glucose concentration above the normal range.

Diverse technologies have been developed in an effort to mitigate the threat of hypoglycemia in patients treated with insulin. Foundational to all such efforts is education of the patient (and also members of his or her family) regarding the symptoms of hypoglycemia and following the recognition of such symptoms, the urgency of the need to ingest a food or liquid rich in glucose, sucrose, or other rapidly digested form of carbohydrate; an example is provided by orange juice supplemented with sucrose (cane sugar). This baseline approach has been extended by the development of specific diabetes-oriented products, such as squeezable tubes containing an emulsion containing glucose in a form that can be rapidly absorbed through the mucous membranes of the mouth, throat, stomach, and small intestine. Preparations of the counter-regulatory hormone glucagon, provided as a powder, have likewise been developed in a form amenable to rapid dissolution and subcutaneous injection as an emergency treatment of severe hypoglycemia. Insulin pumps have been linked to a continuous glucose monitor such that subcutaneous injection of insulin is halted and an alarm is sounded when hypoglycemic readings of the interstitial glucose concentration are encountered. Such a device-based approach has led to the experimental testing of closed-loop systems in which the pump and monitor are combined with a computer-based algorithm as an “artificial pancreas.”

For more than three decades, there has been interest in the development of glucose-responsive materials for co-administration with an insulin analogue or modified insulin molecule such that the rate of release of the hormone from the subcutaneous depot depends on the interstitial glucose concentration. Such systems in general contain a glucose-responsive polymer, gel or other encapsulation material; and may also require a derivative of insulin containing a modification that enables binding of the hormone to the above material. An increase in the ambient concentration of glucose in the interstitial fluid at the site of subcutaneous injection may displace the bound insulin or insulin derivative either by competitive displacement of the hormone or by physical-chemical changes in the properties of the polymer, gel or other encapsulation material. The goal of such systems is to provide an intrinsic autoregulation feature to the encapsulated or gel-coated subcutaneous depot such that the risk of hypoglycemia is mitigated through delayed release of insulin when the ambient concentration of glucose is within or below the normal range. To date, no such glucose-responsive systems are in clinical use.

A recent technology exploits the structure of a modified insulin molecule, optionally in conjunction with a carrier molecule such that the complex between the modified insulin molecule and the carrier is soluble and may enter into the bloodstream. This concept differs from glucose-responsive depots in which the polymer, gel or other encapsulation material remains in the subcutaneous depot as the free hormone enters into the bloodstream. An embodiment of this approach is known in the art wherein the A chain is modified at or near its N-terminus (utilizing the α-amino group of residue A1 or via the ε-amino group of a Lysine substituted at positions A2, A3, A4 or A5) to contain an “affinity ligand” (defined as a saccharide moiety or diol-containing moiety), the B chain is modified at its or near N-terminus (utilizing the α-amino group of residue B1 or via the ε-amino group of a Lysine substituted at positions B2, B3, B4 or B5) to contain a “monovalent glucose-binding agent.” In this description the large size of the exemplified or envisaged glucose-binding agents (monomeric lectin domains, DNA aptamers, or peptide aptomers) restricted their placement to the N-terminal segment of the B chain as defined above. In the absence of exogenous glucose or other exogenous saccharide, intramolecular interactions between the A1-linked affinity ligand and B1-linked glucose-binding agent was envisaged to “close” the structure of the hormone and thereby impair its activity. Only modest glucose-responsive properties of this class of molecular designs were reported. In this class of analogues the B1-linked agents are typically as large or larger than insulin itself.

The suboptimal properties of insulin analogues modified at or near residue A1 by an affinity ligand and simultaneously modified at or near residue B1 by a large glucose-binding agent (i.e., of size similar or greater than that of an insulin A or B chain) are likely to be intrinsic to this class of molecular designs. Overlooked in the above class of insulin analogues are the potential advantages of an alternative type of glucose-regulated switch engineered exclusively within the B chain without modification of its amino-terminus and without the need for large domains unrelated in structure or composition to insulin. The insulin analogues of the present invention thus conform to one of four design schemes sharing the properties that (a) in the absence of glucose the modified insulin exhibits marked impairment in binding to the insulin receptor whereas (b) in the presence of a high concentration of glucose breakage of a covalent bond to a diol-modified B chain either leads to an active hormone conformation or liberates an active hormone analogue (FIG. 4). Notably the presently disclosed design modifications of the insulin molecule are in each case smaller than the native A or B chains.

Surprisingly, applicant has found that this fundamentally different class of molecular designs may optimally provide a glucose-dependent conformational switch between inactive and active states of the insulin molecule without the above disadvantages. Novel diol modifications of the B chain at or near its C terminus have been previously disclosed in U.S. Provisional Application 63/104,196, entitled “Molecular Designs of Glucose-Responsive and Glucose-Cleavable Insulin Analogues”; incorporated by reference herein. Displacement of the insulin diol from the A-chain-linked glucose-binding element by glucose would lead to detachment of the tethered molecular entity, which in turn enables high-affinity receptor binding; as illustrated in FIGS. 4 and 5, and at the molecular level where 3-fluoro phenylboronic acid (3fPBA) binds reversibly with dihydroxybenzoic acid (DHBA) and other 1,2-diol containing compounds (FIGS. 6A-6D).

The present disclosure relates to polypeptide hormone analogues that contain a glucose-regulated molecular structure or glucose-detachable molecular moiety, designed respectively to either (a) confer glucose-responsive binding to cognate cellular receptors and/or (b) enable glucose-mediated liberation of an active insulin analogue. More particularly, in one embodiment the present disclosure focuses on novel combinations of modified A chains by incorporation of bis-boron-containing glucose-binding elements (GBE) that provide increase opportunity for selective binding to glucose through interaction(s) of two boronic acid moieties binding to one glucose molecule (FIG. 6B), thereby allowing for the design and discovery of novel glucose responsive insulins (GRIs).

In one embodiment the present disclosure is directed to the use of the insulin analogues disclosed herein for use in the treatment of patients and non-human mammals with Type 1 or Type 2 diabetes mellitus by subcutaneous, intraperitoneal or intravenous injection.

The insulin analogues of the present invention may also exhibit other enhanced pharmaceutical properties, such as increased thermodynamic stability, augmented resistance to thermal fibrillation above room temperature, decreased mitogenicity, and/or altered pharmacokinetic and pharmacodynamic properties. More particularly, this invention relates to insulin analogues that may confer either rapid action (relative to wild-type insulin in its regular soluble formulation), intermediate action (comparable to NPH insulin formulations known in the art) or protracted action (comparable to basal insulins known in the art as exemplified by insulin detemir and insulin glargine), such that the affinity of the said analogues for the insulin receptor is higher when dissolved in a solution containing glucose at a concentration above the physiological range (>140 mg/dL; hyperglycemia) than when dissolved in a solution containing glucose at a concentration below the physiological range (<80 mg/dL; hypoglycemia).

### SUMMARY

It is, therefore, an aspect of the present invention to provide insulin analogues that are inactive or exhibit reduced, prolonged activity under hypoglycemic conditions but are activated at high glucose concentrations and so may bind to the insulin receptor with high affinity. The analogues of the present invention contain two essential elements. The first element is a diol-containing side chain in the B chain C-terminal segment (residues B27-B30 or as attached to extended B-chain residues B31 or B32) and/or a C-terminal main chain diol in the B chain; the second is a glucose-binding element (GBE) attached at or near the N terminus of the A chain. This overall scheme is shown in FIGS. 3C, 4, 5 and 6A-6D. Alternatively, in an alternative embodiment (designated “flipped”) the diol-containing side chain is located at or near the N terminus of the A chain and the GBE is attached to an amino acid located at or near the C terminus of the B chain as in FIG. 6D.

One embodiment of the present disclosure pertains to the design and synthesis of glucose-responsive insulins (GRIs) containing modified A chains such that paired boron-containing moieties (BCMs) are tethered at or near the N-terminal residue (position A1); the B chains are modified to comprise diol adducts of arbitrary chemical composition at or near its C terminus. Disclosed herein are specific linkage strategies and chemical approaches for synthesis of A-chain analogues that contain paired BCMs. BCMs may include phenyl-boronic acid (PBA)-based monomeric diol binders (illustrated in FIG. 7) containing carboxylate group (top row), amino group (middle row), and aldehyde group (bottom row) handles to be used as monomeric library inputs. BCMs may also include boronate esters such as benzoxaborole (Bxb), or a combination of PBA and Bxb elements. Such inputs will be reacted with bifunctional scaffolds either built out separately, or prepared by solid-phase peptide synthesis using linker-based chemistries, to produce paired BCMs thus provide bis-boronic acid GBEs (FIGS. 6B-6D, 8). GBEs are specifically positioned at or near the N terminus of the A chain, optionally including modifications of D configuration of amino-acid side chains at position A1 and/or modifications of side chains at one-residue, two-residue or three-residue N-terminal extensions of the A chain (respectively designated positions A₀, A₋₁ and A₋₂).

The A chain of the present invention can thus be the standard 21 residues in length or contain an N-terminal extension of one residue (A₀), two residues (A₋₁-A₀), or three residues (A₋₂-A₋₁-A₀) as illustrated in FIG. 5; or contain (as in insulin-like growth factors; IGFs) a C-terminal extension of 1-5 residues as might enhance the biophysical or biochemical properties of the insulin analogue. The B chain of the present invention can likewise be the standard 30 residues in length or can contain deletion of residues B30, B29-B30, B28-B30 or B27-B30; or can contain an extension to contain additional residue B31 or additional residues B31-B32 as is known in the art. The C-termini of these B chains can carry a carboxylate (as in a conventional peptide) or be modified to contain a main-chain diol.

In accordance with one embodiment, an insulin analogue comprising an insulin A chain and an insulin B chain is provided, wherein the insulin A chain comprises a D-amino acid at position A1 or A₀ and a glucose-binding element covalently linked at or near the insulin A-chain N terminus, optionally attached to the side chain of the amino acid at position A1 or A₀; and the insulin B chain comprises a diol group at or near the C terminus of the insulin B chain, optionally wherein a diol-bearing moiety is linked to the side chain of the amino acid at position B28, B29 or B30 or wherein the diol group is a modified C-terminal amino acid (a) bearing a side chain hydroxyl group and (b) having the C-terminal carboxyl group replaced with CH₂OH, such that the two hydroxyl groups together may bind to an A-chain-linked GBE.

In one embodiment the glucose-binding element is covalently linked to the side chain of an amino acid of the A chain, optionally wherein the glucose-binding element-bearing amino acid is a D-amino acid. In one embodiment the glucose-binding element comprises two or more boron-containing moieties. In one embodiment the glucose-binding element comprises two boron-containing moieties wherein the two boron-containing moieties are linked to one another (extrinsic to the polypeptide), wherein the paired boron-containing moieties are the same or different. In accordance with one embodiment the boron-containing diol-binding element is selected from any of those disclosed in FIG. 7, and in one embodiment the boron-containing moiety is selected from phenylboronic acid (BCM1) elements and benzoboroxole (Bxb) as in BCMs 2-4. While not wishing to be constrained by theory, we envisage that pairwise and higher-order combinations of boron-containing diol-binding moieties, each containing one or more boron atoms, would enhance (a) avidity and the affinity of this combination of boron-containing moieties and the glucose molecule and (b) the cooperativity of glucose-dependent activation of hormonal activity as a function of ambient glucose concentration. Enhanced cooperativity would in turn confer more effective switch-like regulation of glycemia as a function of blood-glucose concentration, a favorable property to mitigate the risk of hypoglycemia in patients with diabetes mellitus. Individual boron-containing diol-binding moieties, as isolated small molecules, are known in the art (for review, see Williams, G. T., Kedge, J. L., and Fossey, J. S. Molecular Boronic Acid-Based Saccharide Sensors. ACS Sens. 6(4), 1508-28 (2021)).

In another embodiment the glucose-binding element bearing insulin A chains of the present disclosure comprises a single amino acid at the N-terminus linked via a peptide bond to the alpha-amino group of the amino acid bearing the glucose-binding element disclosed in FIGS. 5 and 6A. In one embodiment the amino acid positioned at the N-terminus of the glucose-binding-element-bearing A chain is any of the L or D configurations standard 20 amino acids, in another embodiment the N-terminal amino acid is an aliphatic amino acid selected from alanine, glycine, isoleucine, leucine, proline, and valine, and in yet another embodiment the N-terminal amino acid of the glucose-binding element bearing A chain is glycine. In still a further embodiment the N-terminal amino acid of the glucose-binding-element-bearing A chain is in the D-configuration, as depicted in FIGS. 10B, 11A and 12C.

In yet another embodiment the insulin analogue of the present disclosure comprises a B chain that is truncated, lacking residue B30, residues B29-B30, residues B28-B30, residues B27-B30 or residues B26-B30, with a diol group located at the C terminus of the truncated B chain. In another embodiment the native B chain is extended by one or two amino acids with a diol group located at the C terminus of the extended B chain, optionally in conjunction with a diol-modified side chain.

In accordance with one embodiment, an insulin analogue is provided wherein
the insulin A chain is a polypeptide selected from the group consisting of
X₋₁X₀X₁IVEQX₆CX₈SIX₁₁SLYQLENYCN (SEQ ID NO: 4)
X₀X₁IVEQX₆CX₈SIX₁₁SLYQLENYCN (SEQ ID NO: 5), and
X₇₀IVEQX₆CX₈SIX₁₁SLYQLENYCN (SEQ ID NO: 83) wherein
   X₋₁ is any amino acid, optionally wherein X₋₁ is Gly;
   X₀ is any amino acid or a modified amino acid comprising a glucose-binding element linked to its side chain;
   X₁ is any amino acid or a modified amino acid comprising a glucose-binding element linked to its side chain;
   X₆ and X₁₁ are each Cys or selenocysteine; and
   X₈ is Thr, His, Lys, Arg, or Glu;
   X₇₀ is a D-amino acid comprising a glucose-binding element linked to its side chain;
   further wherein at least one of X₋₁, X₀ and X₁ is in the D conformation; and
the insulin B chain is a polypeptide selected from the group consisting of
FVX₂₄QHLCGSHLVEALYLVCGERGFFYTX₄₉X₅₀X₅₁ (SEQ ID NO: 6), and
FVX₂₄QHLCGSHLVEALYLVCGERGFFYTX₅₁ (SEQ ID NO: 7), wherein
   X₂₄ is Lys or Asn;
   X₄₉ is Orn, Glu Asp, Lys, Pro, or a modified amino acid comprising a diol bearing moiety linked to the side chain of the amino acid;
   X₅₀ is Orn, Lys, Pro, or a modified amino acid comprising a diol bearing moiety linked to the side chain of the amino acid; and
   X₅₁ is Thr or a diol bearing amino acid.

In one embodiment the insulin B chain is a polypeptide comprising the sequence of

(SEQ ID NO: 6)
FVX₂₄QHLCGSHLVEALYLVCGERGFFYTX₄₉X₅₀X₅₁,

wherein
X₂₄ is Lys or Asn;
X₄₉ is a modified amino acid comprising a diol bearing moiety linked to the side chain of the amino acid;
X₅₀ is Orn, Lys or Pro; and
X₅₁ is Thr or a diol bearing amino acid. In a further embodiment both X₄₉ and X₅₁ are modified amino acids comprising a diol.

### BRIEF DESCRIPTION OF THE DRAWINGS

FIG. 1A is a schematic representation of the sequence of human proinsulin (SEQ ID NO: 1) including the A- and B-chains and the connecting region shown with flanking dibasic cleavage sites (filled circles) and C-peptide (open circles).

FIG. 1B is a structural model of proinsulin, consisting of an insulin-like moiety and a disordered connecting peptide (dashed line).

FIG. 1C is a schematic representation of the sequence of human insulin including the A-chain (SEQ ID NO: 2) and the B-chain (SEQ ID NO: 3) and indicating the position of residues B27 and B30 in the B-chain.

FIG. 2 is cylinder model of insulin in which the side chains of TyrB16, PheB25 and TyrB26 are shown. The A- and B-chain ribbons are shown in light gray and dark gray, respectively.

FIG. 3A provides a ribbon/cylinder diagram highlighting a potential salt bridge between the C-terminal carboxylate of the B chain (its negative charge is depicted as a minus − within circle at B30) and alpha-amino group of the A chain (its positive charge is depicted as a plus+within circle) as observed in a subset of wild-type insulin crystallographic protomers.

FIG. 3B provides a ribbon/cylinder diagram highlighting a peptide amide bond (box) between the C-terminal carboxylate of the B chain and alpha-amino group of the A chain as observed in inactive single-chain insulin analogues.

FIG. 3C provides a generic scheme in which a diol-modified B chain containing a main-chain hydroxyl group (boxed) in combination with a neighboring hydroxyl group (which may be on a side chain or attached via one or more intervening atoms to the main-chain nitrogen) binds to a glucose-binding element at or near the N terminus of the A chain (horseshoe).

FIG. 4 provides a design scheme of monosaccharide-responsive insulin. The ribbon model of closed inactive insulin (T-state monomer, left) is shown (with a free glucose molecule adjacent to it); the gray box highlights sites of modification (horseshoe shape indicates glucose-binding element; diamond indicates internal diol). The envisioned glucose-regulated conformational cycle in which a monosaccharide acts as a competitive ligand to regulate a conformational switch between the closed state (inactive in absence of ligand) and the open state (active in presence of ligand).

FIG. 5 illustrates sequence of insulin showing A-chain (light; SEQ ID NO: 2) and B-chain (dark; SEQ ID NO: 3) with sites of chemical modification (underlined) including A-chain N-terminal residues A₁, A₀, and GlyA1 and B-chain residues Y B26-T B30 to affect monosaccharide responsivity. Amino acid residues are labelled using their standard single letter codes. Glucose-binding element (horseshoe) is installed at the A-chain N-terminal (positions A₀, A1) as well as at A8. The poly-ol binding group (diamond shape) is installed at the B-chain C-terminal (positions B26-B30) either modified through side chain positions or the main-chain amide nitrogen atom(s).

FIG. 6A depicts the reversible interaction between 3-fluoro phenylboronic acid (3fPBA) placed at or near the A-chain N-terminal with dihydroxybenzoic acid (DHBA) placed in the B-chain Cterminus. Such a reversible interaction between a boronic acid and a diol moiety contributes to the conformation switching between closed ‘inactive’ and open ‘active’ insulin conformations.

FIG. 6B depicts basis for preferential glucose-binding of bis-boronic acids when attached through appropriately placed linker(s) (curved line) (Norrlid, 1995).

FIG. 6C provides a molecular representation of GRI-1 and its conformational switch between the closed state (left) and open state (right).

FIG. 6D illustrates a “flipped” embodiment in which the GBE is attached at or near the C terminus of the B chain whereas the diol moiety or moieties are attached at or near the N terminus of the A chain.

FIG. 7 illustrates phenyl-boronic acid (PBA) based monomeric structures containing carboxylate group (top row, BCM1-4), amino group (middle row, BCM5-8), and aldehyde group (bottom row, BCM9-12) handles to be used as monomeric library inputs. Such inputs will be reacted with bifunctional linkers and linker-based chemistries to produce paired boron-containing monomers (BCMs) to provide bis-boronic acid glucose-binding elements (GBEs).

FIG. 8 Illustrates two distinct and novel strategies for glucose-binding element optimization and frameworks that incorporate either a branchmer-based linker scaffold approach or a Tandem backbone design configuration.

FIG. 9 Illustrates incorporation of aldehyde containing BCMs (9-12) onto the backbone amide nitrogens of A₋₁, A₀, A1 positions in the insulin A-chain as an alternate approach to tandem backbone modification that directly modifies the backbone amide bonds of these preferred positions.

FIG. 10A depicts potential glucose sensors prepared on solid phase resins (see FIG. 12A) containing symmetrically paired boron-containing monomer (BCM2) attached through linkers having four (4), six (6), and six (6) atom spacer respectively. Each paired BCMs has a thiol-based handle for chemoselective conjugation reaction onto specifically designed and positioned SH reactive group on an insulin framework as in FIGS. 12B, 13, 14 and applied to the synthesis of GRIs 1-4. Each paired BCM containing structures design for the inherent selectivity of boronic acids toward monosaccharides and how two phenyl-boronic acids can provide a path for glucose selectivity thru spatial disposition and molecular complementarity. The general structure of this arrangement employs symmetrical benzoboroxole (BCM2) units bound through a diamino propionyl-cysteinyl linker with or without intervening amino acids denoted as (GBE1-C, GBE2-C, and GBE3-C) to enhance glucose-binding.

FIG. 10B provides a general scheme for the combinatorial design of paired BCMs illustrating three (3) different branch-mer configurations carrying a C-terminal Cys residue which provides a “thiol handle” (via nitropyridyl-sulfenyl-based activation, FIG. 12B) to produce a disulfide linkage to the single-chain amino group of DL-Cys (A₀) (upper row) or D-Cys (A₁) (lower row) at or near the A-Chain N-terminus. The left upper and lower panels illustrate use of a Dap (n=1) or Dab (N=2) linker scaffold whose amino groups can be selectively and unambiguously modified with carboxylate containing BCMs 1-4 to prepare either symmetrical or asymmetrical paired BCMs. The middle and right two panels illustrate a similar design strategy but also include an N-terminal amino acid insertion (center right) or side-chain amino acid insertion (far right), where R represents single-chain of any proteogenic L-amino acid or D-amino acid. Such changes are useful for optimizing the GBE to specific polyol groups of interest (i.e., glucose or fructose selectivities). The right two panels illustrate modifications of the A-Chain N-terminal with BCMs 1-4 while the single-chain amino group at A₀ or A₁ is extended on the B-chain N-terminal by a single residue comprising any proteogenic L-amino acid or D-amino acid. In all cases BCMs 1 thru 5 can be selectively and unambiguously coupled to any of position indicated.

FIG. 11A provides a general scheme for the combinatorial design of paired BCMs illustrating three (3) different branch-mer configurations such that GBEs are linked through an amide bond to the side-chain amino group of a basic amino acid (e.g., Dap, Dab, Orn, or Lys; left panel, n=1-4 respectively). The left panel illustrates either the N-terminal amino-acid insertion (far left) or side-chain amino acid insertion (second left), where R represents single-chain of any proteogenic L-amino acid or D-amino acid or non-natural amino acids. Such changes are useful for optimizing the GBE to specific polyol groups of interest (i.e., glucose or fructose selectivities). The center two panels illustrate modifications of the A-chain N-terminal with BCMs 1-4 while the side-chain amino group at A₀ or A1 is extended by one residue via any proteogenic L-amino acid or D-amino acid. The far right structure illustrate elaboration of both A₀ and A₁ positions with basic amino acids to install GBE1-4 in both positions where A₋₁ is any L-amino acid. In all cases BCMs 1-4 can be selectively and unambiguously coupled to any of these five amino position.

FIG. 11B provides explicit structures of the glucose-binding elements (GBEs) that are defined as GBE1, GBE2, GBE3, GBE4, GBE5, and GBE6 containing paired BCM2 groups attached to the branch-mers (Dap and Dab). In GBEs 1 and 4 the BCMs are directly linked to both amino groups of the Dap or Dab amino groups to as exemplified in the left panel. GBEs 2 and 5 (center panel) differ from GBE land 4 in that the alpha amino group of Dap (n=1) or Dab (n=2) is modified with the amino acid glycine prior to paired BCM2 coupling, denoted as BCM2-Xxx-Dap(BCM2)-OH (GBE2) and BCM2-Dab(BCM2-Xxx)-OH (GBE5) respectively, where Xxx=Gly. The GBEs 3 and 6 (left panel) differ from GBE land 4 in that the sidechain amino group of Dap (n=1) or Dab (n=2) is modified with the amino acid, denoted as BCM2-Xxx-Dab(BCM2)-OH (GBE3) and BCM2-Dab(BCM2-Xxx)-OH (GBE6) respectively, where Xxx=glycine, prior to paired BCM2 coupling. The use of Xxx recognizes the substitution by any other L-AAs, D-AAs, or non-natural AAs would create unique GBEs thus allowing for modulation of binding properties of each GBE with respect to affinity and selectivities toward glucose, other monosaccharides, or polysaccharide.

FIG. 11C provides six specific examples of symmetrical placement of paired BCM2 on the amino groups of either Dap (n=1) or Dab (n=2) to produce sidechain amide linked GBE1 thru GBE6 onto the insulin A chain at positions A₀ (replaced by Dap) or A1 (replaced by D-Dap) the insulin sequence

FIG. 12A depicts reaction schemes used for solid phase synthesis of Glucose-binding element (GBE) GBEs 1-6 herein explicitly built out to connect (1) GBEs 4, 5, 6, onto the side chain amino group of Dab A₀, and D-Dab at the A₀ and A₁ positions on insulin sequences (also see FIGS. 15 and 16), (2) on the octapeptide sequence, GFFYTX(GBE1-6)PT (SEQ ID NO: 41) that represents the octapeptide (8P) sequence of insulin lispro (KP) residues B23-B30, where X=Lys, Orn, Dab, and Dap, and (3) any ‘acid’ (ie. Wang, Cl-Trtyl) or ‘amide’ (ie. Rink, RAM, aminomethyl) functionalized solid supports. A similar approach using Dap or D-Dap was used to install GBEs 1, 2, and 3 onto the A₀ and A1 positions of the insulin analogues.

FIG. 12B depicts resultant molecule GBE1-Cys(SH)—OH prepared on H-Cys(Trt)-O-Wang or H-Cys(Trt)-O—Cl-Trt- resins employing the reaction scheme shown in FIG. 12A but using Fmoc-Dap(Fmoc)-OH in place of Fmoc-Dab(Fmoc)-OH in far left column. The free SH group of GBE1-Cys(SH)—OH is activated using DBNP (2,2-Dithiobis(5-nitropyridine)) in TFA to produce GBE1-C(Npys)-OH which then allows for conjugation to Cys(SH)A₀ or dCys(SH)A1 synthetic insulin precursors to produce the disulfide linked GBE-insulin intermediates.

FIG. 13 depicts the synthetic reaction scheme to prepare GA₋₁, Cys(SH)A₀ or GA₀, dCys(SH)A1 modified DesDi-insulin and the coupling of GBE1-C(Npys)-OH to form the disulfide linked GBE intermediates onto their respective frameworks. Specific examples include four single-chain insulins (B1-B28-A1A21) frameworks carrying native disulfide pairing and the Selenocysteine (SecA6-SecA11) bridge substitutions as precursors to GRIs 1-4.

FIG. 14 depicts GlyA-1, Cys(GBE-1)A₀, SecA6, SecA11, Lys(DHBA)B29-lispro and GA₀, dC(GBE-1)A1, SecA6-SecA11, Lys(DHBA)B29-lispro insulin synthetic scheme to prepare disulfide linked (GBE1C) GBEs onto the L-CysA₀ or D-CysA1 modified DesDi-insulin frameworks (single-chain insulins (B1-B28-A1A21) containing selenocysteine (SecA6-SecA11) diselenide-bridge substitution. Desoctapeptide (DOI) precursors were used in Trypsin mediated coupling reaction using Lys(DHBA)B28 modified octapeptides (SEQ ID NO: 10), an N-terminally modified A chain comprising a GBE-1 at position A0 (SEQ ID NO: 8) linked via disulfide bonds to a C-terminally truncated B chain (SEQ ID NO: 9). A similar scheme was used for the native disulfide bridge containing analogues and for frameworks modified with GBE2-C and GBE3-C disulfide bond connections.

FIG. 15 depicts the generalized solid phase synthetic scheme to prepare and install amide bond linked glucose-binding elements (GBEs 1-6). Illustrated here for GlyA₀, [D-Dap(GBE4)]A1, and GlyA₀, [D-Dap(GBE5)]A1, DesDI single-chain insulin intermediates. The same scheme was also used to prepare additional DesDi single-chain analogues carrying GBEs 1-6.

FIG. 16 depicts the generalized solid phase synthetic scheme to prepare and install amide bond linked glucose-binding elements (GBEs 1-6) as a GlyA-1, [Dap(GBE1)]A1, HisA8 and GA₋₁, [Dap(GBE3)]A1, HisA8 DesDi single-chain insulin intermediates (SEQ ID NO: 11). The same scheme was also used to prepare additional DesDi single-chain analogues carrying GBEs 1-6.

FIG. 17 provides control studies of insulin lispro (“KP” in inset legends) demonstrating absence of glucose dependence (±50 mM glucose; upper) and absence of fructose dependence (±50 mM fructose; lower). In each panel extent of pIR/IR phosphorylation is shown (vertical axis; normalized to initial pIR/IR ratio in absence of hormone) as a function of insulin analogue concentration in nanomolar units (horizontal axis). Data obtained in the absence (or presence) of a monosaccharide are shown as blue diamonds (or orange squares). Abbreviations: IR and pIR, insulin receptor and phosphorylated insulin receptor, respectively.

FIG. 18 demonstrates monosaccharide-dependent biological activity of a derivative of insulin lispro (GRI-2, in inset legends) where GRI-2 is defined as analogue GlyA₋₁, Cys(GBE1-C)A₀, Lys(DHBA)B28-lispro. The A chain contains a two-residue N-terminal extension (Gly at position A-1 and Cysteine at position A₀), and modification of the CysA₀ side-chain thiol group by a paired BCM2 in the form of GBE1-C (see FIG. 14). The B chain is modified at the epsilon-amino group of LysB28 by 2,3-dihydroxybenzoic acid (DHBA). The two-panel format is as in FIG. 17, respectively testing glucose dependence (±50 mM glucose; upper) and absence of fructose dependence (±50 mM fructose; lower). In each panel extent of pIR/IR phosphorylation is shown (vertical axis; normalized to initial pIR/IR ratio in absence of hormone) as a function of insulin analogue concentration in nanomolar units (horizontal axis). Data obtained in the absence (or presence) of a monosaccharide are shown as blue diamonds (or orange squares). Abbreviations: IR and pIR, insulin receptor and phosphorylated insulin receptor, respectively.

FIG. 19 demonstrates monosaccharide-dependent biological activity of a derivative of insulin lispro (GRI-3, in inset legends) where GRI-3 is defined as analogue GlyA₋₁, Cys(GBE1-C)A₀, SecA6, SecA11, Lys(DHBA)B28-lispro. The A chain contains a two-residue N-terminal extension (Gly at position A₋₁ and Cysteine at position A₀), and modification of the CysA₀ side-chain thiol group by a paired BCM2 2 in the form of GBE1-C (see FIG. 14. The B chain is modified at the epsilon-amino group of LysB28 by 2,3-dihydroxybenzoic acid (DHBA). The two-panel format is as in FIG. 17, respectively testing glucose dependence (±50 mM glucose; upper) and absence of fructose dependence (±50 mM fructose; lower). In each panel extent of pIR/IR phosphorylation is shown (vertical axis; normalized to initial pIR/IR ratio in absence of hormone) as a function of insulin analogue concentration in nanomolar units (horizontal axis). Data obtained in the absence (or presence) of a monosaccharide are shown as blue diamonds (or orange squares). Abbreviations: IR and pIR, insulin receptor and phosphorylated insulin receptor, respectively.

FIG. 20 demonstrates monosaccharide-dependent biological activity of a derivative of insulin lispro (GRI-4 in inset legends) where GRI-4 is defined as insulin analogue GlyA₀, D-Cys(GBE1-C)A1, SecA6, SecA11, Lys(DHBA)B28Pro-Iispro. The A chain contains a one-residue N-terminal extension (Gly at position A₀), substitution of Glycine at A1 by D-Cysteine, and modification of the D-CysA1 side-chain thiol group by a paired BCM2 in the form of GBE1-C (see FIG. 14). The B chain is modified at the epsilon-amino group of LysB28 by 2,3-dihydroxybenzoic acid (DHBA). The two-panel format is as in FIG. 17, respectively testing glucose dependence (±50 mM glucose; upper) and absence of fructose dependence (±50 mM fructose; lower). In each panel extent of pIR/IR phosphorylation is shown (vertical axis; normalized to initial pIR/IR ratio in absence of hormone) as a function of insulin analogue concentration in nanomolar units (horizontal axis). Data obtained in the absence (or presence) of a monosaccharide are shown as diamonds (or squares). Abbreviations: IR and pIR, insulin receptor and phosphorylated insulin receptor, respectively.

FIGS. 21A-21C depicts cell signaling assay for GRI-3, defined as insulin analogue GlyA₀, D-Cys(GBE1-C)A1, SecA6, SecA11, Lys(DHBA)B28-lispro and glucose-dependent IR phosphorylation (FIG. 21A) and glucose-dependent gene regulation in HepG2 cells in culture with respect to a representative glucogenogenic gene (PEPCK; FIG. 21B) and genes regulating lipid biosynthesis (ChREBP and SREBP; FIG. 21C). Data in (FIG. 21A) are remarkable for Time in range (TIR) and interconversion of glucose units (mg/dL and mM) are shown in schematic form at right.

FIG. 22 illustrates potency comparison following intravenous injection of insulin lispro versus GRI-1 (composition GlyA₀, D-Cys(GBE1-C)A1, Lys(DHBA)B28-lispro) in streptozotocin-induced (STZ)-diabetic rats. Panel A. Diabetic rats were injected intravenously at t=0 with 9 nmol/kg of insulin lispro (n=7), GRI-1 (n=7), or diluent (n=29) and changes in blood-glucose concentrations were monitored. Insulin lispro is shown as filled red square, GRI-1 as filled blue circles, and diluent as X. Panel B. Area over the curve (AOC) is calculated. Mean±SEM; *denotes P<0.05. These data show that GlyA₀, D-Cys(GBE1-C)A1, Lys(DHBA)B28-lispro potency is approximately one half that of insulin lispro.

FIG. 23 depicts subcutaneous injections of insulin lispro, GRI-1=GlyA₀, D-Cys(GBE1-C)A1, Lys(DHBA)B28-lispro (GRI-1), and diluent in STZ-diabetic rats. Diabetic rats were injected subcutaneously at t=0 with 6 nmol/kg of lispro, red squares (n=16), 12 nmol/kg of insulin lispro, green triangles (n=16), GRI-1 using 6 (blue circles, n=12), 12 (purple diamonds, n=12), and 24 (black X, n=11) nmol/kg, respectively, or diluent (orange X, n=17) and changes in blood-glucose concentrations were monitored. Panel B. Area over the curve (AOC) is calculated. Mean±SEM; *denotes p<0.05.

FIGS. 24A-24C illustrate the glucose clamp protocol and graphical results using insulin lispro, GRI-1, and diluent. FIG. 24A illustrates the time of administration and measurement schemes across the glucose clamp protocol. Rats were food deprived for 12 h. Somatostatin (40 ug) was injected subcutaneously at t=−20 min and again intravenously at t=0 min, and somatostatin was continued as an intravenous infusion at 2 ug/min thereafter. Rats were injected subcutaneously (at 5 min) with insulin lispro at 2.8 nmol/kg, GRI-1=GlyA₀, D-Cys(GBE1-C)A1, Lys(DHBA)B28-lispro at 7.0 nmol/kg, or diluent. Glucose infusion rate was adjusted to reach the desired glycemic level by t=15 min; glucose infusion was continued up to 90 min to maintain the desired glycemic level. FIG. 24B provides an example of the glucose infusion rate (GIR) protocol using 2.8 nmol/kg of insulin lispro or diluent aimed at a blood-glucose target concentration of 250 mg/dL. The difference between the glucose infusion rate (GIR) for insulin lispro (purple wavy line) and diluent (blue line) represents the GIR attributable to the effect of insulin. FIG. 24C depicts results of GIR attributable to insulin lispro (left) and GRI-1 (right) are shown. Data from 60 separate experiments using insulin lispro, GRI-1, and diluent clamped at different glucose levels are summarized in the bar graphs and FIG. 25. The GIR values using insulin lispro at different glucose levels is not significantly different from each other, while GIR values using GRI-1 are significantly higher at elevated glucose levels. Mean±SEM; NS denotes not significant.

FIG. 25 lists the data calculation from FIG. 24C in the euglycemic and hyperglycemic clamp protocol. A. In the absence of any insulin injection (diluent-injected), the Glucose Infusion Rate (GIR) necessary to maintain blood-glucose concentrations at several pre-selected targets is shown (mg glucose/kg/h). B. The GIR necessary to maintain blood-glucose concentrations at several pre-selected target levels following subcutaneous injection of 5.0 μg insulin lispro (2.8 nmol/kg) is shown. The GIR attributable to the effect of insulin lispro is calculated as the difference in the GIR using insulin lispro and diluent. C. GIR necessary to maintain blood-glucose concentrations at several pre-selected target levels following subcutaneous injection of 12.5 μg of GRI-1 (7.0 nmol/kg) is shown; the GIR attributable to the effect of GRI-1 is calculated as the difference in the GIR between use of GRI-1 insulin and diluent (from panel A). Data are shown as mean±SEM.

FIGS. 26A and 26B illustrate the bi-phasic glucose clamp protocol and graphical results using GRI-1=GlyA₀, D-Cys(GBE1-C)A1, Lys(DHBA)B28-lispro and diluent. FIG. 26A. illustrates the time of administration and measurement schemes across the glucose clamp protocol. Rats were food deprived for 12 h. Somatostatin (40 ug) was injected subcutaneously at t=−20 min and intravenously at t=0 min, and continued as an intravenous infusion at 2 ug/min thereafter. At 5 min, rats were injected subcutaneously with GRI-1 (7.0 nmol/kg, or diluent). During Phase A (5-30 min), glucose infusion rate was adjusted to a target blood-glucose concentration of 85 mg/dL. Phase B (40-70 min): at 30 min, glucose infusion was increased to reach the goal of 200 mg/dL by 40 min; glucose infusion was then continued to maintain the blood-glucose concentration of 200 mg/dL. FIG. 26B: Results of GIR attributable to GRI-1 at blood-glucose concentrations of 85 and 200 mg/dL are shown. Data from 16 separate experiments using diluent or GRI-1 are summarized in the bar graphs (FIG. 27). Mean±SEM; *denotes p<0.05.

FIG. 27 lists the data calculations from FIG. 26B in the bi-phasic glucose clamp protocol and results using GRI-1 and diluent. Panel A. Rats were food deprived for 12 h. Somatostatin (40 ug) was injected subcutaneously at t=−20 min and intravenously at t=0 min, and somatostatin was continued as an intravenous infusion at 2 ug/min thereafter. At 5 min, rats were injected subcutaneously with GRI-1 (7.0 nmol/kg) or diluent. During Phase A (5-30 min), glucose infusion rate was adjusted to maintain a target blood-glucose concentration of 85 mg/dL. Then at 30 min, glucose infusion was increased to reach the goal of 200 mg/dL by 40 min; glucose infusion was then continued to maintain the blood-glucose concentration of 200 mg/dL during Phase B (40-70 min). Panel B. Results of GIR attributable to GRI-1 at blood-glucose concentrations of 85 and 200 mg/dL are shown. Data from 16 separate experiments using diluent or GRI-1 are summarized. The GIR attributable to GRI-1 is significantly higher at a blood-glucose concentration of 200 compared to 85 mg/dL. Plots provide mean±SEM.

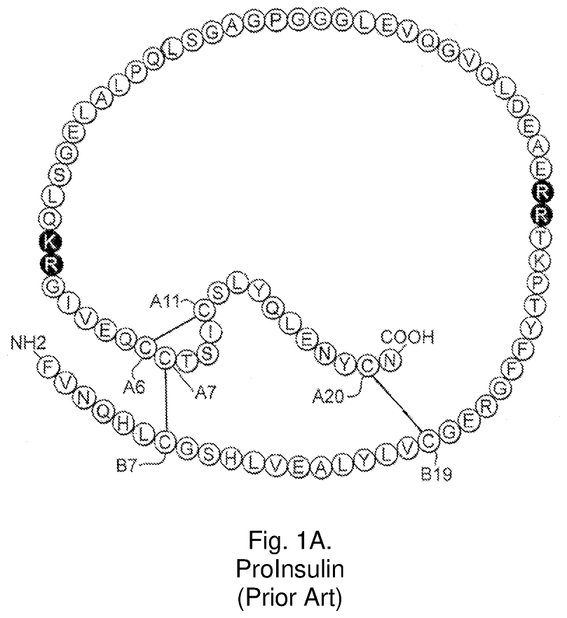

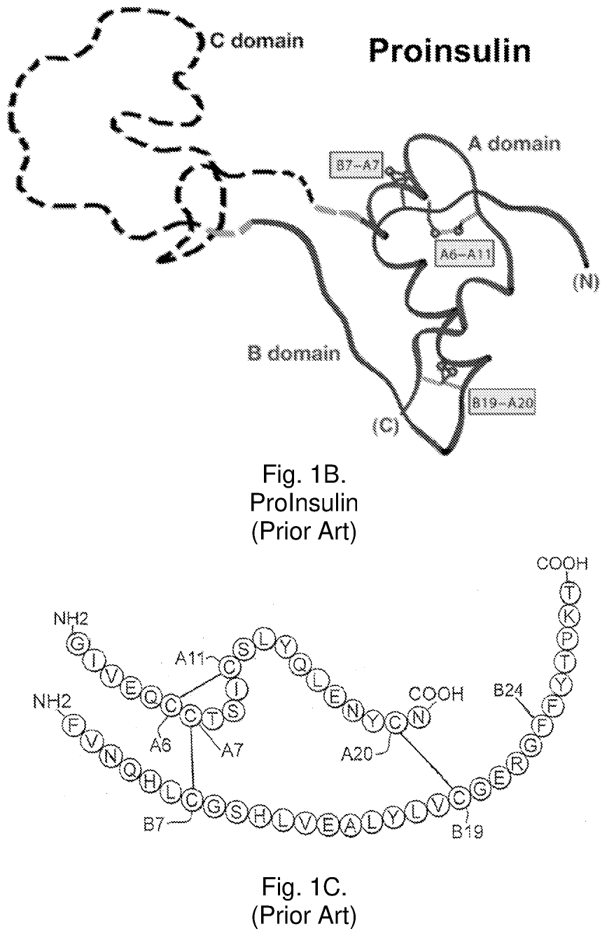

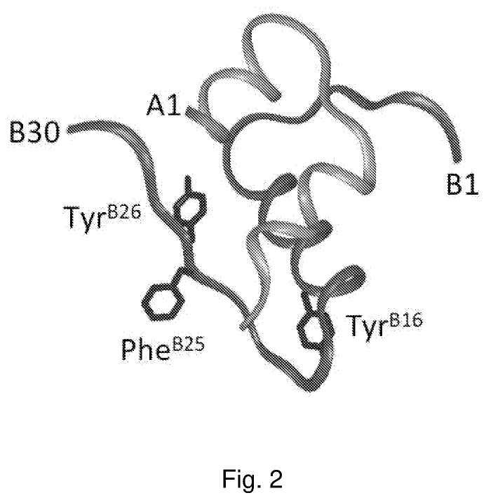

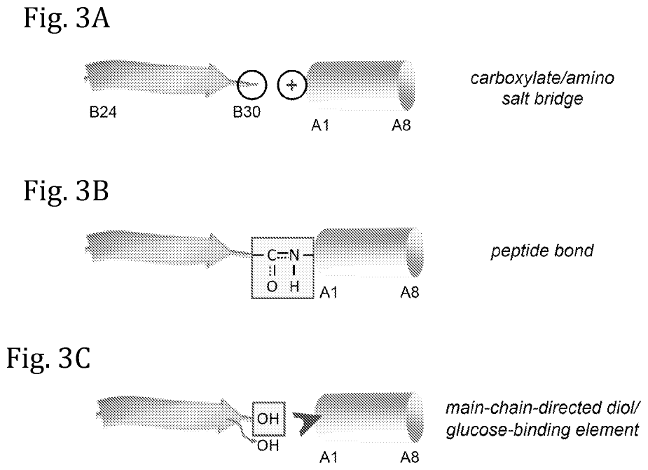

### DETAILED DESCRIPTION

### Abbreviations

BCMs Boron-containing moieties
Bxb Benizoxaborole
CM Chem Matrix
6-Cl-HOBt 6-chloro-1-hydroxybenzotriazole
DHBA Dihydroxybenzoic acid
DIC N,N′-Diisopropylcarbodiimide
DOI des-octapeptide insulin
FRI Fructose-responsive insulin
GBE Glucose binding elements
GRI Glucose-responsive insulin
HMBA 4-{3-methoxy-4-[(L-phenylalanyloxy)methyl]phenoxy}butyric acid
PBA Phenylboronic acid
TFA trifluoroacetic acid
SCI single chain insulin
STZ Streptozotocin
TIS triisopropylsilane
KP Lispro insulin

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein the term “patient” without further designation is intended to encompass any warm blooded vertebrate domesticated animal (including for example, but not limited to livestock, horses, cats, dogs and other pets) and humans receiving a therapeutic treatment with or without physician oversight.

The term “inhibit” defines a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, the term “Diol-bearing amino acids” comprise natural or unnatural amino acids whose side chains are modified by linkage of a diol moiety to an attachment point located on the amino acid side chain. Examples of side chain attachment points are provided by a thiol function, amino function or carboxylate function. Amino-containing side chains, for example, may be provided by the natural amino acid Lysine or by unnatural amino acids Ornithine (Orn), Diaminobutyric Acid (Dab) and Diaminoproprionic acid (Dap), each in either the L- or D configuration. Thiol-containing amino acids, as a further example, may be the natural amino acid Cysteine or the unnatural amino acid Homocysteine, each either in the L- or D configuration. Diol-bearing amino acids represent the covalent combination of (a) the above recited amino acids and (b) a diol moiety exemplified (but not restricted to) those listed in Table 2.

As used herein, the term “threoninol” absent any further elaboration encompasses L-allo-threoninol, D-threoninol and D-allo-threoninol.

As used herein the term “main-chain” defines the backbone portion of a polypeptide, and distinguishes the atoms comprising the backbone from those that comprise the amino acid side chains that project from the main-chain.

As used herein, the term “pharmaceutically acceptable salt” refers to those salts with counter ions which may be used in pharmaceuticals. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. Such salts include:
(1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D)- or (L)-malic acid, maleic acid, methane sulfonic acid, ethanesulfonic acid, p-toluenesulfonic acid, salicylic acid, tartaric acid, citric acid, succinic acid or malonic acid and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

Acceptable salts are well known to those skilled in the art, and any such acceptable salt may be contemplated in connection with the embodiments described herein. Examples of acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable acceptable salts are found in Remington's Pharmaceutical Sciences, 17th Edition, Mack Publishing Company, Easton, Pa., 1985.

As used herein the term “native insulin peptide” is intended to designate the 51 amino acid heterodimer comprising the A chain of SEQ ID NO: 2 and the B chain of SEQ ID NO: 3, as well as single-chain insulin analogues that comprise SEQ ID NOS: 2 and 3. The term “insulin peptide” as used herein, absent further descriptive language is intended to encompass the 51 amino acid heterodimer comprising the A chain of SEQ ID NO: 2 and the B chain of SEQ ID NO: 3, as well as heterodimers and that comprise modified derivatives of the native A chain and/or B chain. An “insulin A chain” is defined as the 21 amino acid sequence of SEQ ID NO: 2 as well as any modified derivatives of the native A chain, and an insulin B chain is defined as the 30 amino acid sequence of SEQ ID NO: 3 as well as any modified derivatives of the native B chain. Modified derivatives of the “insulin peptide”, “insulin A chain” and “insulin B chain” included one or more amino-acid substitutions at positions selected from A1, A5, A8, A9, A10, A12, A14, A15, A17, A18, A21, B1, B2, B3, B4, B5, B9, B10, B13, B14, B17, B20, B21, B22, B23, B26, B27, B28, B29 and B30, or deletions of any or all of positions B1-4 and B26-30, or the addition of 1-3 amino acids to the N-terminus of the A chain or at the C-terminus of the B chain. Additional amino acids linked to the insulin A chain peptide at the N-terminus are numbered starting with 0 and increasing in negative integer value as they are further removed from the native insulin A chain sequence. For example, the position of an amino acid within an N-terminal extension of the A chain is designated A₋₁ or A₀, wherein A₀ represents the position of an amino acid added directly through the use of a standard amide-bond connectivity to the native N-terminal amino acid of the insulin A chain, and A₋₁ represents the position of an amino acid having a single amino acid intervening between the A₋₁ amino acid and the native N-terminal A1 amino acid of the insulin A chain.

As used herein, an amino acid “modification” refers to a substitution, addition or deletion of an amino acid through amide bond coupling or other amide bond isosteric mimetice bond connectivity, or the derivation of an amino acid by the addition and/or removal of chemical groups to/from the amino acid, and includes substitution with or addition of any of the 20 amino acids commonly found in human proteins, as well as atypical or non-naturally occurring amino acids. Commercial sources of atypical amino acids include Sigma-Aldrich (Milwaukee, WI), ChemPep Inc. (Miami, FL), and Genzyme Pharmaceuticals (Cambridge, MA). Atypical amino acids may be purchased from commercial suppliers, synthesized de novo, or chemically modified or derivatized from naturally occurring amino acids.

As used herein an amino acid “substitution” refers to the replacement of one amino acid residue by a different amino acid residue. Throughout the application, all references to a particular amino acid position by letter and number (e.g. position A5) refer to the amino acid at that position of either the A chain (e.g. position A5) or the B chain (e.g. position B5) in the respective native human insulin A chain (SEQ ID NO: 2) or B chain (SEQ ID NO: 3), or the corresponding amino acid position in any analogues thereof. For example, a reference herein to “position B28” absent any further elaboration would mean the corresponding position B27 of the B chain of an insulin analogue in which the first amino acid of SEQ ID NO: 3 has been deleted.

As used herein, the term “conservative amino acid substitution” is defined herein as exchanges within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys; Ornithine (Orn)
IV. Large, aliphatic, nonpolar residues:
   Met, Leu, Ile, Val, Cys, Norleucine (Nle), homocysteine
V. Large, aromatic residues:
   Phe, Tyr, Trp, acetyl phenylalanine

As used herein a “linker” is a bond, molecule or group of molecules that binds two separate entities to one another. In one embodiment the linker provides optimal spacing of the two entities.

As used herein a “glucose-binding elements (GBE)” is molecular structure that comprises two or more boron-containing moieties (BCMs). A bis-boronic acid GBE is a GBE comprising two boron-containing moieties wherein the two boron-containing moieties are linked to one another either directly or through a linker.

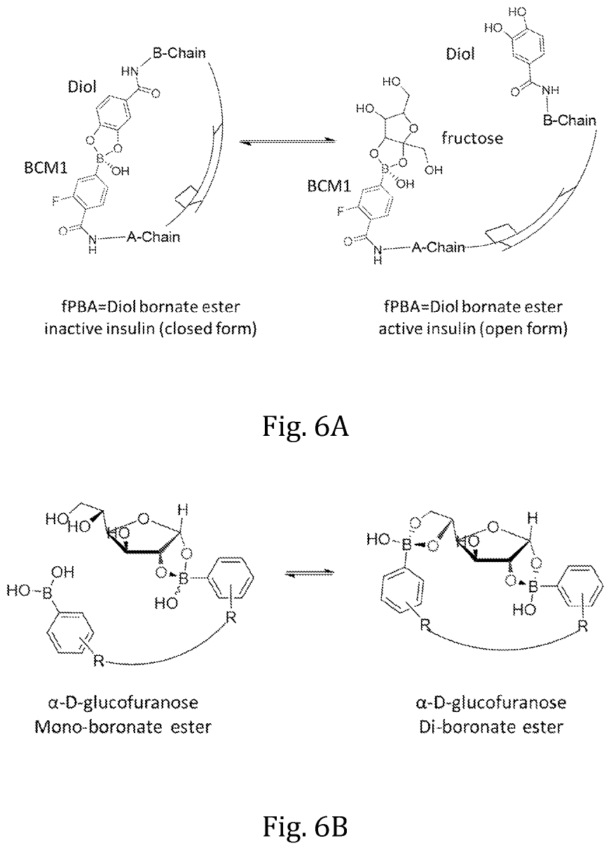

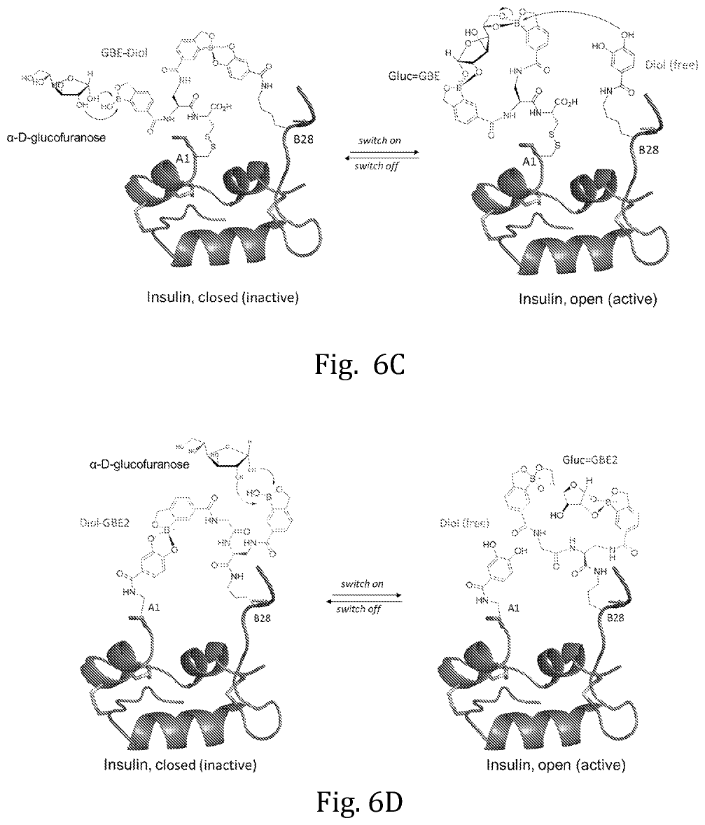

As used herein a “boron-containing moiety (BCM)” is a chemical structure comprising a boron molecule covalently linked to two oxygen atoms, wherein the boron-containing moiety is capable of interacting with a diol containing entity to form a reversible covalent link between the diol and the boron atom (see FIGS. 6A-6D).

As used herein a “a glucose responsive insulin (GRI)” is an insulin analogue that comprises a glucose-binding elements (GBEs). Such GRIs are inactive or exhibit reduced activity relative to native insulin under hypoglycemic conditions but become activated at elevated glucose concentrations, optionally at blood-glucose concentrations greater than 150 mg/dL after 8 hours of fasting, and bind the insulin receptor with high affinity.

### Representative Embodiments

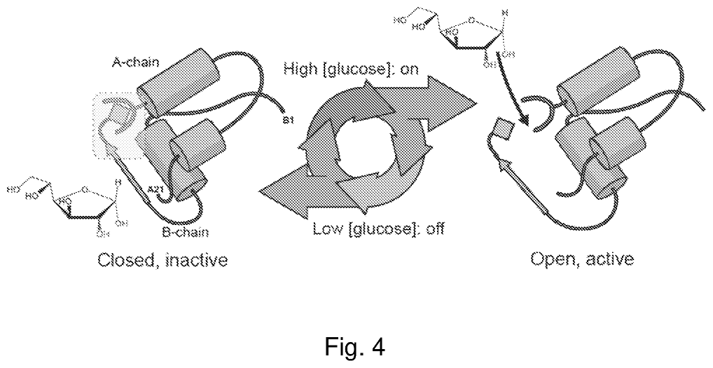

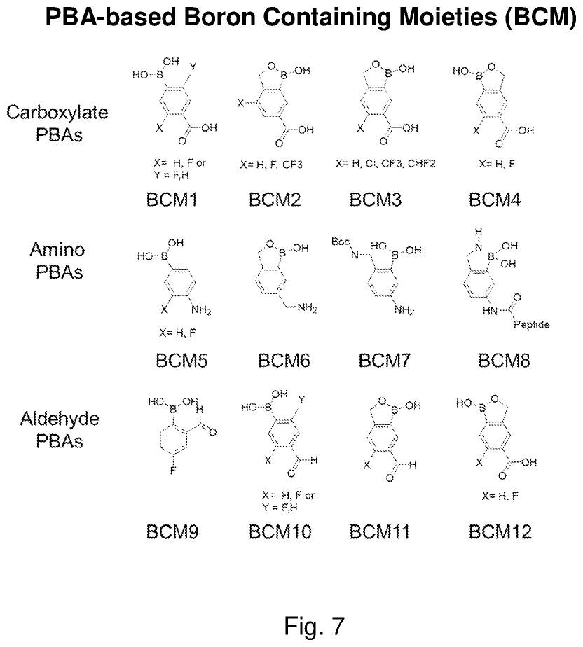

As disclosed herein analogues of native insulin have been prepared that are biological sensors that activate only under hyperglycemic conditions. In one embodiment the insulin analogue comprises an insulin A chain peptide modified by the linkage of a glucose-binding element at or near its N terminus and a B chain modified by the linkage of one or more diol adducts at or near its C terminus, wherein the glucose-binding element comprises two or more boron-containing diol-binding elements disclosed in FIGS. 4, 5, 6A, 6C. In another embodiment the insulin analogue comprises an insulin B chain peptide modified by the linkage of a glucose-binding element at or near its C terminus comprising two or more boron-containing diol-binding elements and containing an A chain modified by linkage of (i) one or more diol adducts at or near its N terminus and/or (ii) one or more glucose-binding elements (together or singly containing two or more boron atoms) at or near its N terminus disclosed in FIG. 6D. In accordance with one embodiment the boron-containing diol-binding element is selected from any of those disclosed in FIG. 7. In one embodiment the boron-containing diol-binding element of the glucose-binding element is selected from phenylboronic acid and benzoboroxole. In one embodiment the N-terminus of the insulin A chain peptide is modified to comprise two glucose-binding elements wherein the boron containing moieties comprising the glucose-binding elements are the same or different.

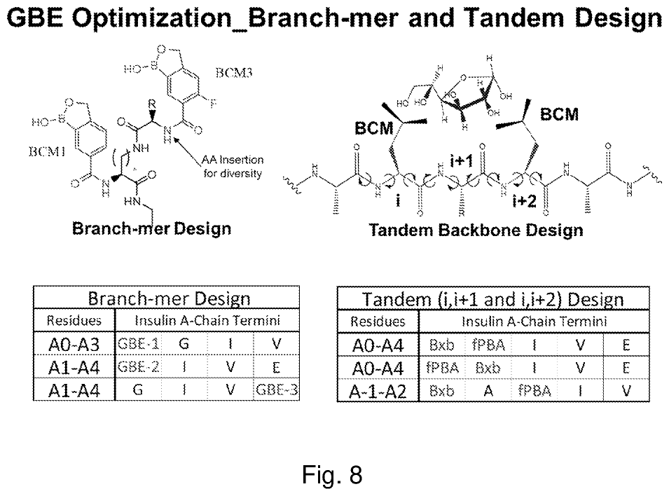

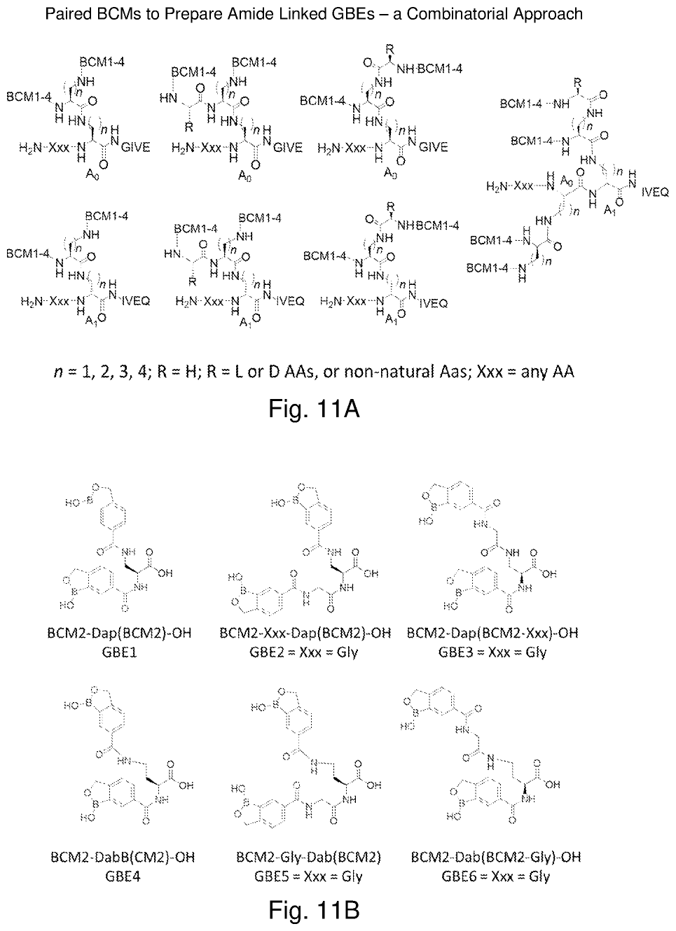

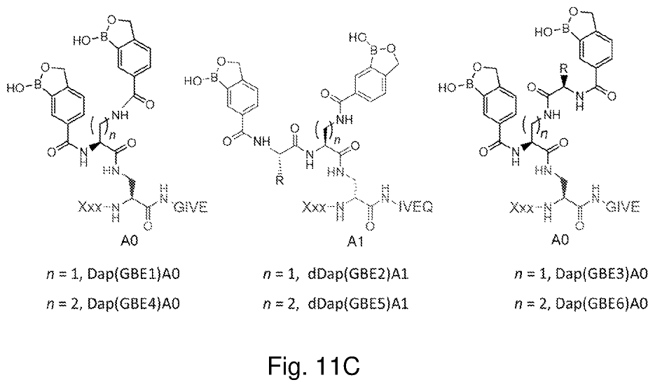

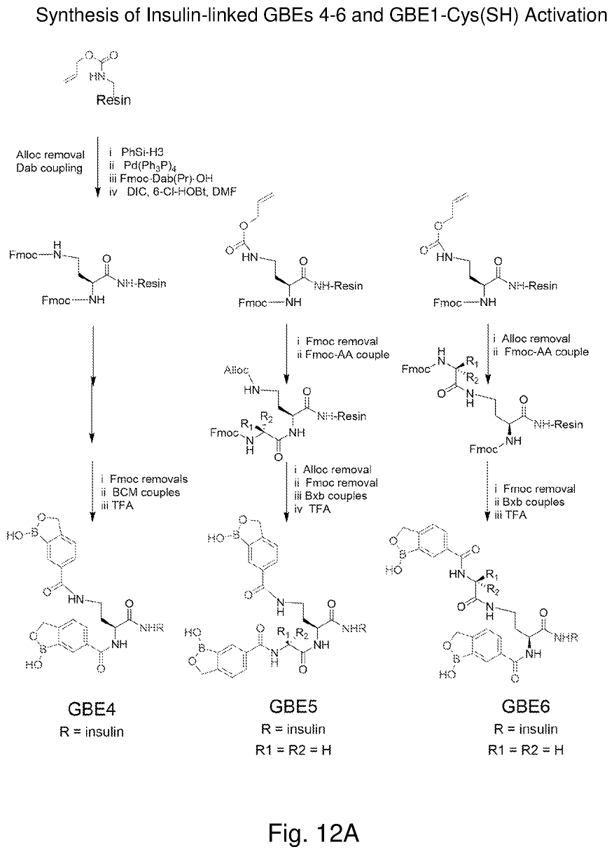

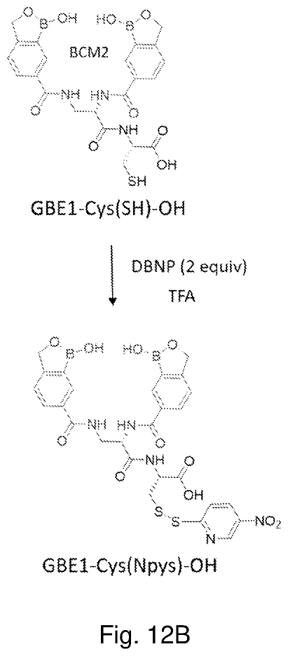

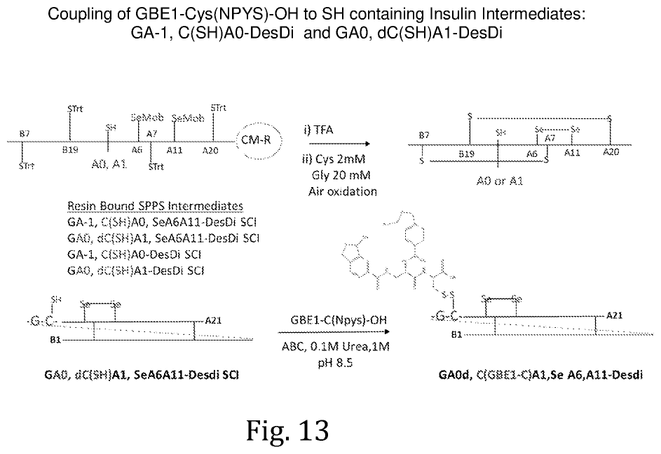

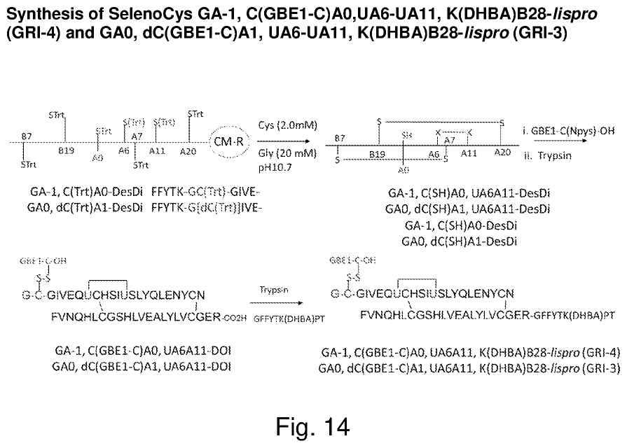

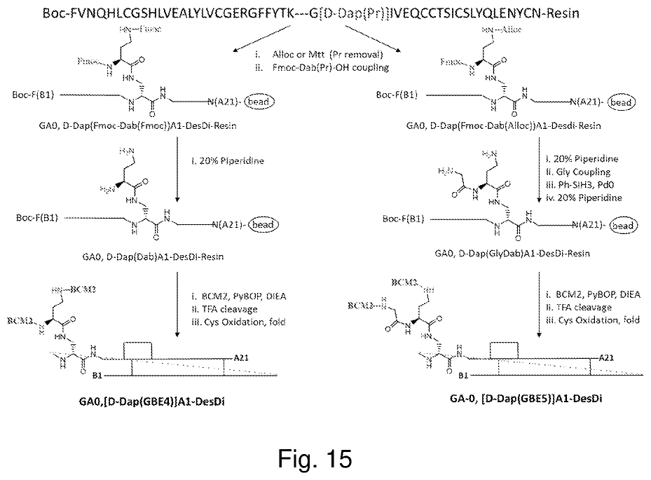

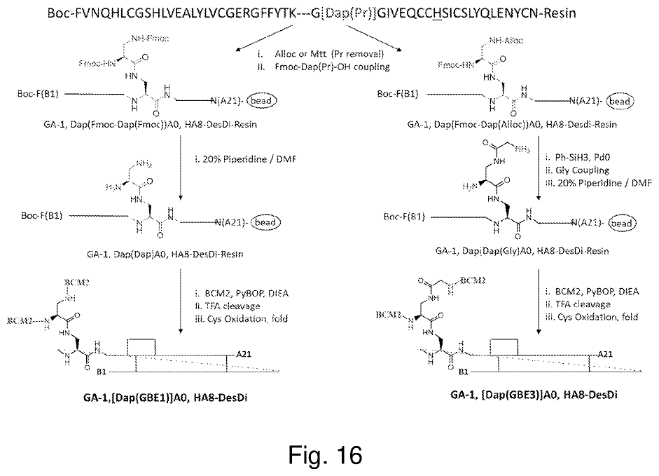

In addition, the modified A chain may also contain a broad molecular diversity of paired BCMs, either as branched adducts attached at one site of the modified A chain (e.g. at a single amino acid side chain) or as separate adducts attached at two or more peptide sites (e.g. at the side chain of two separate amino acids) as exemplified in FIG. 11A (far right panel). The paired boron-containing moieties of the present invention may thus contain combinations of individual BCMs such as phenylboronic acid (BCM1) elements and benzoboroxole (BCM2, BCM3) elements linked by a scaffold that is small relative to the size of insulin itself (<50 atoms) as illustrated in FIG. 8 (Branch-mer design), The BCM elements may contain halogen substituents to optimize its monosaccharide-binding properties at neutral pH. In the case of A chains modified at two or more sites, this scaffold would include intervening residues of the peptide as illustrated in FIG. 8 (tandem backbone design). The present invention thus encompasses novel combinations of (i) a modified A-chain peptide sequence, (ii) one or more attachment points at or near residue A1 and (iii) a set of boron-containing moieties (BCM where PBA and/or BXB are examples) such that such combinations provide an insulin-specific glucose-responsive or glucose-cleavable “switch.” Specifically, the molecular purpose of the paired boron-containing A-chain modifications is to provide a glucose-binding element capable of forming an intramolecular bond or bonds with B-chain-attached diol adducts such that the conformation of insulin is “closed” and so impaired in binding to the insulin receptor. Internal inter-chain tethering of the paired boron-containing elements thus recapitulates the inactive structure of a single-chain insulin analogue. In accordance with the present invention at high glucose concentrations the bond formed between the GBE and the diol adduct is broken due to competitive binding of the glucose to the paired BCMs of the GBE(s) (FIG. 6B). Preferred embodiments contain both of said paired BCMs and two or more diol groups in an effort to introduce cooperativity.

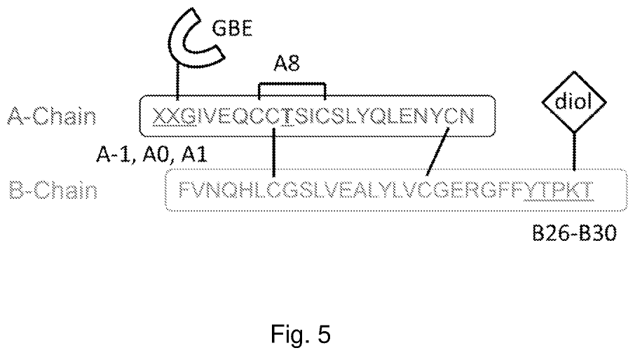

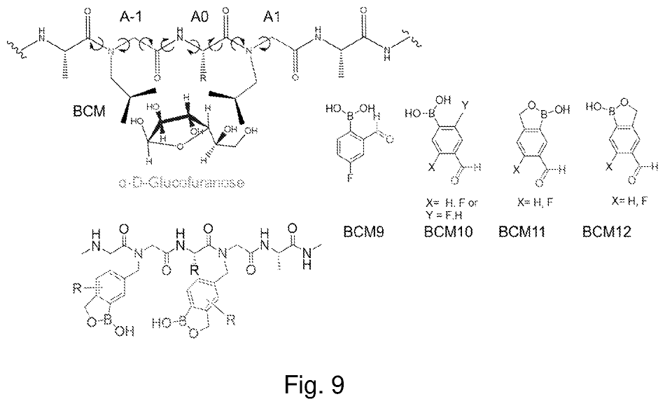

The GRI design related to the present invention considers three interlocking design elements required in concert to enable the function of a glucose-responsive conformational switch: element 1 is defined by the composition and chemical nature of the GBEs (e.g., mono-boronic acid, bis-boronic acid [with subclasses symmetrical and asymmetrical, each incorporating various BCMs as illustrated in FIGS. 8-11C), and intervening molecular connectivity, whether branched, attached to tandem main-chain sites in the A-chain peptide analogue, or other methods to tether two or more boron-containing moieties including reductive alkylation of peptide amide backbone nitrogen atoms using aldehyde containing BCMs (BCM9-BCM12; as illustrated in FIG. 9). In accordance with the present invention, such GBEs have dual roles to (i) competitively bind glucose while at the same time binding a B-chain-tethered diol or poly-ol (design element 2) and thereby (ii) in constrain an intramolecular switch (design element 3). Binding of the GBE to glucose competes with its binding to the internal diol component(s) (FIGS. 4 and 5).

Design element 2 is defined by the composition and chemical nature of B-chain-tethered diol moieties. The element functions as a glucose-competable “lock” to stabilize an inactive conformation of the hormone analogue. The modified A chains of the present invention can be combined with a combination of side-chain and main-chain diol modifications of the B chain at or near its C terminus as previously disclosed (USPTO Provisional Application 63/104,196, entitled “Molecular Designs of Glucose-Responsive and Glucose-Cleavable Insulin Analogues”; incorporated by reference herein).

Design element 3 specifies the placement of the intramolecular switch that effects a glucose-dependent conformational transition between active and inactive states. Optimal placement of the A-chain modifications depends on the location of the B-chain diol(s) and vice versa. The present invention focuses on the combination of (a) A-chain analogues modified by pairs of boron-containing moieties (BCMs) at or near its N terminus and (b) B-chain analogues modified by diol moieties at or near its C terminus. Such co-modifications permit a ligand-regulated reversible conformational cycle between active and inactive states of insulin, where the ligand is preferably glucose (FIGS. 5 and 6B). This scheme is in accordance with structural models of the complex between insulin and the insulin receptor as determined by X-ray crystallography and single-particle cryo-electron microscopic image reconstruction.

Although analogous glucose-displaceable bridges may be placed at other sites in the insulin molecule, the present scheme and its reverse (i.e., diol modification of the A chain at or near its N terminus with cognate modification of the B chain by pairs of boron-containing moieties, SEQ IDs offer the unique advantage of a closed state that mirrors that ultra-stable structure of a “mini-proinsulin” in which a direct peptide bond connects a C-terminal B chain residue (B28, B29, or B30) to GlyA1 or in which 1-3 intervening residues are placed between residues B30 and A1 as foreshortened connecting domains. Such tethering of the C terminus of the B chain to the N terminus of the A chain constrains conformational fluctuation otherwise associated with chemical and physical degradation of insulin in pharmaceutical formulations. Accordingly, applicant anticipates that, in addition to conferring the desired functional properties of a glucose-regulated switch in vivo, the present class of designs confers augmented shelf life of a pharmaceutical formulation in the absence of glucose or competing diol in the solution.

In accordance with one embodiment a novel design scheme is provided that reduces to practice a glucose-cleavable tether between the C terminus of the B chain and N terminus of the A chain, thereby providing a novel class of insulin analogues that would be closed and inactive at low glucose concentration but open and active at high glucose concentration. The resulting cycle of conformational states (FIG. 4) would in principle be reversible (depending on a patient's metabolic state), and its implementation would be consistent with the structure of the insulin-IR ectodomain complex. Sequence IDs, molecular compositions and protein sequences for specific and critical GBE-linked DOI intermediates and preferred GBE-containing K(DHBA)B28-KP GRIs are provided in the SEQ ID section. Table 1 contains a list of valuable on-resin synthetic intermediates, purified single-chain insulin analogues that are useful for further design and elaboration commensurate with the spirit of this invention, and two groups of putative GRIs designated as; A-Chain GBE, B-Chain (Diol)B28 Analogues (16 embodiments) and A-Chain DHBA, B-Chain (GBE)B28 Analogues (13 embodiments).

In accordance with another embodiment an insulin analogue is provided comprising a first and second glucose-binding element, wherein the first and second glucose-binding element each comprises a boron-containing diol-binding moiety. The insulin analogue comprises any of the known analogues of the native insulin A chain and B chain. In one embodiment an insulin A chain peptide and an insulin B chain peptide are provided, wherein said insulin A chain and B chain peptides are linked to each other by disulfide bonds, further wherein either
the insulin A chain peptide comprises the first and second glucose-binding elements covalently linked to one or more N-terminal amino acids located at positions selected from A₋₁, A₀, and A1; and
the insulin B chain peptide comprises a modified amino acid located at a position selected from B₂₃-B₃₁, said modified amino acid comprising a diol adduct; or
the insulin B chain peptide comprises said first and second glucose-binding elements covalently linked to one or more amino acids located at positions selected from B₂₆-B₃₂ (inclusive of a potential two-residue C-terminal extension); and
the insulin A chain peptide comprises a modified amino acid located at a position selected from A₋₁, A₀, and A₁, said modified amino acid comprising a diol adduct. In one embodiment the boron-containing diol-binding moiety of the first and second glucose-binding elements are independently selected from any of the boron-containing diol-binding moieties selected from the group consisting of BCM1-BCM12 as shown in FIG. 7.

In one embodiment the insulin analogue comprises a first and second glucose-binding elements linked to a first and second amino acid, respectively, of the insulin A chain peptide, wherein the first and second amino acids are located at positions i and i+1 or i and i+2 relative to each other. In one embodiment the first and second glucose-binding elements are bound to each other via a linker to form a complex, wherein the complex is covalently linked to a single amino acid of the insulin A chain peptide. In one embodiment the first and second glucose-binding elements are linked to the side chains of one or more amino acids of the insulin A chain peptide. In another embodiment the first and second glucose-binding elements are linked to the backbone amide nitrogens of one or more amino acids of the insulin A chain peptide. In one embodiment the first and second glucose-binding elements, each comprising a boron-containing diol-binding element, are covalently linked to the side chain of an amino acid selected from the group consisting of L-Cys, L-Lys, D-Lys, L-Orn, D-Orn, L-Dab, and D-Dab.

The analogues of the present invention may optionally contain an additional saccharide-binding element attached to residue B1 as a mechanism intended to provide glucose-sensitive binding of the insulin analogue to surface lectins in the subcutaneous depot. In addition, the analogues of the present invention may optionally contain substitutions known in the art to confer rapid action (such as Asp^{B28}, a substitution found in insulin aspart (the active component of Novolog®); [Lys^{B28}, Pro^{B29}], pairwise substitutions found in insulin lispro (the active component of Humalog®); Glu^{B29} or the combination [Lys^{B3}, Glu^{B29}] as the latter is found in insulin glulisine (the active component of Apridra®), or modifications at position B24 associated with accelerated disassembly of the insulin hexamer (e.g., substitution of Phe^{B24} by Cyclohexanylalanine or by a derivative of Phenylalanine containing a single halogen substitution within the aromatic ring). Alternatively, the analogues of the present invention may optionally contain modifications known in the art to confer protracted action, such as modification of the ε-amino group of Lys^{B29} by an acyl chain or acyl-glutamic acid adduct as respectively illustrated by insulin detemir (the active component of Levemir®) and insulin degludec (the active component of Tresiba®); or contain basic amino-acid substitutions or basic chain extensions designed to shift the isoelectric point (pI) to near neutrality as exemplified by the Arg^{B31}-Arg^{B32} extension of insulin glargine (the active component of Lantus®). Analogues of the present invention designed to exhibit such a shifted pI may also contain a substitution of Asn^{A21}, such as by Glycine, Alanine or Serine. Analogues of the present invention may optionally also contain non-beta-branched amino-acid substitutions of Thr^{A8} associated with increased affinity for the insulin receptor and/or increased thermodynamic stability as may be introduced to mitigate deleterious effects of the primary two above design elements (a phenylboronic acid derivative at or near the N-terminus of the A chain and one or more saccharide derivatives at or near the C-terminus of the B chain) on receptor-binding affinity and/or thermodynamic stability. Examples of such A8 substitutions known in the art are His^{A8}, Lys^{A8}, Arg^{A8}, and Glu^{A8}.

The insulin analogues of the present invention may exhibit an isoelectric point (pI) in the range 4.0-6.0 and thereby be amenable to pharmaceutical formulation in the pH range 6.8-7.8; alternatively, the analogues of the present invention may exhibit an isoelectric point in the range 6.8-7.8 and thereby be amenable to pharmaceutical formulation in the pH range 4.0-4.2. The latter conditions are known in the art to lead to isoelectric precipitation of such a pI-shifted insulin analogue in the subcutaneous depot as a mechanism of protracted action. An example of such a pI-shifted insulin analogue is provided by insulin glargine, in which a basic two-residue extension of the B chain (Arg^{B31}-Arg^{B32}) shifts the pI to near-neutrality and thus enables prolonged pharmacokinetic absorption from the subcutaneous depot. In general, the pI of an insulin analogue may be modified through the addition of basic or acidic chain extensions, through the substitution of basic residues by neutral or acidic residues, and through the substitution of acidic residues by neutral or basic residues; in this context we define acidic residues as Aspartic Acid and Glutamic Acid, and we define basic residues as Arginine, Lysine, and under some circumstances, Histidine. We further define a “neutral” residue in relation to the net charge of the side chain at neutral pH.

It is an additional aspect of the present invention that absolute in vitro affinities of the insulin analogue for insulin receptor (isoforms IR-A and IR-B) are in the range 5-100% relative to wild-type human insulin and so unlikely to exhibit prolonged residence times in the hormone-receptor complex; such prolonged residence times are believed to be associated with enhanced risk of carcinogenesis in mammals or more rapid growth of cancer cell lines in culture. It is yet an additional aspect of the present invention that absolute in vitro affinities of the insulin analogue for the Type 1 insulin-like growth factor receptor (IGF-1R) are in the range 5-100% relative to wild-type human insulin and so unlikely either to exhibit prolonged residence times in the hormone/IGF-1R complex or to mediate IGF-1R-related mitogenesis in excess of that mediated by wild-type human insulin.

The insulin analogues of the present invention consist of two polypeptide chains that contain a novel paired PBA-PBA, PBA-BXB, BXB-PBA and/or BXB-BXB modifications in the A chain such that the analogue, in the absence of glucose or other exogenous saccharide, contains covalent bonds the paired boron-containing elements and diol adducts in the B chain. Although we do not wish to be restricted by theory, we envisage that these two design elements form a covalent interaction in the absence of exogenous glucose such that the structure of the hormone is stabilized in a less active conformation. Two alternative design schemes are envisioned that would follow the same principles to provide a glucose-responsive insulin. The first switches the positions of the glucose-binding elements and diol modifications such that the former are attached at or near the C terminus of the B chain whereas the latter are attached at or near the N terminus of the A chain. The second embodiment replaces diol modifications by glucose-binding elements such that both chains are modified by pairs of boron-containing moieties.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We envisage that such an insulin analogue formulation would be compatible with multiple devices (such as insulin vials, insulin pens, and insulin pumps) and could be integrated with modifications to the insulin molecule known in the art to confer rapid-, intermediate-, or prolonged insulin action. In addition, the present glucose-regulated conformational switch in the insulin molecule, engineered between the C-terminus of the B chain and N-terminus of the A chain, could be combined with other glucose-responsive technologies (such as closed-loop systems or glucose-responsive polymers) to optimize their integrated properties. We thus envisage that the products of the present invention will benefit patients with either Type 1 or Type 2 diabetes mellitus both in Western societies and in the developing world.

In one embodiment a method of treating a diabetic patient while decreasing the risk of hypoglycemia is provided. In accordance with the present disclosure the method comprising administering a physiologically effective amount of any of the insulin analogues disclosed herein that comprise a glucose-binding elements, or a physiologically acceptable salt thereof, to the patient.

In one embodiment a glucose responsive insulin analogue is provided wherein the insulin analogue comprises
a first and second glucose-binding element, said first and second glucose-binding element each comprising a boron-containing diol-binding moiety;
an insulin A chain peptide;
an insulin B chain peptide; wherein said insulin A and B chain peptides are linked to each other by disulfide bonds; further wherein either
I. said insulin A chain peptide comprises said first and second glucose-binding elements covalently linked to one or more N-terminal amino acids located at positions selected from A₋₁, A₀, and A1; and said insulin B chain peptide comprises a modified amino acid located at a position selected from B₂₃-B₃₁, said modified amino acid comprising a diol adduct; or
II. said insulin B chain peptide comprises said first and second glucose-binding elements covalently linked to one or more amino acids located at positions selected from B₂₃-B₃₁; and said insulin A chain peptide comprises a modified amino acid located at a position selected from A₋₁, A₀, and A₁, said modified amino acid comprising a diol adduct.

In one embodiment the boron-containing diol-binding moiety of said first and second glucose-binding elements of the insulin analogue are independently selected from the group consisting of BCM1-BCM12 as shown in FIG. 7, optionally wherein the boron-containing diol-binding moiety is phenylboronic acid or benzoboroxole. In one embodiment the first and second glucose-binding elements are covalently linked to a first and second amino acid, respectively, of said insulin A chain peptide, wherein the first and second amino acids are located at positions i and i+1 or i and i+2 relative to each other. In one embodiment the first and second glucose-binding elements are bound to each other via a linker to form a complex, wherein the complex is covalently linked to a single amino acid. In one embodiment the first and second glucose-binding elements are covalently linked to amino acids located at a position selected from A₋₁, A₀ and A₁ and one or more a diol adducts are linked to an amino acid located at a position selected from B₂₈ or B₂₉. In one embodiment the first and second glucose-binding elements are covalently linked to the side chain or the or backbone amide of the insulin A chain peptide, optionally wherein the first and second glucose-binding elements are covalently linked to the side chain of an amino acid selected from the group consisting of L-CyS, L-Lys, D-Lys, L-Orn, D-Orn, L-Dab and D-Dab. In one embodiment the amino acid comprising a diol adduct has the structure of

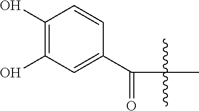 [SMILES: *C(=O)c1ccc(O)c(O)c1]

linked to the side chain or backbone amide of the insulin B chain peptide. In one embodiment the first and second glucose-binding elements are bound to each other via a linker to form a complex having the general structure

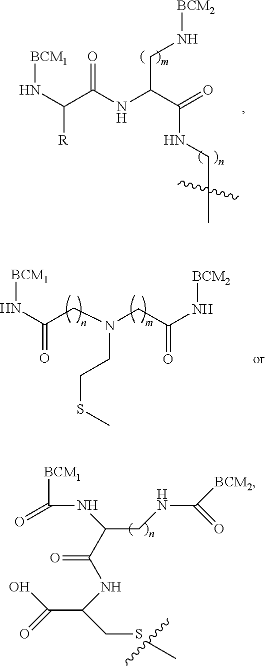

wherein BCM₁ and BCM₂ are boron-containing moieties; n and m are independently an integer selected from the range of 1 to 3;
R is any amino acid side chain of the L or D configurations of the standard 20 proteogenic amino acids or non-proteogenic amino acids known in the art. In one embodiment R is selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O. In one embodiment R is H, C₁-C₆ alkyl, (C₁-C₄ alkyl)C(O)NH₂, (C₁-C₄ alkyl)OH, (C₁-C₄ alkyl)CH₃OH, (C₁-C₄ alkyl)S, (C₁-C₄ alkyl)SCH₃, (C₁-C₄ alkyl)COOH and (C₁-C₄ alkyl)NH₂,

In one embodiment a glucose-sensing insulin analogue is provided wherein the insulin A chain peptide
comprises a sequence of
GX₀GIVEQX₆CX₈SIX₁₁SLYQLENYCX₂₁ (SEQ ID NO: 13);
X₀GIVEQX₆CX₈SIX₁₁SLYQLENYCX₂₁ (SEQ ID NO: 14);
GX₁IVEQX₆CX₈SIX₁₁SLYQLENYCX₂₁ (SEQ ID NO: 15); or
X₁IVEQX₆CX₈SIX₁₁SLYQLENYCX₂₁ (SEQ ID NO: 16), and the insulin B chain peptide comprises a sequence of
   FVX₂₃QX₂₅LCGX₂₉X₃₀LVEALYLVCGERGFF-R₂₃ (SEQ ID NO: 17), wherein X₀ and X₁ are independently a modified amino acid selected from D-Cys, L-Asp, D-Asp, D-Glu, L-Glu, D-homocys, L-homocys, L-Dap, D-Dap L-Cys, L-Lys, D-Lys, L-Orn, D-Orn, L-Dab, and D-Dab, wherein the modified amino acid comprises the first and second glucose-binding elements linked to the side chain or the backbone amide of the modified amino acid. In one embodiment X₀ is a modified L-Cys, L-Lys, D-Lys, L-Orn, D-Orn, L-Dab, and D-Dab, wherein the modified amino acid comprises a glucose-binding elements linked to the side chain of the amino acid. X₆ and X₁₁ are independently Cys or Selenocysteine; X₈ is selected from the group consisting of threonine, lysine, arginine, glutamic acid, and histidine; X₂₁ is selected from the group consisting of asparagine, serine, glycine and alanine; X₂₃ is asparagine or lysine; X₂₅ is selected from the group consisting of histidine and threonine; X₂₉ is selected from the group consisting of alanine, glycine and serine; X₃₀ is selected from the group consisting of histidine, aspartic acid, glutamic acid, homocysteic acid and cysteic acid; and R₂₃ is selected from the group consisting of YTX₂₈KT (SEQ ID NO: 18), YTX₂₈KP (SEQ ID NO: 19), YTKPT (SEQ ID NO: 20), YTKPTR (SEQ ID NO: 21), YTKPTRR (SEQ ID NO: 22), YTX₂₈K (SEQ ID NO: 23), YTKP (SEQ ID NO: 24), YTPK (SEQ ID NO: 25), YTX₂s, YT, and Y; wherein X₂₈ is proline, aspartic acid or glutamic acid, and the side chain the or backbone amide of an amino acid at the C-terminus of the B chain is modified to comprise a diol adduct.

In one embodiment the insulin analog comprises an insulin A chain peptide comprising a sequence of GX₀GIVEQX₆CX₈SIX₁₁SLYQLENYCX₂₁ (SEQ ID NO: 13);
X₀GIVEQX₆CX₈SIX₁₁SLYQLENYCX₂₁ (SEQ ID NO: 14);
GX₁IVEQX₆CX₈SIX₁₁SLYQLENYCX₂₁ (SEQ ID NO: 15); or
X₁IVEQX₆CX₈SIX₁₁SLYQLENYCX₂₁ (SEQ ID NO: 16), and an insulin B chain peptide comprising a sequence of
   FVX₂₃QHLCGSHLVEALYLVCGERGFFYTX₂₈KP (SEQ ID NO: 26), wherein X₀ and X₁ are independently a modified amino acid selected from L-Cys, L-Lys, D-Lys, L-Orn, D-Orn, L-Dab, and D-Dab, wherein the modified amino acid comprises said first and second glucose-binding elements linked to the side chain or the or backbone amide of the modified amino acid; X₆ and Xn₁ are independently Cys or Selenocysteine; X₈ is selected from the group consisting of threonine, lysine, arginine, glutamic acid, and histidine; X₂₁ is selected from the group consisting of asparagine, serine, glycine and alanine; X₂₃ is asparagine or lysine; and X₂₈ is proline, aspartic acid or glutamic acid, and wherein the side chain or backbone amide of an amino acid at the C-terminus of the B chain is modified to comprise a diol adduct.

In one embodiment the glucose sensing insulin analog comprises an insulin A chain peptide comprising a sequence of GIVEQCCX₈SICSLYQLENYCX₂₁ (SEQ ID NO: 27); and said B chain comprises the sequence the sequence FVKQX₂₅LCGSHLVEALYLVCGERGFFYTEKT (SEQ ID NO: 28), FVNQX₂₅LCGSHLVEALYLVCGERGFFYTDKT (SEQ ID NO: 29), FVNQX₂₅LCGSHLVEALYLVCGERGFFYTKPT (SEQ ID NO: 30), FVNQHLCGSHLVEALYLVCGERGFFYTKKP (SEQ ID NO: 31) or FVNQX₂₅LCGSHLVEALYLVCGERGFFYTPKT (SEQ ID NO: 32) wherein X₈ is selected from the group consisting of threonine and histidine; X₂₁ is selected from the group consisting of asparagine, glycine and alanine; X₂₅ is selected from the group consisting of histidine and threonine, and a lysine at position 28 or 29 of said B chain has been modified to comprise a diol adduct.

In accordance with one embodiment any of the glucose-sensing insulin analogues disclosed herein can be provided with two or more glucose-binding moieties that comprise the same boron-containing diol-binding motif. In one embodiment the boron-containing diol-binding moiety is phenylboronic acid, a halogen-modified fluorophenylboronic acid or benzoboroxole. The glucose-binding elements of the present invention contain two or more boron atoms, and so this encompasses pairs, triplets or high-order combinations of single boron-containing moieties (such as PBA and Bxb); these can be respectively attached to different amino acids selected from positions A₋₁, A₀ and/or A₁; or they can be moieties within a single complex adduct attached to only one of these peptide sites (see FIG. 11A, right).

### Example 1

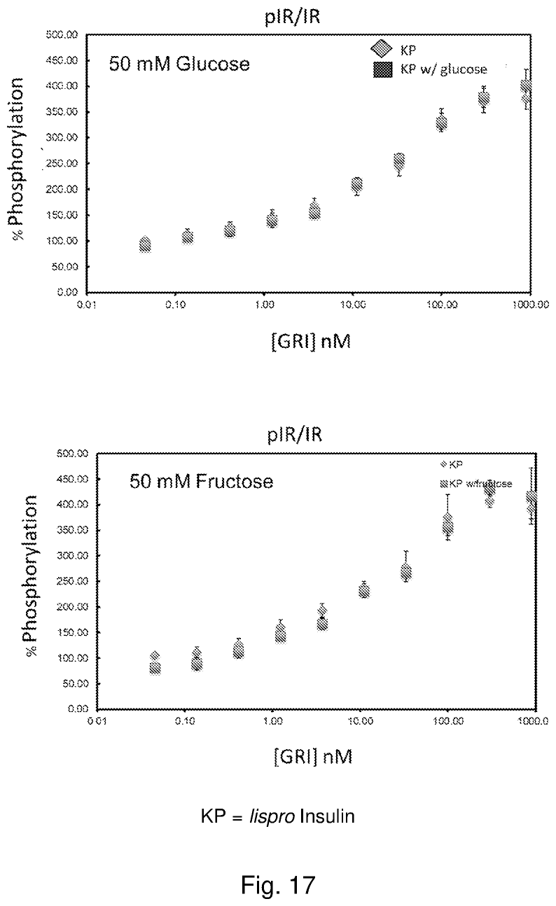

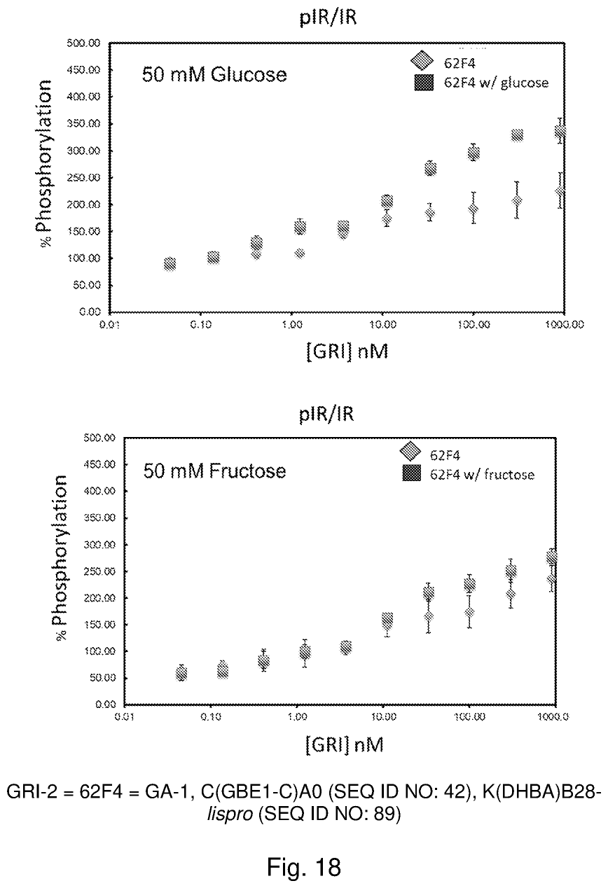

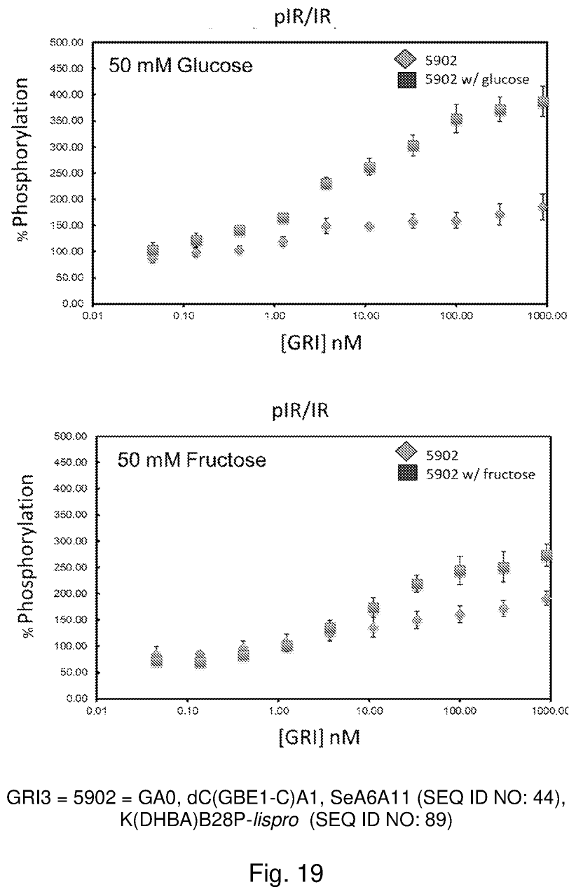

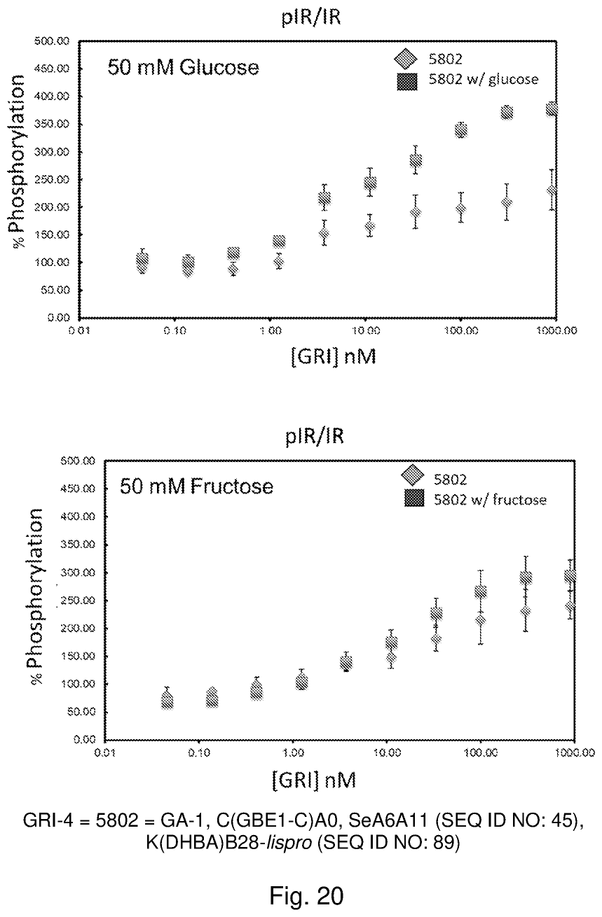

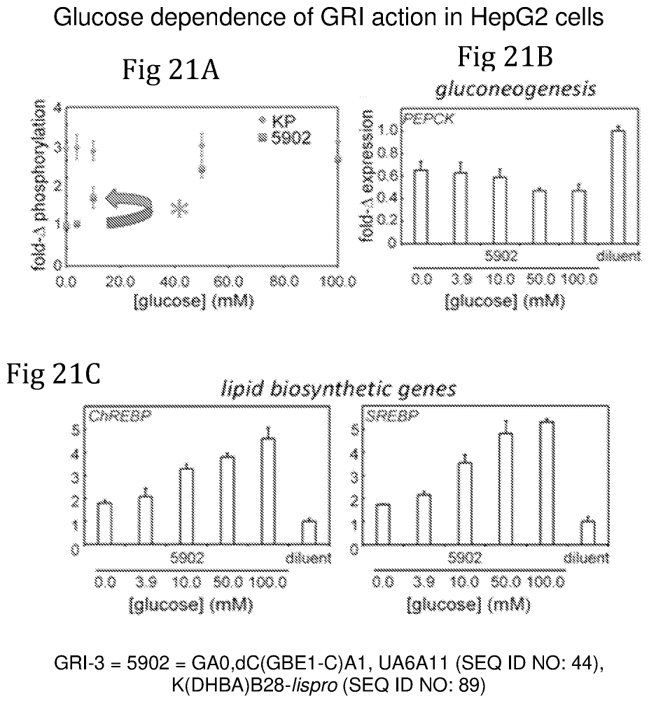

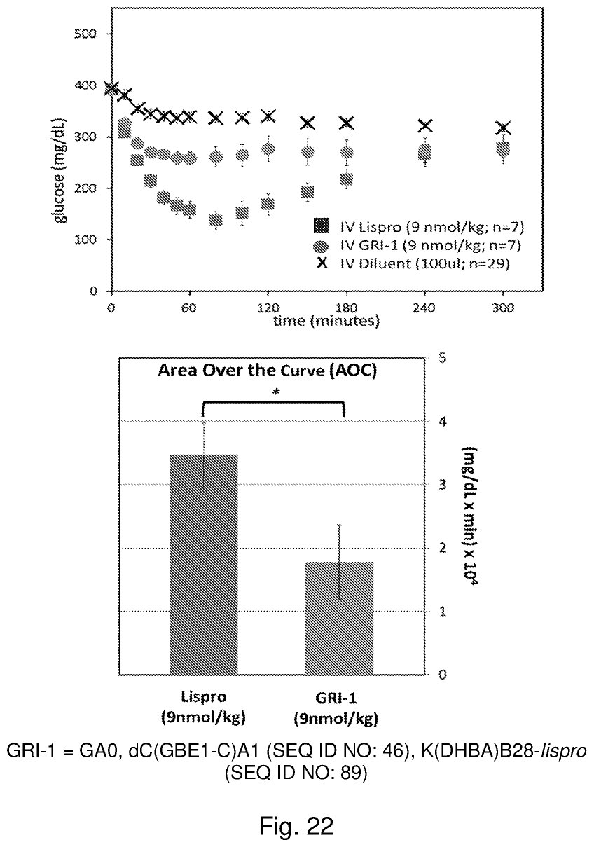

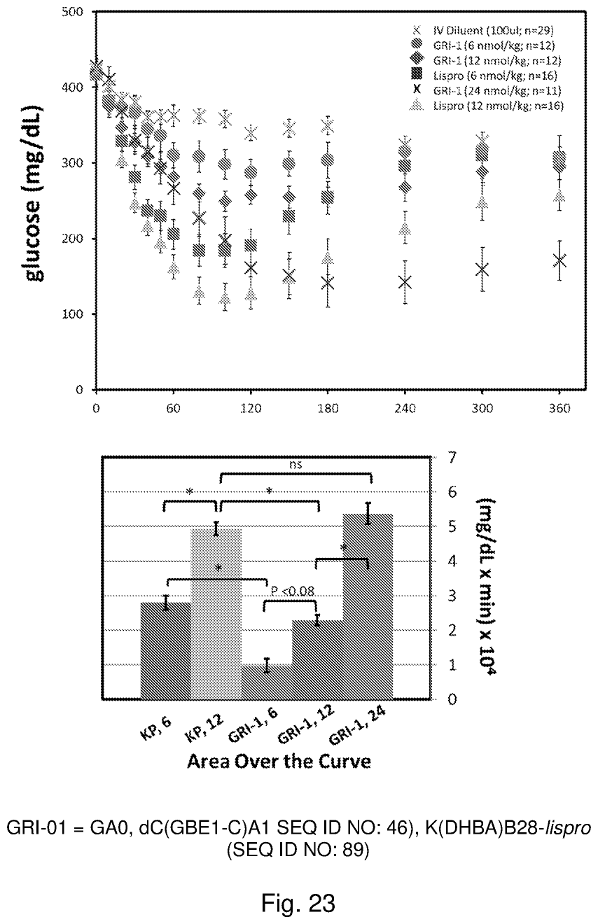

Representative examples of the glucose-responsive insulin analogues of the present invention were prepared for functional testing in mammalian cell culture in the context of insulin lispro such that the epsilon-amino group of LysB28 was modified by 2, 3-dihydroxybenzoic acid (DHBA), thereby providing a diol at or near the C terminus of the B chain. The A chain was modified by a D-Cysteine at position A1 by a symmetrical pair of benzoboroxole moieties as known in the art to bind diols, including glucose and less favorably fructose. The glucose-dependence and fructose-dependence of biological activity were measured in human liver-derived HepG2 cells in culture. Whereas the parent analogue insulin lispro exhibited no changes in biological activity on addition of either monosaccharide (FIG. 17), the insulin analogues of the present invention exhibited the targeted glucose dependence (in upper panels found in FIGS. 19-21) and to a less marked degree, fructose dependence (in lower panels found in FIGS. 19-21). The range of effective glucose concentration was in accordance with therapeutic goals for the treatment of diabetes mellitus (FIG. 21).

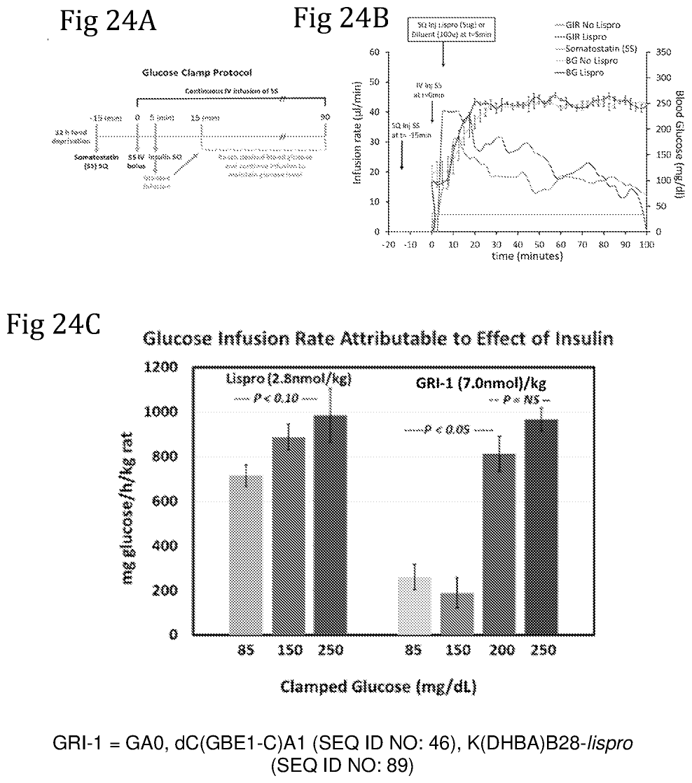

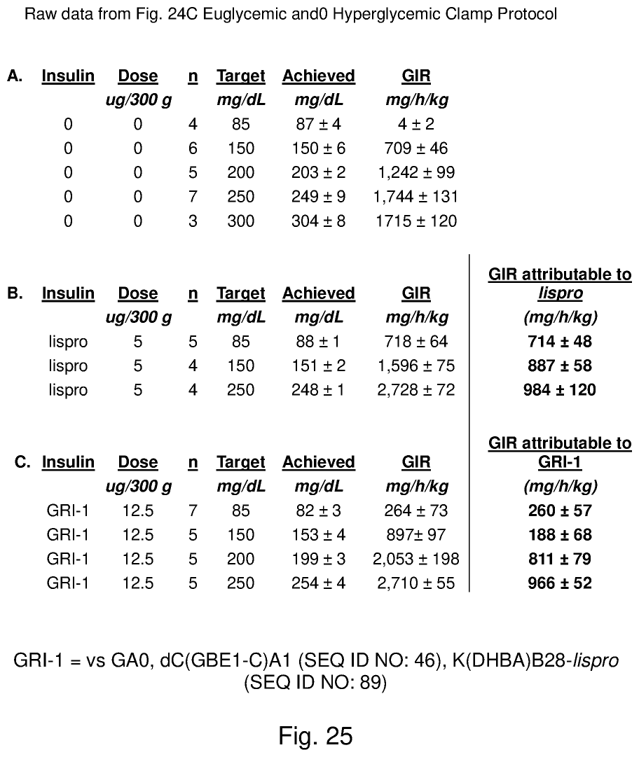

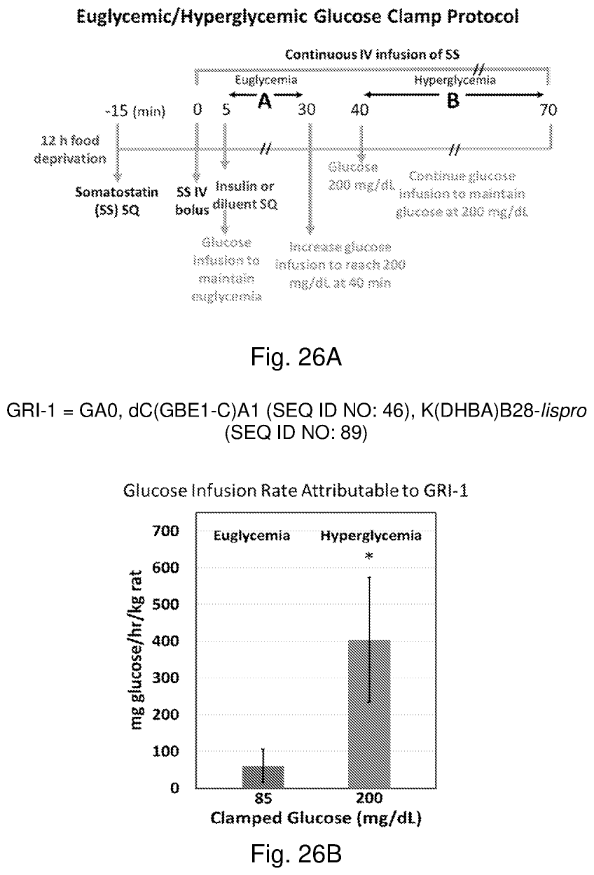

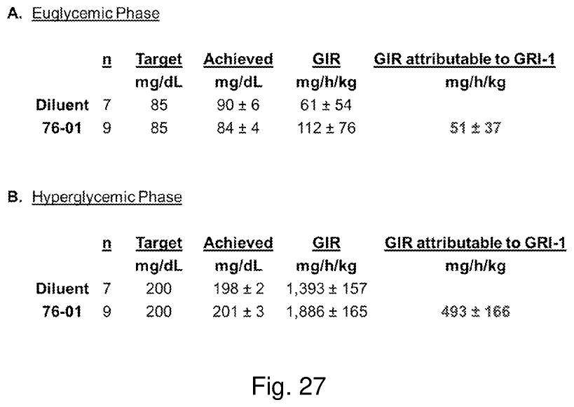

We note in passing that strengths of clinical insulin formulations are measured not in molar concentrations, but instead in relation to biological potency in a standard test animal (usually rabbits). For example, Levemir contains four times the hormone concentration in molar units as other clinical insulin products, but is nonetheless labeled as “U-100” in strength. This is because the per molecule activity of insulin detemir (the active component of Levemir) is fourfold reduced relative to human insulin. By the same token, the per-molecule activity of the present insulin analogues are typically reduced by the A-chain modifications or by the B-chain modifications. U-100 strength can nonetheless be achieved by increasing the analogue's protein concentration by analogy to Levemir. The example of GRI-1 highlighted its Levemir-like reduction in per-molecule potency, whose precise value was observed to depend on the blood-glucose concentration. Thus, experimental design exploited prior baseline studies of intrinsic potency to equalize activities rather than to equalize molar concentrations (see FIGS. 24A-24C).

Control studies of “partial analogues” (i.e., containing only an A-chain-linked GBE or only a B-chain-linked diol moiety) were undertaken to test whether both components of the designed switch would be required for (a) a change in protein conformation or (b) glucose-dependent activation of biological activity. These controls were analogous to those described by Chen, Y.-S., et al. (2021) in the case of a fructose-responsive insulin (FRI). Protein conformation and glucose binding were monitored by ¹H-¹³C HSQC NMR spectra. Glucose-dependent activities were assessed in normal rats versus streptozotocin (STZ)-induced diabetic rats. These control data validated that, as in the FRI, GRI-1 requires both components of the switch in the same protein molecule as envisioned in our design scheme. Neither “partial analogue” exhibited GRI-like functional properties.

The closed-open transition of the present GRI analogues was further validated by cryo-EM single-particle image reconstruction employing the isolated ectodomain of the human insulin receptor. Complexes were made either in the absence of glucose or in the presence of 50 mM glucose. Whereas addition of glucose led to an ectodomain-hormone structure essentially identical to that of the wild-type complex in its signaling conformation (Weis, F., et al. The signalling conformation of the insulin receptor ectodomain. Nature Communications 9(1), 4420 (2018)), in the absence of glucose the structure was of lower resolution and without binding of the insulin analogue to “Site 1” of the receptor; the overall ectodomain conformation was not in the active, signaling state. Thus, the predicted mechanism underlying the present class of GRI analogues was explicitly visualized in these studies.

### Example 2

Another, related prototype GRI (designated GRI-2) was prepared that differed from GRI-1 in one respect: the same dual-boron-containing GBE (GBE1-C) was attached to L-CysA₀; the diol-modified Lysine at B28 was retained. GRI-5 was tested in HepG2 cells in culture (as described in Chen, Y.-S., et al. (2021) and observed to exhibit glucose-dependent activity.

### Example 3

Yet another related prototype GRI (designated GRI-3) was prepared that differed from GRI-1 in only by substitution of cystine A6-A11 by a diselenide bridge (SecA6 and SecA11). This modification stabilizes insulin as described by Weil-Ktorza, O., et al. (Substitution of an Internal Disulfide Bridge with a Diselenide Enhances both Foldability and Stability of Human Insulin. Chemistry: a European Journal 25(36), 8513-21 (2019)). GRI-3 was tested in HepG2 cells in culture (as described in Chen, Y.-S., et al. (2021) and observed to exhibit glucose-dependent activity.

### Example 4

Yet another related prototype GRI (designated GRI-4) was prepared that differed from GRI-2 in only by substitution of cystine A6-A11 by a diselenide bridge (SecA6 and SecA11). This modification stabilizes insulin as described by Weil-Ktorza, O., et al. (Substitution of an Internal Disulfide Bridge with a Diselenide Enhances both Foldability and Stability of Human Insulin. Chemistry: a European Journal 25(36), 8513-21 (2019)). GRI-4 was tested in HepG2 cells in culture (as described in Chen, Y.-S., et al. (2021) and observed to exhibit glucose-dependent activity.

### Example 5

A related prototype GRI (designated GRI-5) was prepared that differed from GRI-1 in two respects: (i) the aromatic diol moiety (DHBA) was attached to an Ornithine side chain at position B28, as substituted for LysB28 in insulin lispro; and (ii) the same dual-boron-containing moiety (GBE1-C) was attached to L-CysA₀ instead of D-CysA1 as in GRI-1. GRI-5 was tested in STZ rats and in normal rats as an initial screen for glucose-dependent biological activity. Its functional properties in these animal models were similar to those of GRI-1.

### Example 6

A fructose-responsive insulin (FRI) was prepared to demonstrate proof of principle as described in Chen, Y.-S., et al. (2021) and incorporated by reference herein. This provided a model for Examples 1-5 above.

A switchable insulin analogue (designated FRI; fructose-responsive insulin) contains meta-fluoro-PBA* (meta-fPBA or m-fPBA) as a diol sensor linked to the α-amino group of Gly^{A1} and an aromatic diol (3,4-dihydroxybenzoic acid; DHBA) attached to the e-amino group of Lys^{B28} of insulin lispro. Although fructose and glucose each contain diols, the sensor preferentially binds to aligned 1,2-diol groups as found in β-D-fructofuranose and α-D-glucofuranose. Affinity of meta-fPBA is higher for fructose than glucose due to salient differences in respective conformational; binding is covalent but reversible. To compensate for impairment of IR-binding affinity generally associated with N-linked adducts at Gly^{A1}, Thr^{A8} was substituted by His a favorable substitution found in avian insulins. Control analogues were provided by 1) insulin KP, 2) a KP derivative containing an A1-linked meta-fPBA but not the B28 diol (diol-free control; DFC), and 3) a peptide bond between Lys^{B28} and Gly^{A1} in a des-[B29, B30] (“DOI”) template. The latter [a covalent “closed” state] was inactive.

Western Blot Assays Demonstrated Fructose-Dependent Signaling. Structural studies suggest that insulin's hinge-opening at a dimer-related αCT/L1 interface is coupled to closure of IR ectodomain legs, leading to TK-mediated trans-phosphorylation and receptor activation. Signal propagation was probed via a cytoplasmic kinase cascade and changes in metabolic gene expression in HepG2 cells. Control studies indicated that addition of 0 to 100 mM fructose or glucose did not trigger changes in signaling outputs. An overview of IR autophosphorylation (probed by anti-pTyr IR antibodies) and downstream phosphorylation of Ser-Thr protein kinase AKT (protein kinase B; ratio p-AKT/AKT), forkhead transcription factor 1 (p-FOXO1/FOXO1), and glycogen synthase kinase-3 (p-GSK-3/GSK-3) at a single hormone dose (50 nM) was provided by Western blot (WB). In each case, WBs demonstrated fructose-dependent signaling by FRI and fructose-independent signaling by KP and DFC. The activity of FRI in the absence of fructose is low.

Plate Assays Demonstrated Ligand-Selective Signaling. Quantitative dose-dependent and ligand-selective IR autophosphorylation were evaluated in a 96-well plate assay. FRI triggered a robust signal on addition of 50 mM fructose whereas baseline activity in the absence of fructose was low. As expected, KP and DFC exhibited high signaling activity in the presence or absence of fructose, respectively). Ligand-dependent activation of FRI is specific to fructose as addition of 50 mM glucose did not influence its activity (nor the activities of KP and DFC). These data indicate that in 50 mM fructose FRI is almost as active as KP.

PCR Assays Demonstrated Ligand-Selective Metabolic Gene Regulation. Insulin-signaling in hepatocytes extends to metabolic transcriptional regulation as recapitulated in HepG2 cells. At hypoglycemic conditions, the cells exhibited stronger gluconeogenesis-related responses following insulin stimulation than at hyperglycemic conditions. In this protocol, FRI, when activated by fructose, regulated downstream expression of the gene encoding phosphoenolpyruvate carboxykinase (PEPCK; a marker for hormonal control of gluconeogenesis). Under normoglycemic conditions, FRI, when activated by fructose, regulated the genes encoding carbohydrate-response-element and sterol response-element binding proteins (ChREBP and SREBP; markers for hormonal control of lipid biosynthesis). No fructose dependence was observed in control studies of KP and DFC; no effects were observed on addition of glucose instead of fructose. Control studies were undertaken in the absence of insulin analogues to assess potential confounding changes in metabolic gene expression on addition of 0 to 100 mM fructose or 0 to 100 mM glucose. No significant effects were observed in either case, indicating that the present short-term fructose exposure (to activate FRI) is unassociated with the transcriptional signature of longer-term exposure.

Ligand-Binding to FRI Affects Protein Structure. Far-UV circular dichroism (CD) spectra of FRI and DFC are indistinguishable from parent analogue insulin lispro (KP), indicating that secondary structure is not affected by the modifications at A1 and B28. Difference CD spectra calculated on addition of 100 mM fructose or glucose were in each case featureless. High-resolution NMR spectroscopy [as enabled by the monomeric KP template] corroborate essential elements of the intended fructose-selective switch

¹⁹F-NMR spectra monitored fructose sensor. The fluorine atom in meta-fPBA provided an NMR-active nucleus. Addition of 0 to 100 mM fructose led to an upfield change in 19F-NMR chemical shift in slow exchange on the NMR time scale. This upfield shift presumably reflects displacement of an aromatic diol by a nonaromatic ligand. No change in FRI ¹⁹F chemical shift was observed on addition of glucose. Although an analogous ¹⁹F resonance was observed in the NMR spectrum of DFC, its chemical shift did not change on addition of glucose or fructose. Interestingly, a broadened ¹⁹F signal was observed in ligand-free DFC, probably due to conformational exchange or self-association; this signal sharpened on addition of ligand (fructose or glucose).

Dual ¹⁹F- and ¹H NMR-monitored titration and natural-abundance ¹H-¹³C heteronuclear single quantum coherence (HSQC) spectra provided further evidence of a specific interaction between FRI and fructose.

¹H-¹³C 2D HSQC spectra monitored “closed” conformation of ligand-free FRI. One-dimensional (1D) ¹H and ¹H-¹³C HSQC spectra of DFC were similar to those of parent analogue insulin lispro (KP), excepting methyl resonances of IleA2 and ValA3 (adjacent to the Gly^{A1}-attached meta-fPBA). Patterns of ¹H-¹³C chemical shifts of FRI and DFC were also similar. Those NMR features provided evidence that FRI and DFC retain a native-like structure. However, in FRI, the resonances of Ile^{A2}, Val^{A3}, Leu^{B11}, Val^{B12}, and Leu^{B15} exhibited larger chemical shift differences (relative to KP) than in DFC. These findings suggest that FRI exhibits a local change in conformation and/or dynamics, presumably due to the intended DHBA/meta-fPBA tether. We envision that constraining the C-terminal B-chain segment alters aromatic ring currents affecting the central B-chain α-helix (via Tyr^{B26}-Leu^{B11}, Tyr^{B26}-Val^{B12}, and Phe^{B24}-Leu^{B15} packing) and N-terminal A-chain helix (via native-like TyrB26-IleA2 and Tyr^{B26}-Val^{A3} packing).

Aromatic ¹H-¹³C two-dimensional (2D) HSQC spectra monitor hinge-opening. ¹H-¹³C HSQC spectra provide probes of aromatic resonances in FRI's DHBA/meta-fPBA adducts in the absence of fructose and in the presence of 100 mM fructose. Significant chemical shift changes in both ¹H/¹³C dimensions were observed. Resonance assignments were corroborated by model studies of meta-fPBA- and DHBA-modified peptides. DHBA chemical shifts in fructose-free FRI are similar to those in the complex of model peptides, whereas such chemical shifts in fructose bound FRI are similar to that of free DHBA-modified octapeptide. In addition, methyl resonances sensitive to addition of fructose exhibited a trend toward corresponding chemical shifts observed in spectra of insulin lispro and ligand-free DFC. Together, these NMR features provide evidence that in FRI the Lys^{B28}-attached DHBA binds Gly^{A1}-linked meta-fPBA in absence of fructose, but this tether is displaceable by fructose.

Methyl ¹H-¹³C 2D HSQC spectra monitor protein core. Aliphatic ¹H-¹³C spectra reflect tertiary structure as probed by upfield-shifted methyl resonances. Changes in cross-peak chemical shifts were observed in FRI on overlay of spectra acquired in the absence of an added monosaccharide or on addition of 100 mM fructose. Fructose-binding accentuated upfield ¹H secondary shifts with smaller changes in ¹³C chemical shifts. These changes presumably reflect altered aromatic ring currents within insulin's core. Control studies of DFC suggested that such chemical shift changes require the interchain DHBA/meta-fPBA tether; in these spectra, changes were restricted to IleA2 immediately adjoining the sensor. Addition of 50 mM glucose caused essentially no changes in ¹H-¹³C fingerprints of FRI or DFC in accordance with the fructose selectivity of meta-fPBA.

### EXPERIMENTAL PROCEDURES

Methods pertaining to these assays, in addition to those described above, are as follows.

Chemical Synthesis of Single-Chain Insulin Precursors (SCIs) and Analogues. We employed solid-phase peptide synthesis to prepare an extensive collection of single-chain insulin precursors (Table 1). The peptides were synthesized starting with Pre-loaded Fmoc-Asn(Trt)-HMBA-CM resins using traditional Fmoc/tBu chemistry with repetitive DIC/6-Cl-HOBt activation/coupling cycles using DIC/6-Cl-HOBt activation (10 Equivalents) and IR or induction heating at 60° C. for 10 min per cycle and 50° C. for Fmoc deprotection (20% piperidine/DMF, 2×5 min). Tribute or Chorus automated peptide synthesizers (Gyros Protein Technology, Tucson, AZ) were used. All amino acids, DIC and 6-Cl-HOBt were purchased from Gyros Protein Technology (Tucson, AZ) or ChemImpex (Chicago, Ill). Peptides were cleaved from resin and deprotected by treatment with trifluoroacetic acid (TFA) containing 2.5% triisopropylsilane (TIS), 2.5% water, 2.5% DODT (ethylenedioxy)-diethenethiol), and 2.5% of Anisole for 3-4 hr.

SCIs were chemically synthesized using Fmoc/OtBu solid-phase chemistry on a Pre-loaded H-Asn(Trt)-HMBP-Chemmatrix resin, with repetitive coupling cycles using DIC/6-Cl-HOBt or DIC/Oxyma Pure (Ethyl cyano(hydroxyimino)acetate) activation (10 Equivalents) and IR or induction heating at 60° C. for 10 min per cycle and 50° C. for Fmoc deprotection (20% piperidine/DMF, 2×5 min). Tribute or Chorus automated peptide synthesizers (Gyros Protein Technology, Tucson, AZ) were used. Pre-loaded Fmoc-Asn(Trt)-Chemmatrix resin was used. All amino acids, DIC and 6-Cl-HOBt and Oxyma Pure were purchased from Gyros Protein Technology (Tucson, AZ). The peptide was cleaved and deprotected by treatment with trifluoroacetic acid (TFA) containing 2.5% triisopropylsilane (TIS), 2.5% water, 2.5% DODT (ethylenedioxy)-diethenethiol), and 2.5% of anisole. Peptides containing Sec(Mob) were cleaved in presence of 2,2′-dithio-bis-(5-nitropyridine) (DTNP, 2 equivalents per Sec; see K. M. Harris, S. Flemer Jr, R. J. Hondal, J. Pept. Sci. 2007, 13, 81-93). Cleavage mixture was precipitated with ether (5-10 fold with respect to TFA) and solid was isolated by centrifugation. Precipitate was further washed with twice with ether and dried in vacuo.

Oxidative folding of Single-chain Insulins. Crude linear reduced insulin precipitated and dried (in vacuo) were suspended and air oxidized at 0.1 mM in a folding buffer (Cys, 2.0 mM, Gly 20 mM, pH adjusted to 10.5 with NaOH (10 M)) with vigorous stirring for 17-48 h at 10° C. The reaction was monitor by RP-HPLC and LC-MS for reaction completion. Preparative HPLC was carried out using a C8 column was used for purification. Crude reaction was acidified (HCl, 5M to pH 3) filtered (0.2 μM) and then purified identity of the SCI was confirmed by LC-MS (Finnigan LCQ Advantage, Thermo) on a TARGA C8 (4.6×250 mm, 5 μm, Higgins Analytical) with 0.1% TFA/H₂O (A) and 0.1% TFA/CH₃CN as eluents.

To generate the des-octapeptide insulin (DOI), the dried single-chain DesDI precursor was treated with Trypsin-TPCK (10% w/w) in 1 M urea and 0.1 M ammonium bicarbonate for 24 h at room temperature. After completion of the cleavage as indicated by HPLC, the DOI was purified by preparative RP-HPLC on a C4 or C8 (20×250 mm, 5 μm, Higgins Analytical) column with 0.1% TFA/H₂O (A) and 0.1% TFA/CH₃CN (B) as elution buffers.

Semi-synthesis with Diol containing octapeptides. The DOI from the above step was combined with de protected octapeptide Gly-Phe-Phe-Tyr-Thr-Lys(DHBA)-Pro-Thr (reacted as a crude or previously HPLC-purified) in presence of 10% w/w Trypsin-TPCK (K. Inouye, K. Watanabe, K. Morihara, Y. Tochino, T. Kanaya, J. Emura, S. Sakakibara, J. Am. Chem. Soc. 1979, 101, 751-752). Generally, a 1:5 molecular ratio was used for trypsin-mediated ligation, typically 3-9 mg of DOI were dissolved along with similar 3-9 mg of octapeptide in 200 μl of a mixed solvent system containing Tris-acetate (pH 8.5), 1,4-butanediol and dimethylacetamide. pH was adjusted to 7.5 with 2 μl of 4-Methylmorpholine and the reaction was carried out for 24-48 h. Full-length insulin product proteins was purified by preparative RP-HPLC on a C8 column with 0.1% TFA/H₂O (A) and 0.1% TFA/CH₃CN (B) as elution buffers. Identity was confirmed by LC-MS. The DOI precursor and resultant GRI candidate compounds are given in the SEQ ID section. After semi-synthesis, reaction mixtures were purified by RP-HPLC as described above. The general disulfide conjugation method is as follows: GBE-1-C(NPYS)-OH, 1.5 equivalents were reacted with an SCI containing a free thiol group (2-4 mg/ml). The reaction are performed in a buffer consisting of ammonium bicarbonate (0.1 M), urea (1M) at pH 8.5 and at room temperature for 15-60 min. The reaction mixture was re-purified by RP-HPLC.

Cell culture. Human hepatocellular carcinoma cell line HepG2 was cultured in Dulbecco's Modified Eagle Medium (DMEM), supplemented with 10% fetal bovine serum (FBS), 1% penicillin/streptomycin as recommended by the American Type Culture Collection. A protocol employing 24-h serum starvation wherein FBS was applied at 70-75% confluence. After starvation, cells were treated in parallel with a set of insulin analogues in serum-free medium.

Real-time qPCR assays. Following serum starvation, HepG2 cells were treated with medium containing an insulin analogue (50 nM) for 8 h. In studies related to possible glucose responsiveness and lipid metabolism, the cells were treated with analogues for 3 or 4 h in media containing either low or normal glucose concentrations. Readouts were provided by downregulation of PEPCK and G6P and upregulation of ChREBP and SREBP. mRNA (messenger ribonucleic acid) abundances were measured in triplicate by quantitative polymerase chain reaction (qPCR). Samples were prepared as described by the vendor (One-Step rt-PCR reagent kits; Bio-Rad). The following sets of primers were used: (PEPCK), GTTCAATGCCAGGTTCCCAG (SEQ ID NO: 33) and TTGCAGGCCAGTTGTTGAC (SEQ ID NO: 34); (ChREBP), AGAGACAAGATCCGCCTGAA (SEQ ID NO: 35) and CTTCCAGTAGTTCCCTCCA (SEQ ID NO: 36); (SREBP), CGACATCGAAGACATGCTTCAG (SEQ ID NO: 37) and GGAAGGCTTCAAGAGAGGAGC (SEQ ID NO: 38); and (GAPDH), ATGGTTTACATGTTCCAATAT (SEQ ID NO: 39) and ATGAGGTCCACCACCCTGGTTG (SEQ ID NO: 40).

In-cell pIR immunoblotting. This cell-based assay probed insulin-dependent IR activation via fluorescent readouts. HepG2 cells were seeded (˜8000 cells/well) into a 96-well black plate with clear bottom and cultured (Fisher). After serum starvation in 100 μl plain Hanks' Balanced Salt Solution (HBSS) for 2 h at 37° C., serial analogue dilutions (100 μl) were applied to each well; cells were then incubated for 20 min at 37° C. After removing medium, 150 μl of 3.7% formaldehyde (Fisher) was added to each well, and plates incubated for 20 min at 37° C. 200 μl of 0.1% Triton-X-100 (Sigma) was then added to permeabilize the cells, followed by their fixation by 100-μl Odyssey Blocking Buffer (LI-COR). A blocking procedure was applied for 1 h at room temperature on an orbital shaker. Fixed cells were then exposed to the primary antibody (10 μL anti-pTyr 4G10 (Sigma) into 20 ml Blocking Buffer) overnight at 4° C. The secondary antibody (anti-mouse-IgG-800-CW antibody (Sigma) in 25 ml Blocking Buffer) was added after a wash. pTyr was detected via 800 nm emission. DRAQ5 (Fisher) was also applied to enable measurement of cell number via 700 nm emission. The fluorescence signals were detected on a LI-COR Infrared Imaging system (Odyssey) under settings as follows: offset 4 mm with setting “Intensity-Auto.”

Signaling assays in a mammalian cell line. HepG2 cell line (ATCC) were cultured in DMEM with 10% FBS, 1% penicillin/streptomycin per vendor's instruction. After 70-75% cell confluence, cells were serum-starved for 24 h and then exposed to medium containing an insulin analogue (each at a protein concentration of 50 nM). After lysis in RIPA buffer (containing protease- and phosphatase-inhibitor cocktails; Roche), the total protein concentrations in the lysate was determined by BCA assay (Thermo). Blotting protocols were modified from previous publication. Briefly, for p-IR/IR blotting, samples were probed by insulin receptor β (4B8) antibody (CST antibodies unless otherwise stated) or an equal mixture of anti-phospho-insulin receptor β (Tyr1150/1151); phospho-insulin receptor (Tyr1158) antibody (Thermo); phospho-insulin receptor (Tyr1334) antibody (Thermo); phospho-insulin receptor β (Tyr1345) monoclonal antiserum; and anti-phospho-insulin receptor (phospho-Tyr972) antibody (Abcam). Dilutions for these antibodies were 1:5000 in 5% bovine serum albumin. Antibodies for AKT blotting were p-AKT antibody (Ser473) (1:400) and AKT1/2/3 antiserum (H-136) (1:1000). For p-FOXO/FOXO and p-GSK-3/GSK-3 blotting, samples were probed by phospho-Fox01 (Thr24)/FoxO3a (Thr32) antibody, FOXO1 antibody, phospho-GSK-3α/β (Ser21/9), and GSK-3α/β antibody (purchased from CST; dilution 1:1000).

SEQUENCE ID SUMMARY
SEQ ID Compound Name Sequence/GBE and Diol Structures

SEQ ID NO: 1 (human proinsulin)
Phe-Val-Asn-Gln-His-Leu-Cys-Gly-Ser-His-Leu-Val-Glu-Ala-Leu-Tyr-Leu-
Val-Cys-Gly-Glu-Arg-Gly-Phe-Phe-Tyr-Thr-Pro-Lys-Thr-Arg-Arg-Glu-Ala-Glu-
Asp-Leu-Gln-Val-Gly-Gln-Val-Glu-Leu-Gly-Gly-Gly-Pro-Gly-Ala-Gly-Ser-Leu-
Gln-Pro-Leu-Ala-Leu-Glu-Gly-Ser-Leu-Gln-Lys-Arg-Gly-Ile-Val-Glu-Gln-Cys-Cys-
Thr-Ser-Ile-Cys-Ser-Leu-Tyr-Gln-Leu-Glu-Asn-Tyr-Cys-Asn
SEQ ID NO: 2 (human A chain)
Gly-Ile-Val-Glu-Gln-Cys-Cys-Thr-Ser-Ile-Cys-Ser-Leu-Tyr-Gln-Leu-Glu-
Asn-Tyr-Cys-Asn
SEQ ID NO: 3 (human B chain)
Phe-Val-Asn-Gln-His-Leu-Cys-Gly-Ser-His-Leu-Val-Glu-Ala-Leu-Tyr-Leu-Val-Cys-Gly-Glu-Arg-Gly-Phe-Phe-Tyr-Thr-Pro-
Lys-Thr
1 GlyA₋₁, Cys(GBE1-C)A₀, UA6,UA11-DOI (U = Sec; DOI = des-octapeptide[B23-B30]-insulin)

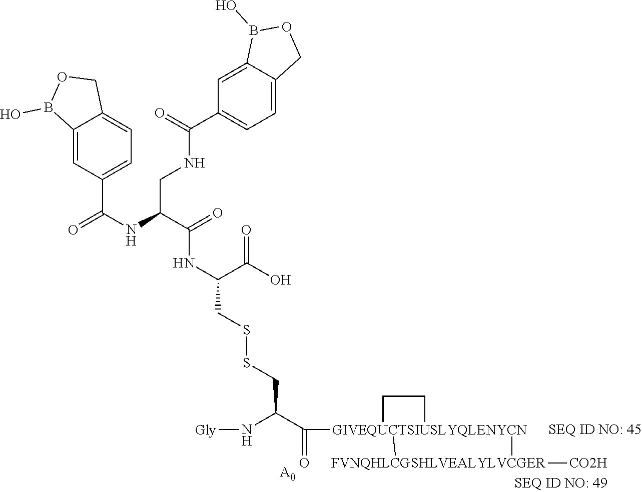

2. GlyA₋₁, Cys(GBE1-C)A₀, UA6, UA11, Lys(DHBA)B28 lispro (U = Sec)

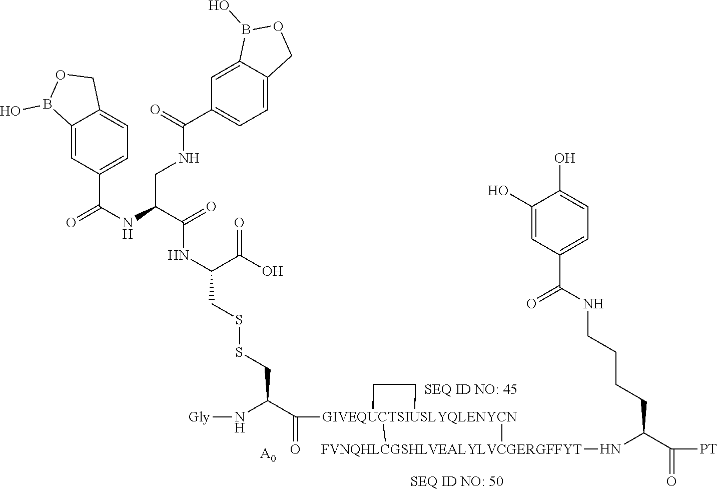

3 GlyA-1, Cys(GBE1-C)A0-DOI

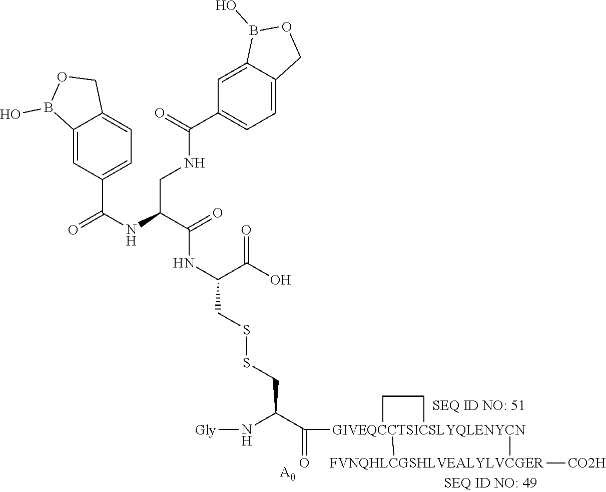

4 GlyA₋₁, Cys(GBE1-C)A₀, Lys(DHBA)B28-lispro

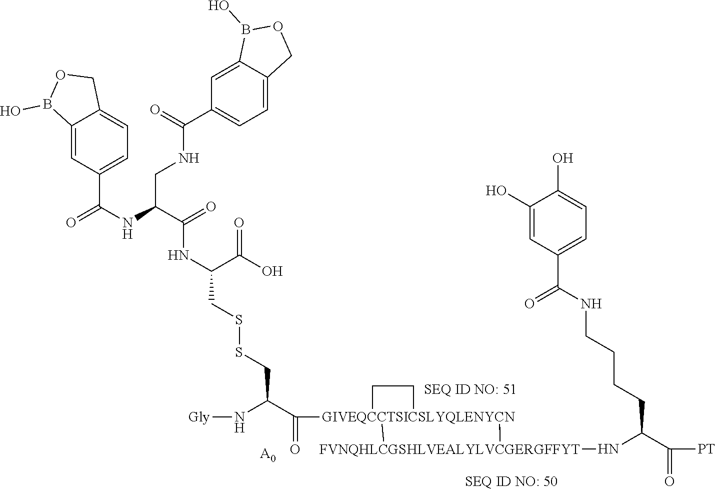

5 GlyA₋₁, D-Cys(GBE1-C)A₀-DOI

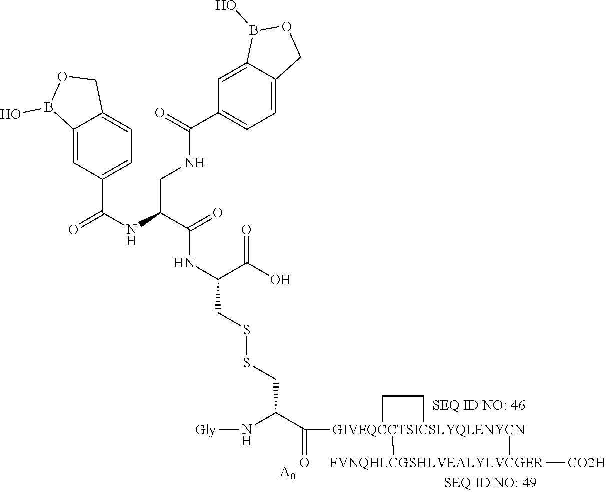

6 GlyA₋₁, D-Cys(GBE1-C)A₀, Lys(DHBA)B28-lispro

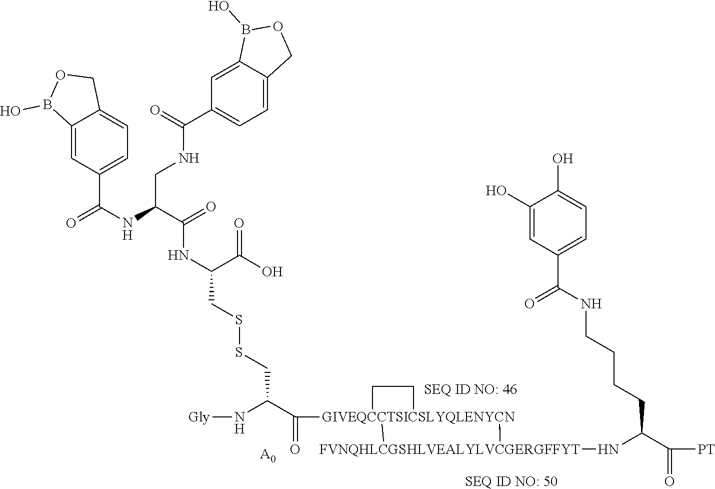

7 GlyA0, D-Cys(GBE1-C)A1, UA6,UA11-DOI

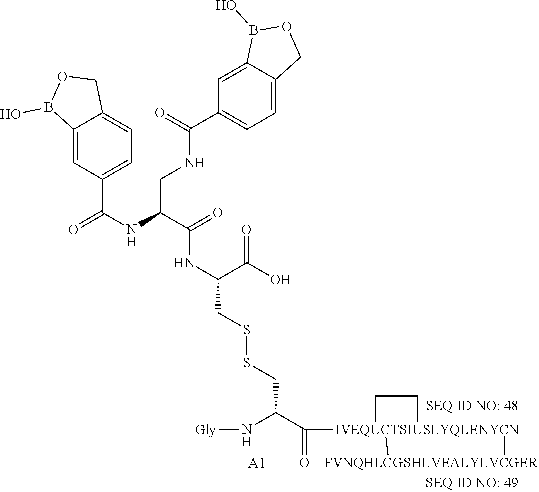

8 GlyA₀, D-Cys(GBE1-C)A1, UA6, UA11, Lys(DHBA)B28-lispro

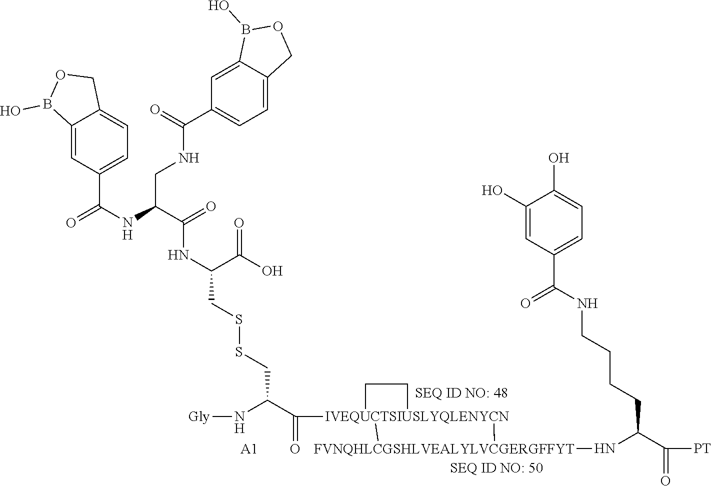

9 GlyA₀, D-Cys(GBE1-C)A1-DOI

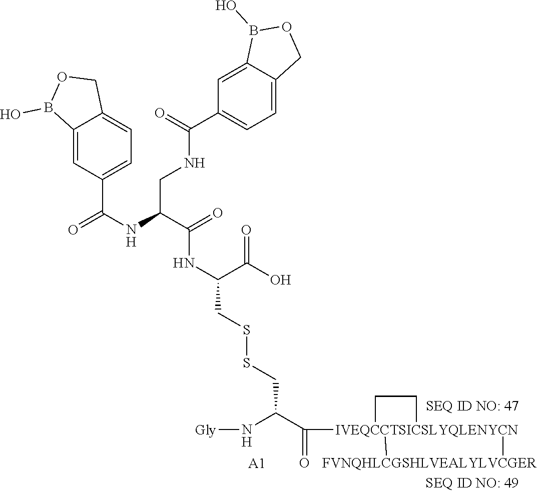

10 GlyA₀, D-Cys(GBE1-C)A1, Lys(DHBA)B28-lispro

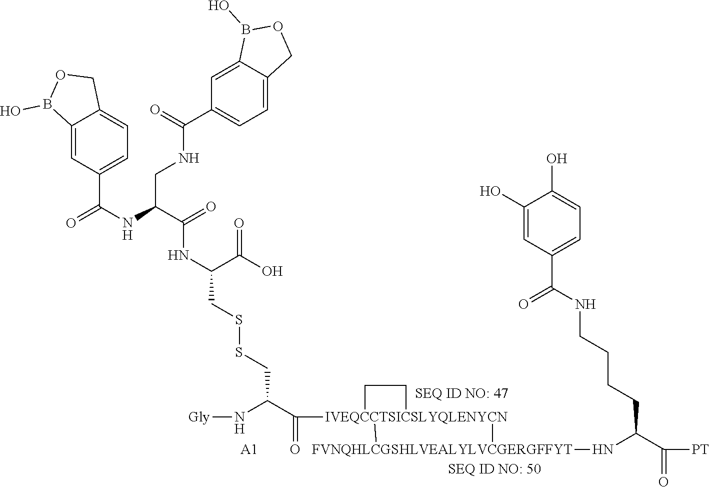

11 GlyA₀, Cys(GBE1-C)A1-DOI

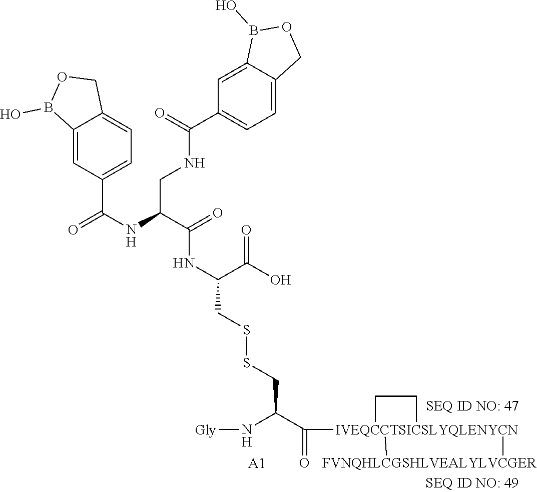

12 GlyA₀, Cys(GBE1-C)A1, Lys(DHBA)B28-lispro

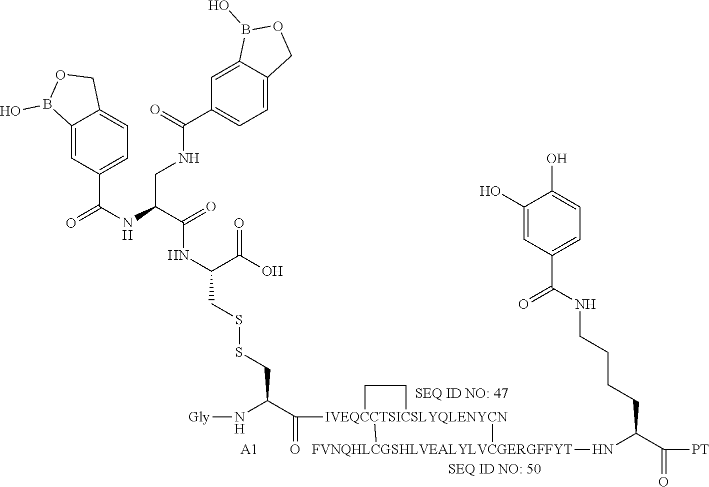

13 Cys(GBE1-C)A₀-DOI

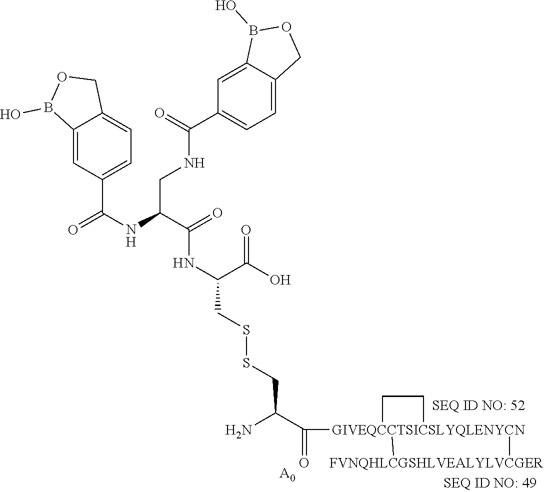

14. Cys(GBE1-C)A₀, Ly(DHBA)B28 lispro

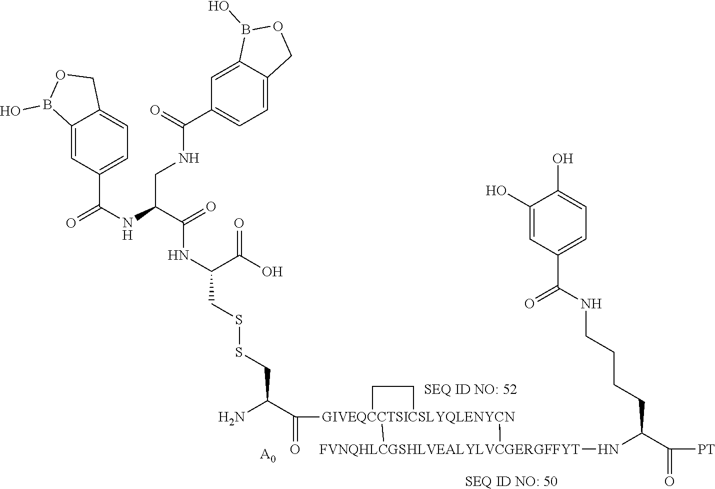

15 Cys(GBE1-C)A1-DOI

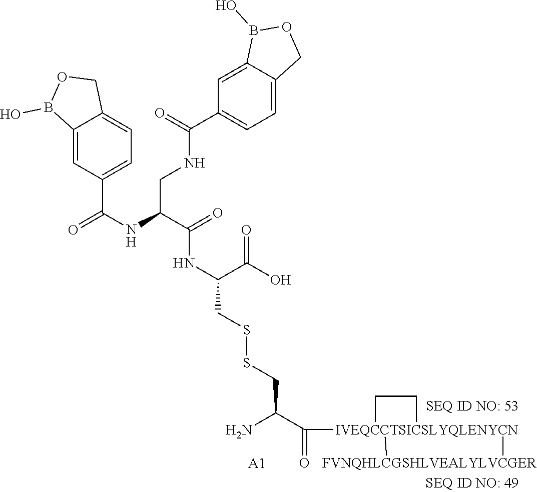

16 Cys(GBE1-C)A1, Lys(DHBA)B28-lispro

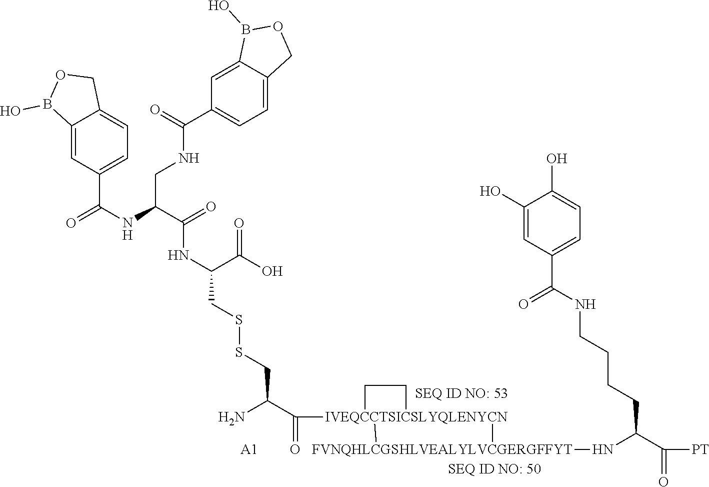

17 D-Cys(GBE1-C)A0-DOI

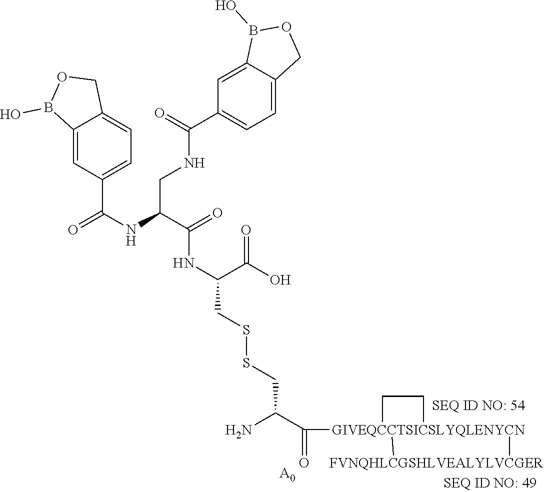

18 D-Cys(GBE1-C)A₀, Lys(DHBA)B28-lispro

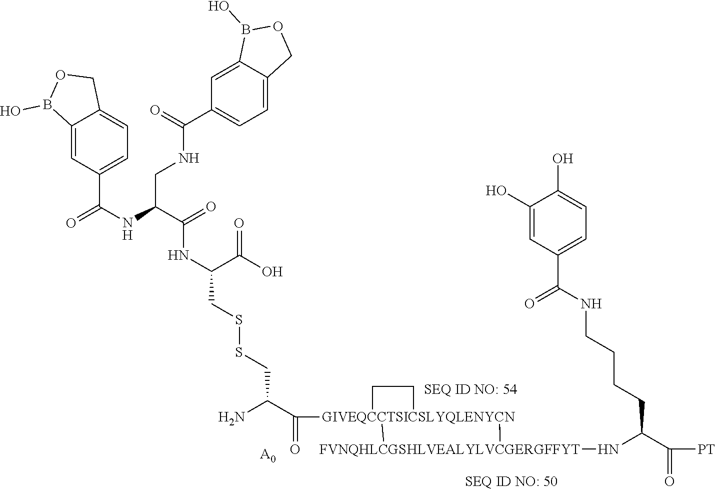

19 D-Cys(GBE1-C)A1-DOI

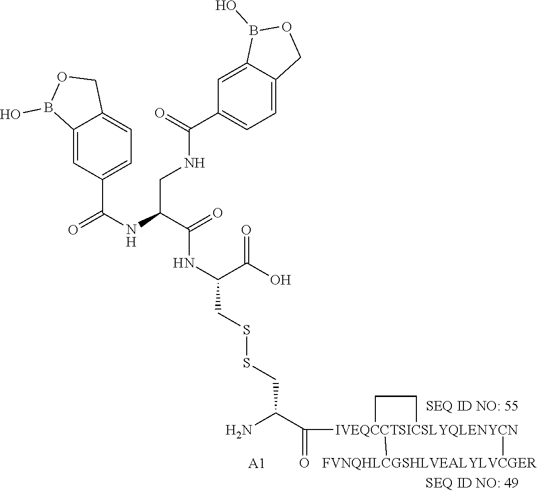

20. D-Cys(GBE1-C)A1, Lys(DHBA)B28-lispro

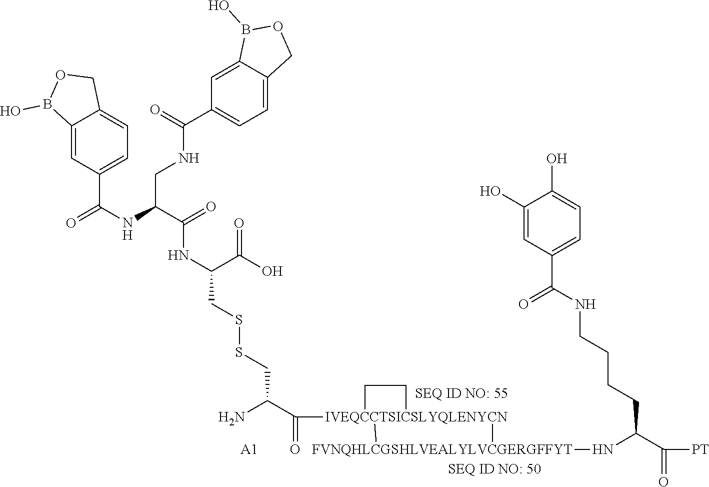

21 GlyA₋₁, Dap(GBE1)A₀-DOI

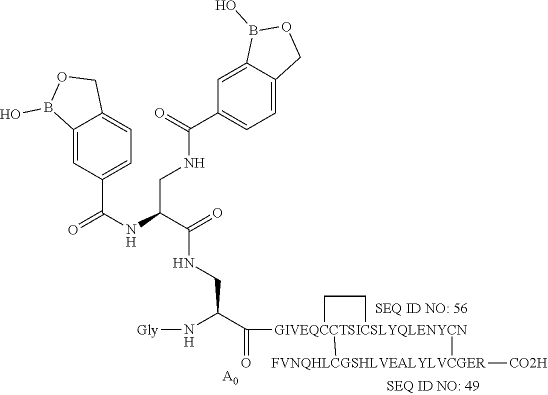

22 GlyA₋₁, Dap(GBE1)A₀, Lys(DHBA)B28-lispro

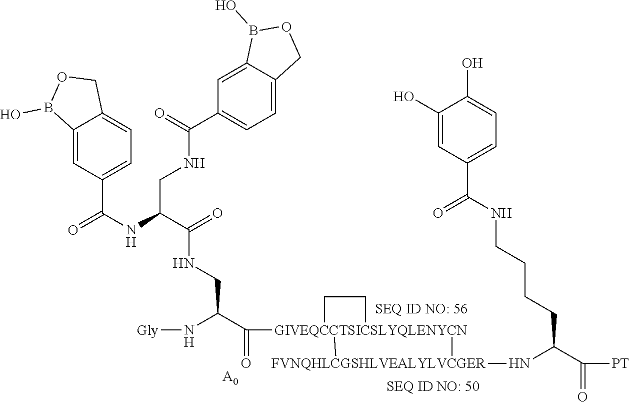

23 GlyA₋₁, Dap(GBE3)A₀-DOI

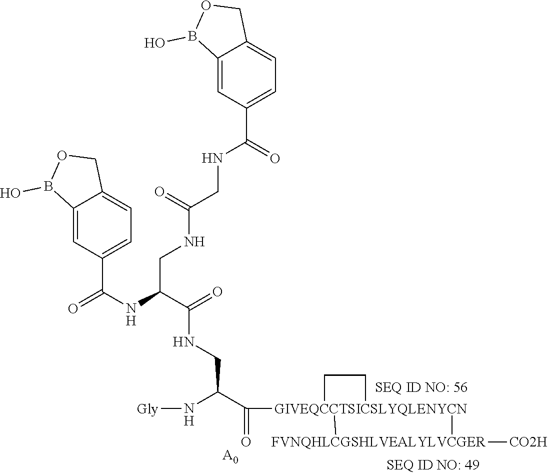

24 GlyA₋₁, Dap(GBE3)A₀, Lys(DHBA)B28-lispro

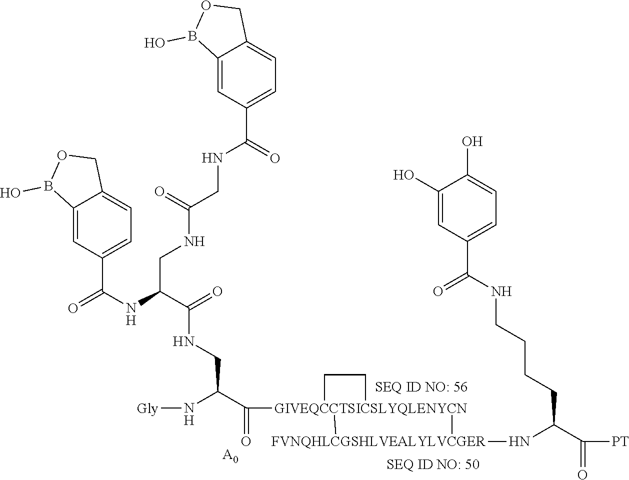

25 GlyA₀, D-Dap(GBE1)A1, HisA8-DOI

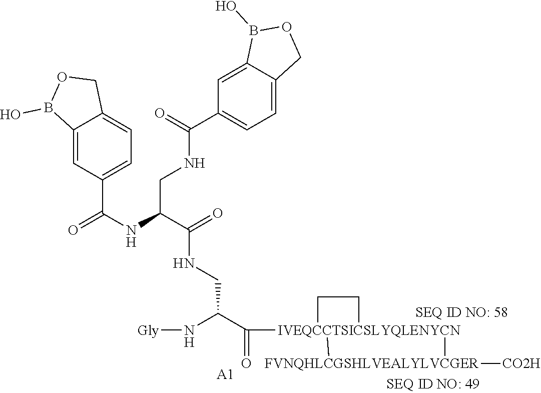

26 GlyA₀, D-Dap(GBE1)A1-DOI

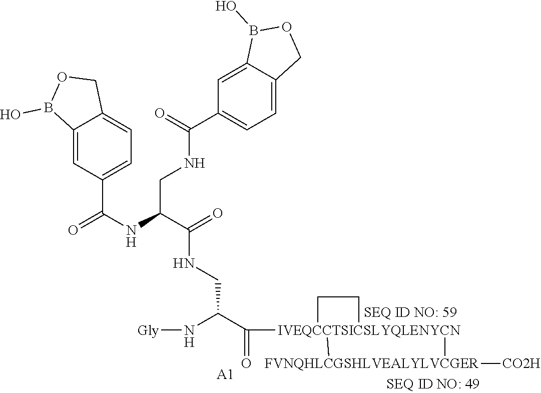

27 A₀, D-Dap(GBE1)A1, Lys(DHBA)B28-lispro

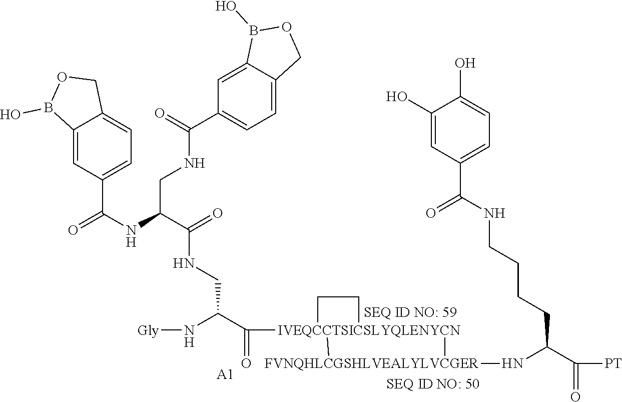

28 GlyA₀, D-Dap(GBE2)A1, HisA8-DOI

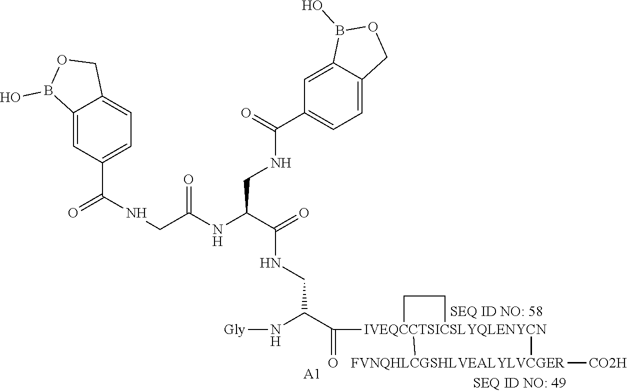

29 GlyA₀, D-Dap(GBE-3)A1, HisA8, Lys(DHBA)B28-lispro

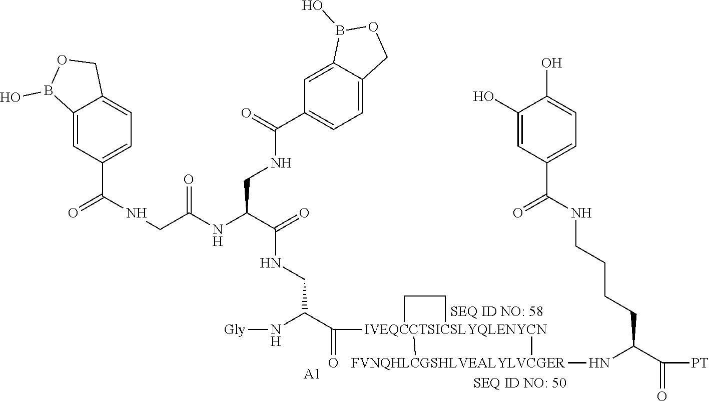

30 GlyA₀, D-Dab(GBE-3)A1, HisA8-DOI

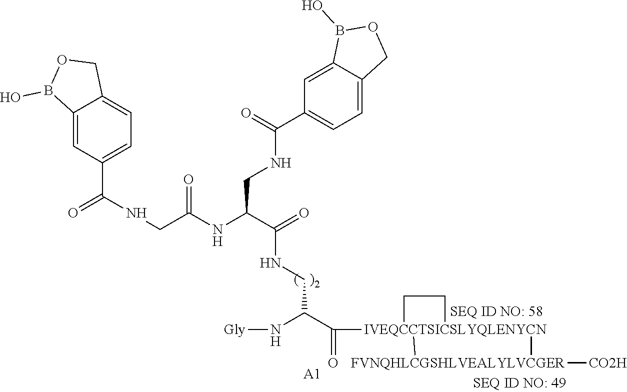

31 GlyA₀, D-Dab(GBE-3)A1, HisA8, Lys(DHBA)B28-lispro

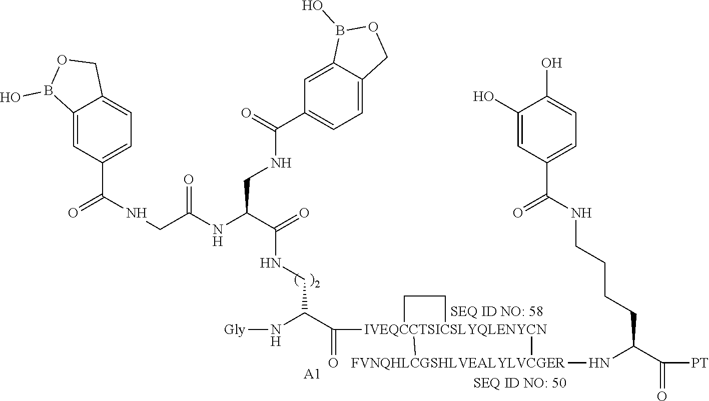

32 GlyA₀, D-Orn(GBE-3)A1, HisA8-DOI

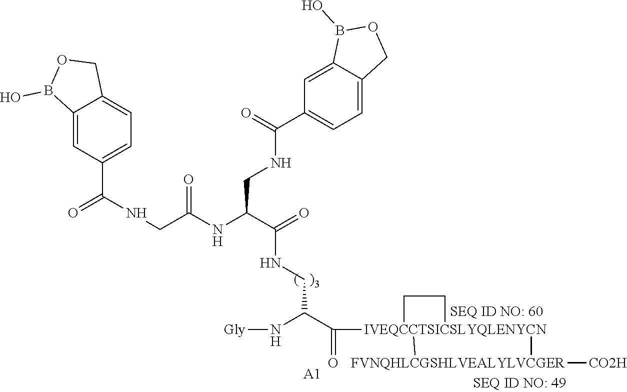

33 GlyA₀, D-Orn(GBE-3)A1, HisA8,K(DHBA)B28-lispro

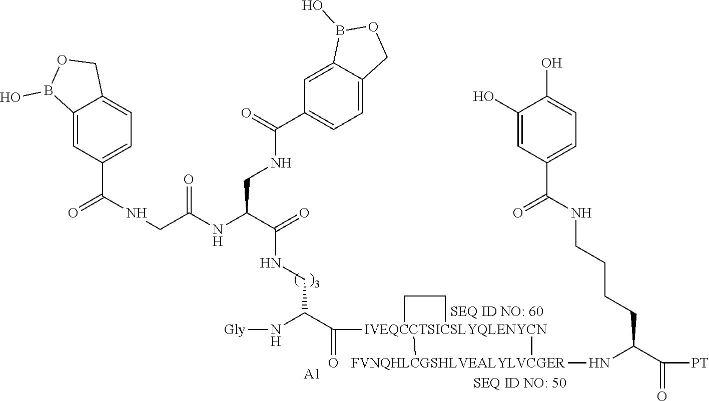

34 GlyA₀, D-Lys(GBE-3)A1, HisA8-DOI

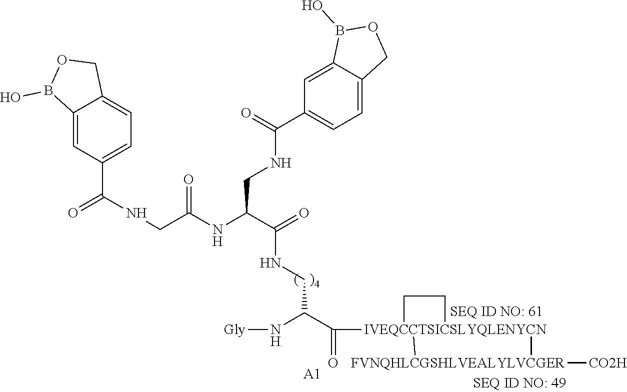

35 GlyA₀, D-Lys(GBE-3)A1, HisA8, Lys(DHBA)B28-lispro

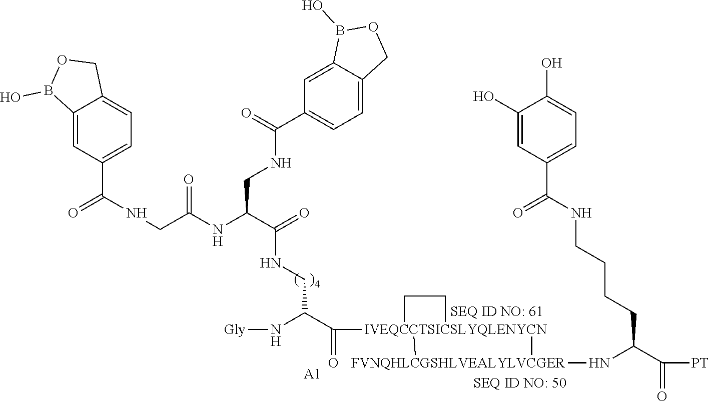

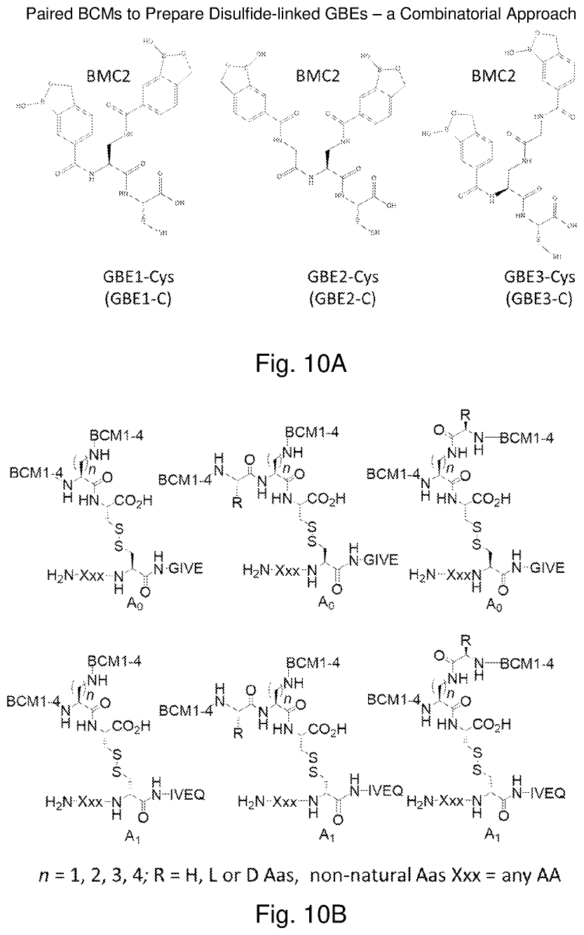

Note: U defines Selenocysteine Amino Acid
GBE-1 is defined in Fig. 10A-10B; GBE-2, GBE-3, GBE-4 are defined in Fig. 11A-11C.

NAMES AND SEQUENCES are summarized in Table 1 as follows. GRI-1, 2, 3, 4 and 5 are labeled in appropriate rows. “Single-chain sequences” and des-octapeptide[B23-B30]-insulin analogue fragments (“DOI”s) pertain to synthetic intermediates and not to GRIs of the present invention.

TABLE 1

| Seq | CysA₀ A-Chain | |
|---|---|---|
| ID | extension | Sequence |
| | GlyA₋₁, CysA₀-DesDi | FVNQHLCGSHLVEALYLVCGERGFFYTK--- |
| | single chain | GC(SH)GIVEQCCTSICSLYQLENYCN |
| | | SEQ ID NO: 84 |
| | GlyA₋₁, Cys(GBE1- | FVNQHLCGSHLVEALYLVCGERGFFYTK--- |
| | C)A₀-DesDi single | GC(GBE1-C)GIVEQCCTSICSLYQLENYCN |
| | chain | SEQ ID NO: 85 |
| | GlyA₋₁, Cys(GBE1- | FVNQHLCGSHLVEALYLVCGER (SEQ ID NO: |
| | C)A₀-DOI | 86)_GC(GBE1-C)GIVEQCCTSI |
| | | CSLYQLENYCN (SEQ ID NO: |
| | | 92) |
| | GlyA₋₁, Cys(GBE1- | FVNQHLCGSHLVEALYLVCGERGFFYTK(DH |
| | C)A₀, | BA)PT (SEQ ID NO: 89)_GC(GBE1- |
| | Lys(DHBA)B28-KP | C)GIVEQCCTSICSLYQLENYCN (SEQ ID NO: |
| | (“GRI-2”) | 92) |
| | Description: | |
| Seq | CysA₀ A-Chain | |
| ID | extension | Sequence |
| | GlyA₀, D-CysA1- | FVNQHLCGSHLVEALYLVCGERGFFYTK |
| | Desdi | (SEQ ID NO: 90)--- |
| | | GdC(SH)IVEQCCTSICSLYQLENYCN SEQ ID |
| | | NO: 110) |
| | GlyA₀, D-Cys(GBE1- | FVNQHLCGSHLVEALYLVCGERGFFYTK |
| | C)A1-DesDi single | (SEQ ID NO: 90)---GdC(GBE1- |
| | chain | C)IVEQCCTSICSLYQLENYCN (SEQ ID NO: |
| | | 93) |
| | GlyA₀, D-Cys(GBE1- | FVNQHLCGSHLVEALYLVCGER (SEQ ID NO: |
| | C)A1-DOI | 86)_GdC(GBE1-C)IVEQCCTSICSLYQLENYCN |
| | | (SEQ ID NO: 93) |
| | GlyA₀, D-Cys(GBE1- | FVNQHLCGSHLVEALYLVCGERGFFYTK(DH |
| | C)A1, | BA)PT (SEQ ID NO: 89)_GdC(GBE1- |
| | Lys(DHBA)B28- | C)IVEQCCTSICSLYQLENYCN (SEQ ID NO: |
| | lispro (“GRI-1”) | 93) |
| Seq | Description: CysA₀ | |
| ID | with Seleno-Cys | Sequence |
| | GlyA₋₁, CysA₀, | FVNQHLCGSHLVEALYLVCGERGFFYTK |
| | SecA6, SecA11- | (SEQ ID NO: 90)--- |
| | DesDi single chain | GC(SH)GIVEQ[Sec]CTSI[Sec]SLYQLENYCN |
| | | (SEQ ID NO: 111) |
| | GlyA₋₁, Cys(GBE1- | FVNQHLCGSHLVEALYLVCGERGFFYTK |
| | C)A₀, SecA6, | (SEQ ID NO: 90)---GC(GBE1-C) |
| | SecA11-DesDi single | GIVEQ[Sec]CTSI[Sec]SLYQLENYCN (SEQ |
| | chain | ID NO: 94) |
| | GlyA₋₁, Cys(GBE1- | FVNQHLCGSHLVEALYLVCGER (SEQ ID NO: |
| | C)A₀, SecA6, | 86)_GC(GBE1-C) |
| | SecA11-DOI | GIVEQ[Sec]CTSI[Sec]SLYQLENYCN (SEQ |
| | | ID NO: 94) |
| | GlyA₋₁, Cys(GBE1- | FVNQHLCGSHLVEALYLVCGERGFFYTK(DH |
| | C)A₀, SecA6, | BA)PT (SEQ ID NO: 89)_GC(GBE1-C) |
| | SecA11, | GIVEQ[Sec]CTSI[Sec]SLYQLENYCN (SEQ |
| | Lys(DHBA)B28- | ID NO: 94) |
| | lispro | |
| | (“GRI-4”) | |
| | Description: D- | |
| Seq | CysA1 with Seleno- | |
| ID | Cys | Sequence |
| | GlyA₀, D-CysA1, | FVNQHLCGSHLVEALYLVCGERGFFYTK |
| | SecA6, SecA11- | (SEQ ID NO: 90)--- |
| | DesDi single chain | G[dC]IVEQ[Sec]CTSI[Sec]SLYQLENYCN (SEQ |
| | | ID NO: 112) |
| | GlyA₀, D-Cys(GBE- | FVNQHLCGSHLVEALYLVCGERGFFYTK |
| | 1-C)A1, SecA6, | (SEQ ID NO: 90)---G[dC(GBE1-C)] |
| | SecA11-DesDi | IVEQ[Sec]CTSI[Sec]SLYQLENYCN (SEQ ID |
| | | NO: 95) |
| | GlyA₀, D-Cys(GBE1- | FVNQHLCGSHLVEALYLVCGER (SEQ ID NO: |
| | C)A1, SecA6, | 86)_G[dC(GBE1-C)] |
| | SecA11-DOI | IVEQ[Sec]CTSI[Sec]SLYQLENYCN (SEQ ID |
| | | NO: 95) |
| | GlyA₀, D-Cys | FVNQHLCGSHLVEALYLVCGERGFFYTK(DH |
| | (GBE1-C)A1, SecA6, | BA)PT (SEQ ID NO: 89)_G[dC(GBE1- |
| | SecA11, | C)]IVEQ[Sec]CTSI[Sec]SLYQLENYCN |
| | Lys(DHBA)B28- | (SEQ ID NO: 95) |
| | lispro | |
| | (“GRI-3”) | |
| | GlyA₀, D-Cys(GBE1- | FVNQHLCGSHLVEALYLVCGERGFF[Sar][ADP]I |
| | C)A1, SecA6, | (SEQ ID NO: 72),_G[dC(GBE-1)] |
| | SecA11, | IVEQ[Sec]CTSI[Sec]SLYQLENYCN |
| | Lys(DHBA)B28- | (SEQ ID NO: 95) |
| | lispro | |
| Seq | Backbone Dap | |
| ID | modification | Sequence |
| | Cys(GBE-1)A₀-DOI | FVNQHLCGSHLVEALYLVCGER (SEQ ID NO: |
| | | 86)---C(GBE-1)GIVEQCCTSICSLYQLENYCN |
| | | (SEQ ID NO: 96) |
| | GlyA₀, D-Cys(GBE- | FVNQHLCGSHLVEALYLVCGER (SEQ ID NO: |
| | 1)A1, HisA8-DOI | 86)---GdC(GBE-1)IVEQCCHSICSLYQLENYCN |
| | | (SEQ ID NO: 97) |
| | Backbone A1-D-Dap | |
| Seq | on resin | |
| ID | modifications | Sequence |
| | Boc-B1, GlyA₀, D- | Boc-FVNQHLCGSHLVEALYLVCGERGFFYTK |
| | Dap(Mtt)A1, HisA8- | (SEQ ID NO: 90)--- |
| | DesDi-R | G[dDap(Mtt)]IVEQCCHSICSLYQLENYCN |
| | | (SEQ ID NO: 98) |
| | GlyA₀, D-Dap(Fmoc- | Boc-FVNQHLCGSHLVEALYLVCGERGFFYTK |
| | Dap(Fmoc))A1, | (SEQ ID NO: 90)---G[dDap(Fmoc- |
| | HisA8-DesDi-R | Dap(Fmoc))]IVEQCCHSICSLYQLENYCN (SEQ |
| | | ID NO: 99) |
| | GlyA₀, D-Dap(H2N- | Boc-FVNQHLCGSHLVEALYLVCGERGFFYTK |
| | Dap(NH2))A1, HisA8 | (SEQ ID NO: 90)---G[dDap(H2N- |
| | -DesDi-R | Dap(NH2))]IVEQCCHSICSLYQLENYCN (SEQ |
| | | ID NO: 100) |
| | GlyA₀, D- | Boc-FVNQHLCGSHLVEALYLVCGERGFFYTK |
| | Dap(BCM2- | (SEQ ID NO: 90)---G[dDap(GBE- |
| | Dap(BCM2))A1, | Dap(GBE))]IVEQCCHSICSLYQLENYCN (SEQ |
| | HA8-Desdi-R | ID NO: 101) |
| | GlyA₀, D- | FVNQHLCGSHLVEALYLVCGERGFFYTK |
| | Dap(GBE2))A1, | (SEQ ID NO: 90)---G[dDap(GBE- |
| | HisA8-DesDi | Dap(GBE))]IVEQCCHSICSLYQLENYCN (SEQ |
| | | ID NO: 101) |
| | Backbone A1-D-Dap | |
| Seq | on resin | |
| ID | modifications | Sequence |
| | Boc-B1, GlyA₀, D- | Boc-FVNQHLCGSHLVEALYLVCGERGFFYTK |
| | Dap(Mtt)A1, HisA8- | (SEQ ID NO: 90)--- |
| | Desdi-R | G[dDap(Mtt)]IVEQCCHSICSLYQLENYCN |
| | | (SEQ ID NO: 98) |
| | Boc-B1, GlyA₀, D- | Boc-FVNQHLCGSHLVEALYLVCGERGFFYTK |
| | Dap(NH2)A1, HisA8 | (SEQ ID NO: 90)--- |
| | -Desdi-R | G[dDap(NH2)]IVEQCCHSICSLYQLENYCN |
| | | SEQ ID NO: 102) |
| | Boc-B1, GlyA₀, D- | Boc-FVNQHLCGSHLVEALYLVCGERGFFYTK |
| | Dap(Fmoc- | (SEQ ID NO: 90)---G[dDap(Fmoc- |
| | Dap(Mtt))A1, HA8- | Dap(Mtt)]IVEQCCHSICSLYQLENYCN (SEQ ID |
| | Desdi-R | NO: 103) |
| | Boc-B1, GlyA₀, D- | Boc-FVNQHLCGSHLVEALYLVCGERGFFYTK |
| | Dap(H2N- | (SEQ ID NO: 90)---G[dDap(H2N- |
| | Dap(Mtt))A1, HA8- | Dap(Mtt)]IVEQCCHSICSLYQLENYCN (SEQ |
| | Desdi-R | ID NO: 104) |
| | Boc-B1, GA₀, D- | Boc-FVNQHLCGSHLVEALYLVCGERGFFYTK |
| | Dap(Gly- | (SEQ ID NO: 90)---G[dDap(Gly- |
| | Dap(NH2))A1, | Dap(NH2)]IVEQCCHSICSLYQLENYCN (SEQ |
| | HisA8-DesDi-R | ID NO: 105) |
| | GlyA₀, D-Dap(H2N- | FVNQHLCGSHLVEALYLVCGERGFFYTK |
| | Dap(FmocG))A1, | (SEQ ID NO: 90)---G[dDap(H2N-Dap(Fmoc- |
| | HisA8-DesDi | Gly)]IVEQCCHSICSLYQLENYCN (SEQ ID NO: |
| | | 106) |
| | GlyA₀, D- | Boc-FVNQHLCGSHLVEALYLVCGERGFFYTK |
| | Dap(BCM2-Gly- | (SEQ ID NO: 90)---G[dDap(BCM2-Gly- |
| | Dap(BCM2))A1, | Dap(BCM2))]IVEQCCHSICSLYQLENYCN |
| | HisA8-DesDi | (SEQ ID NO: 107) |
| | GlyA₀, D-Dap(GBE- | Boc-FVNQHLCGSHLVEALYLVCGER (SEQ ID |
| | Gly-Dap(GBE))A1, | NO: 86)---G[dDap(BCM2- |
| | HisA8-DOI | Dap(BCM2))]IVEQCCHSICSLYQLENYCN |
| | | (SEQ ID NO: 108) |
| | Boc-GlyA₋₁, | Boc-FVNQHLCGSHLVEALYLVCGERGFFYTK |
| | Dap(alloc)A₀, HisA8- | (SEQ ID NO: 90)--- |
| | DesDi | G[Dap(Alloc)]GIVEQCCHSICSLYQLENYCN |
| | | (SEQ ID NO: 109) |
| | Boc-GlyA₀, D- | Boc-FVNQHLCGSHLVEALYLVCGERGFFYTK |
| | Dap(Mtt)A1, HisA8- | (SEQ ID NO: 90)--- |
| | DesDi | G[dDap(Mtt)]IVEQCCHSICSLYQLENYCN |
| | | (SEQ ID NO: 98) |
| | Boc-GlyA₋₁, | Boc-FVNQHLCGSHLVEALYLVCGERGFFYTK |
| | Dap(alloc)A₀-DesDi | (SEQ ID NO: 90)--- |
| | | G[Dap(Alloc)]GIVEQCCTSICSLYQLENYCN |
| | | (SEQ ID NO: 141) |
| | Boc-GlyA₀, D- | Boc-FVNQHLCGSHLVEALYLVCGERGFFYTK |
| | Dap(alloc)A1-DesDi | (SEQ ID NO: 90)--- |
| | | G[dDap(alloc)]IVEQCCTSICSLYQLENYCN |
| | | (SEQ ID NO: 141) |
| | Desdi Intermediates | |
| Seq | for thiol-based | |
| ID | linkages | Sequence |
| | Cys(Acm)A₀, HisA8 | FVNQHLCGSHLVEALYLVCGERGFFYTK |
| | -DesDi | (SEQ ID NO: 90)--- |
| | | C(Acm)GIVEQCCHSICSLYQLENYCN (SEQ ID |
| | | NO: 113) |
| | Cys(Acm)A₀, HisA8- | FVNQHLCGSHLVEALYLVCGERGFFYTK |
| | DesDi-TCI | (SEQ ID NO: 90) |
| | | C(Acm)GIVEQCCHSICSLYQLENYCN (SEQ |
| | | ID NO: 113) |
| | Cys(Acm)A₀, HisA8- | FVNQHLCGSHLVEALYLVCGER (SEQ ID NO: |
| | DOI | 86)_C(Acm)GIVEQCCHSICSLYQLENYCN |
| | | (SEQ ID NO: 113) |
| | GlyA₋₁, | FVNQHLCGSHLVEALYLVCGERGFFYTK |
| | Cys(Npys))A₀, | (SEQ ID NO: 90)-- |
| | SecA6, SecA11- | GC(Npys)GIVEQ[Sec]CTSI[Sec]SLYQLENYCN |
| | DesDi | SEQ ID NO: 114) |
| | GlyA₋₁, Cys(Cys)A₀, | FVNQHLCGSHLVEALYLVCGERGFFYTK |
| | SecA6, SecA11- | (SEQ ID NO: 90)--- |
| | DesDi | GC(Cys)GIVEQ[Sec]CTSI[Sec]SLYQLENYCN |
| | | (SEQ ID NO: 115) |
| | GlyA₀, D- | FVNQHLCGSHLVEALYLVCGERGFFYTK |
| | Cys(Npys)A1, | (SEQ ID NO: 90)--- |
| | SecA6, SecA11- | G[dC(Npys)]IVEQ[Sec]CTSI[Sec]SLYQLENYCN |
| | DesDi | (SEQ ID NO: 116) |
| | GlyA₀, D- | FVNQHLCGSHLVEALYLVCGERGFFYTK |
| | Cys(Cys)A1, SecA6, | (SEQ ID NO: 90)--- |
| | SecA11-DesDi | G[dC(Cys)]IVEQ[Sec]CTSI[Sec]SLYQLENYCN |
| | | (SEQ ID NO: 117) |
| | GlyA₀, D- | FVNQHLCGSHLVEALYLVCGERGFFYTK |
| | Cys(Acm)A1, HisA8- | (SEQ ID NO: 90)---G[d- |
| | DesDi | C(Acm)]IVEQCCHSICSLYQLENYCN (SEQ ID |
| | | NO: 118) |
| | GlyA₀, D- | FVNQHLCGSHLVEALYLVCGER (SEQ ID NO: |
| | Cys(Acm)A1, HisA8- | 86)_G[dC(Acm)]IVEQCCHSICSLYQLENYCN |
| | DOI | (SEQ ID NO: 118) |
| | Initial A1 and A0 | |
| | GRI Prototypes | Sequence |
| | GlyA₋₁, Cys(GBE1- | FVNQHLCGSHLVEALYLVCGERGFFYTK(DH |
| | C)A₀, | BA)PT(SEQ ID NO: 89)_GC(GBE1- |
| | Lys(DHBA)B28- | C)GIVEQCCTSICSLYQLENYCN (SEQ ID NO: |
| | lispro “GRI-2” | 92) |
| | GlyA₀, D-Cys(GBE1- | FVNQHLCGSHLVEALYLVCGERGFFYTK(DH |
| | C)A1, | BA)PT (SEQ ID NO: 89)_GdC(GBE1- |
| | Lys(DHBA)B28- | C)IVEQCCTSICSLYQLENYCN (SEQ ID NO: |
| | lispro “GRI-1” | 93) |
| | GlyA₋₁, Cys(GBE1- | FVNQHLCGSHLVEALYLVCGERGFFYTK(DH |
| | C)A₀, Sec6, SecA11, | BA)PT (SEQ ID NO: 89)_GC(GBE1- |
| | Lys(DHBA)B28- | C)GIVEQUCTSIUSLYQLENYCN |
| | lispro | (SEQ ID NO: 94) |
| | “GRI-4” | |
| | GlyA₀, D-Cys(GBE1- | FVNQHLCGSHLVEALYLVCGERGFFYTK(DH |
| | C)A1, | BA)PT (SEQ ID NO: 89)_GdC(GBE1- |
| | Lys(DHBA)B28- | C)IVEQUCTSIUSLYQLENYCN (SEQ ID NO: |
| | lispro | 119) |
| | “GRI-3” | |
| | GRI-1 and Controls | Sequence |
| | GlyA₀, D-Cys(GBE1- | FVNQHLCGSHLVEALYLVCGERGFFYTK(DH |
| | C)A1, | BA)PT (SEQ ID NO: 89)_GdC(GBE1- |
| | Lys(DHBA)B28- | C)IVEQCCTSICSLYQLENYCN (SEQ ID NO: |
| | lispro | 93) |
| | GlyA₀, D-Cys(GBE1- | FVNQHLCGSHLVEALYLVCGERGFFYTKPT |
| | C)A1-lispro | (SEQ ID NO: 88) G[dC(DAZ4- |
| | | C)]IVEQCCTSICSLYQLENYCN (SEQ ID NO: |
| | | 119) |
| | Cys(Acm)A₀, HisA8, | FVNQHLCGSHLVEALYLVCGERGFFYTK(DH |
| | Lys(DHBA)B28- | BA)PT (SEQ ID NO: 89)_ |
| | lispro | C(Acm)GIVEQCCHSICSLYQLENYCN (SEQ |
| | | ID NO: 113) |
| | GlyA₀, D- | FVNQHLCGSHLVEALYLVCGERGFFYTK(DH |
| | Cys(Cys)A1, | BA)PT (SEQ ID NO: 89) |
| | Lys(DHBA)B28- | G[dC(Cys)]IVEQCCTSICSLYQLENYCN (SEQ |
| | lispro | ID NO: 93) |
| | A-Chain GBE, | |
| | (Diol)B28 Analogues | Sequence |
| | GlyA₋₁, Cys(GBE1- | FVNQHLCGSHLVEALYLVCGERGFFYTO(DH |
| | C)A₀, | BA)PT (SEQ ID NO: 91) GC(GBE1- |
| | Orn(DHBA)B28, | C)GIVEQCCTSICSLYQLENYCN (SEQ ID NO: |
| | ProB29 | 92) |
| | (“GRI-5”) | |
| | GlyA₀, D-Cys(GBE1- | FVNQHLCGSHLVEALYLVCGERGFFYTO(DH |
| | C)A1, | BA)PT (SEQ ID NO: 91) GdC(GBE1- |
| | Orn(DHBA)B28, | C)IVEQCCTSICSLYQLENYCN (SEQ ID NO: |
| | ProB29 | 93) |
| | GlyA₋₁, | FVNQHLCGSHLVEALYLVCGERGFFYTO(DH |
| | Dap(GBE1)A₀, | BA)PT (SEQ ID NO: 91) |
| | Orn(DHBA)B28, | GDap(GBE1)GIVEQCCTSICSLYQLENYCN |
| | ProB29 | (SEQ ID NO: 120) |
| | GlyA₋₁, | FVNQHLCGSHLVEALYLVCGERGFFYTK(DH |
| | Dap(GBE2)A₀, | BA)PT (SEQ ID NO: 89) |
| | Lys(DHBA)B28- | GDap(GBE2)GIVEQCCTSICSLYQLENYCN |
| | lispro | (SEQ ID NO: 120) |
| | GlyA₋₁, | FVNQHLCGSHLVEALYLVCGERGFFYTO(DH |
| | Dap(GBE2)A₀, | BA)PT (SEQ ID NO: 91) |
| | Orn(DHBA)B28, | GDap(GBE2)GIVEQCCTSICSLYQLENYCN |
| | ProB29 | (SEQ ID NO: 120) |
| | GlyA₋₁, | FVNQHLCGSHLVEALYLVCGERGFFYTK(DH |
| | Dap(GBE4)A₀, | BA)PT (SEQ ID NO: 89) |
| | Ly(DHBA)B28- | GDap(GBE4]GIVEQCCTSICSLYQLENYCN |
| | lispro | (SEQ ID NO: 120) |
| | GlyA₋₁, | FVNQHLCGSHLVEALYLVCGERGFFYTO(DH |
| | Dap(GBE4)A₀, | BA)PT (SEQ ID NO: 91) |
| | O(DHBA)B28-KP | GDap(GBE4)GIVEQCCTSICSLYQLENYCN |
| | | (SEQ ID NO: 120) |
| | GlyA₋₁, | FVNQHLCGSHLVEALYLVCGERGFFYTO(DH |
| | Dap(GBE5)A₀, | BA)PT (SEQ ID NO: 91) |
| | O(DHBA)B28, | GDap(GBE5)GIVEQCCTSICSLYQLENYCN |
| | ProB29 | (SEQ ID NO: 120) |
| | GlyA₋₁, | FVNQHLCGSHLVEALYLVCGERGFFYTK(DH |
| | Dap(GBE5)A₀, | BA)PT (SEQ ID NO: 89) |
| | Lys(DHBA)B28- | GDap(GBE5)GIVEQCCTSICSLYQLENYCN |
| | lispro | (SEQ ID NO: 93) |
| | GlyA₀, D- | FVNQHLCGSHLVEALYLVCGERGFFYTK(DH |
| | Dap(GBE1)A1, | BA)PT (SEQ ID NO: 89) |
| | Lys(DHBA)B28- | GdDap(GBE1]IVEQCCTSICSLYQLENYCN |
| | lispro | (SEQ ID NO: 120) |
| | GlyA₀, D- | FVNQHLCGSHLVEALYLVCGERGFFYTO(DH |
| | Dap(GBE1)A1, | BA)PT (SEQ ID NO: 91) |
| | Orn(DHBA)B28, | GdDap(GBE1]IVEQCCTSICSLYQLENYCN |
| | ProB29 | (SEQ ID NO: 120) |
| | GlyA₀, D- | FVNQHLCGSHLVEALYLVCGERGFFYTK(DH |
| | Dap(GBE2)A1, | BA)PT (SEQ ID NO: 89) |
| | Lys(DHBA)B28- | GdDap(GBE2]IVEQCCTSICSLYQLENYCN |
| | lispro | (SEQ ID NO: 120) |
| | GlyA₀, D- | FVNQHLCGSHLVEALYLVCGERGFFYTO(DH |
| | Dap(GBE2)A1, | BA)PT (SEQ ID NO: 91) |
| | OrnB28(HBA), | GdDap(GBE2]IVEQCCTSICSLYQLENYCN |
| | ProB29 | (SEQ ID NO: 120) |
| | GlyA₀, D- | FVNQHLCGSHLVEALYLVCGERGFFYTK(DH |
| | Orn(GBE2)A1, | BA)PT (SEQ ID NO: 89) |
| | Lys(DHBA)B28- | GdO(GBE2)IVEQCCTSICSLYQLENYCN (SEQ |
| | lispro | ID NO: 122) |
| | GlyA₋₁, | FVNQHLCGSHLVEALYLVCGERGFFYTK(DH |
| | Orn(GBE3)A₀, | BA)PT (SEQ ID NO: 89) |
| | Lys(DHBA)B28- | GO(GBE3)GIVEQCCTSICSLYQLENYCN (SEQ |
| | lispro | ID NO: 123) |
| | GlyA₋₁, | FVNQHLCGSHLVEALYLVCGERGFFYG[APD] |
| | Dap(GBE2)A1, | G[APD] (SEQ ID NO: 126) GdC(GBE1- |
| | Gly[APD]B27G[APD] | C)IVEQCCTSICSLYQLENYCN (SEQ ID NO: |
| | B28 | 93) |
| | GlyA₀, D- | FVNQHLCGSHLVEALYLVCGERGFFYG[APD] |
| | Dap(GBE2)A1, | G[APD] (SEQ ID NO: 126) |
| | Gly[APD]B27, | GdDap(GBE2]IVEQCCTSICSLYQLENYCN |
| | Gly[APD]B28 | (SEQ ID NO: 125) |
| | A-Chain DHBA, | |
| | (GBE)B28 | |
| | Analogues | Sequence |
| | G_{A0}, D- | FVNQHLCGSHLVEALYLVCGERGFFYTK(GB |
| | Dap(DHBA)A1, | E1-CAc)PT (SEQ ID NO: 127) |
| | K(GBE1-CAc)B28- | GdDap(DHBA)IVEQCCTSICSLYQLENYCN |
| | lispro | (SEQ ID NO: 129) |
| | GlyA₋₁, | FVNQHLCGSHLVEALYLVCGERGFFYTK(GB |
| | Dap(DHBA)A0, | E1-CAc)PT (SEQ ID NO: 127) |
| | Lys(GBE1- | GdDap(DHBA)GIVEQCCTSICSLYQLENYCN |
| | CAc)B28-lispro | (SEQ ID NO: 130) |
| | GlyA₋₁, | FVNQHLCGSHLVEALYLVCGERGFFYTO(GB |
| | Dap(DHBA)A₀, | E6)PT (SEQ ID NO: 128) |
| | Orn(GBE6)B28, | GdDap(DHBA)GIVEQCCTSICSLYQLENYCN |
| | ProB29 | (SEQ ID NO: 130) |
| | GlyA₀, D- | FVNQHLCGSHLVEALYLVCGERGFFYTO(GB |
| | Dap(DHBA)A1, | E6)PT (SEQ ID NO: 128) |
| | Orn(GBE6)B28, | G[dDap(DHBA)]IVEQCCTSICSLYQLENYCN |
| | ProB29 | (SEQ ID NO: 129) |
| | GlyA₋₁, | FVNQHLCGSHLVEALYLVCGERGFFYTDap(G |
| | Dap(DHBA)A₀, | BE5)PT (SEQ ID NO: 124) |
| | Dap(GBE5)B28, | GdDap(DHBA)GIVEQCCTSICSLYQLENYCN |
| | ProB29 | (SEQ ID NO: 130) |
| | GlyA₀, D- | FVNQHLCGSHLVEALYLVCGERGFFYT[Dap( |
| | Dap(DHBA)A1, | GBE5)]PT (SEQ ID NO: 124) |
| | Dap(GBE2)B28, | G[dDap(DHBA)]IVEQCCTSICSLYQLENYCN |
| | ProB29 | (SEQ ID NO: 129) |
| | GA₋₁, | FVNQHLCGSHLVEALYLVCGERGFFYTO(GB |
| | Orn(DHBA)A₀, | E1)]PT (SEQ ID NO: 128) |
| | Orn(GBE1)B28, | G[O(DHBA)]GIVEQCCTSICSLYQLENYCN |
| | ProB29 | (SEQ ID NO 131) |
| | GlyA₀, D- | FVNQHLCGSHLVEALYLVCGERGFFYTO(GB |
| | Orn(DHBA)A1, | E1)]PT (SEQ ID NO: 128) |
| | Orn(GBE1)B28, | G[dO(DHBA)]IVEQCCTSICSLYQLENYCN |
| | ProB29 | (SEQ ID NO: 132) |
| | GlyA₋₁, | FVNQHLCGSHLVEALYLVCGERGFFYTDap(G |
| | Orn(DHBA)A₀, | BE2)PT (SEQ ID NO: 124) |
| | Dap(GBE2)B28, | G[O(DHBA)]GIVEQCCTSICSLYQLENYCN |
| | ProB29 | (SEQ ID NO: 131) |
| | GA₀, D- | FVNQHLCGSHLVEALYLVCGERGFFYTDap(G |
| | Orn(DHBA)A1, | BE2)PT (SEQ ID NO: 124) |
| | Dap(GBE2)B28, | G[dO(DHBA)]IVEQCCTSICSLYQLENYCN |
| | ProB29 | (SEQ ID NO: 132) |
| | GlyA₋₁, [DOPA]A₀, | FVNQHLCGSHLVEALYLVCGERGFFYTO(GB |
| | Orn(GBE1)B28, | E1)]PT (SEQ ID NO: 128) |
| | ProB29 | G[DOPA]GIVEQCCTSICSLYQLENYCN (SEQ |
| | | ID NO: 133) |
| | N(APD)A₋₁, | FVNQHLCGSHLVEALYLVCGERGFFYTO(GB |
| | [DOPA]A₀, | E1)]PT (SEQ ID NO: 128) |
| | Orn(GBE1)B28, | N(APD)[DOPA]GIVEQCCTSICSLYQLENYCN |
| | ProB29 | (SEQ ID NO: 134) |
| | [DOPA] D- | FVNQHLCGSHLVEALYLVCGERGFFYTO(GB |
| | Asn(APD), | E1)]PT (SEQ ID NO: 128) |
| | Orn(GBE1)B28, | [DOPA]dN(APD)IVEQCCTSICSLYQLENYCN |
| | ProB29 | (SEQ ID NO: 135) |
| | A-Chain GBEs, | |
| | (GBE)B28 | |
| | Analogues | Sequence |
| | A-Chain GBE DOIS | Sequence |
| | GlyA₀, D-Cys(GBE1- | FVNQHLCGSHLVEALYLVCGER (SEQ ID NO: |
| | C)A1-DOI | 86)_GdC(GBE1-C)IVEQCCTSICSLYQLENYCN |
| | | (SEQ ID NO: 93) |
| | GlyA₀, D-Cys(GBE2- | FVNQHLCGSHLVEALYLVCGER (SEQ ID NO: |
| | C)A1-DOI | 86) _GdC(GBE2-C) |
| | | IVEQCCTSICSLYQLENYCN (SEQ ID NO: |
| | | 93) |
| | GlyA₋₁, Cys(GBE1- | FVNQHLCGSHLVEALYLVCGERG(SEQ ID |
| | C)A₀-DOI | NO: 86)_GC(GBE1- |
| | | C)GIVEQCCTSICSLYQLENYCN (SEQ ID NO: |
| | | 92) |
| | GlyA₋₁, | FVNQHLCGSHLVEALYLVCGER (SEQ ID NO: |
| | Dap(GBE1)A₀-DOI | 86)_GDap(GBE1)GIVEQCCTSICSLYQLENYCN |
| | | (SEQ ID NO: 137) |
| | GlyA₋₁, | FVNQHLCGSHLVEALYLVCGER (SEQ ID NO: |
| | Dap(GBE2)A₀-DOI | 86) |
| | | _GDap(GBE2)GIVEQCCTSICSLYQLENYCN |
| | | (SEQ ID NO: 137) |
| | GlyA₋₁, | FVNQHLCGSHLVEALYLVCGER (SEQ ID NO: |
| | Dap(GBE4)A₀-DOI | 86)_GDap(GBE4)GIVEQCCTSICSLYQLENYC |
| | | N SEQ ID NO: 137) |
| | GlyA₋₁, | FVNQHLCGSHLVEALYLVCGER (SEQ ID NO: |
| | Dap(GBE5)A₀-DOI | 86)_GDap(GBE5)GIVEQCCTSICSLYQLENYCN |
| | | (SEQ ID NO: 137) |
| | GlyA₋₁, | FVNQHLCGSHLVEALYLVCGER (SEQ ID NO: |
| | Dap(GBE5)A₀-DOI | 86)_GDap(GBE5)GIVEQCCTSICSLYQLENYCN |
| | | (SEQ ID NO: 137) |
| | GlyA₀, D- | FVNQHLCGSHLVEALYLVCGER (SEQ ID NO: |
| | Dap(GBE1)A1-DOI | 86)_GDap(GBE1)GIVEQCCTSICSLYQLENYCN |
| | | (SEQ ID NO: 137) |
| | GlyA₀, D- | FVNQHLCGSHLVEALYLVCGER (SEQ ID NO: |
| | Dap(GBE2)A1-DOI | 86)_GDap(GBE2)GIVEQCCTSICSLYQLENYCN |
| | | (SEQ ID NO: 137) |
| | GlyA₀, D- | FVNQHLCGSHLVEALYLVCGER (SEQ ID NO: |
| | Dap(GBE3)A1-DOI | 86)_GDap(GBE3)GIVEQCCTSICSLYQLENYCN |
| | | (SEQ ID NO: 137) |
| | GlyA₀, D- | FVNQHLCGSHLVEALYLVCGER (SEQ ID NO: |
| | Dap(GBE4)A1-DOI | 86)_GDap(GBE4)GIVEQCCTSICSLYQLENYCN |
| | | (SEQ ID NO: 137) |
| | GlyA₀, D- | FVNQHLCGSHLVEALYLVCGER (SEQ ID NO: |
| | Dap(GBE5)A1-DOI | 86)_GDap(GBE5)GIVEQCCTSICSLYQLENYCN |
| | | (SEQ ID NO: 137) |
| | GlyA₀, D- | FVNQHLCGSHLVEALYLVCGER (SEQ ID NO: |
| | Dap(GBE6)A1-DOI | 86)_GDap(GBE6)GIVEQCCTSICSLYQLENYC |
| | | N (SEQ ID NO: 137) |
| | GlyA₀, D- | FVNQHLCGSHLVEALYLVCGER (SEQ ID NO: |
| | Orn(GBE2)A1-DOI | 86)_GdO(GBE2)IVEQCCTSICSLYQLENYCN |
| | | (SEQ ID NO: 122) |
| | GlyA₋₁, | FVNQHLCGSHLVEALYLVCGER (SEQ ID NO: |
| | Orn(GBE3)A₀-DOI | 86)_GO(GBE3)GIVEQCCTSICSLYQLENYCN |
| | | (SEQ ID NO: 123) |
| | A-Chain DHBA | |
| | DOIs | |
| | GlyA₀, D- | FVNQHLCGSHLVEALYLVCGERG (SEQ ID |
| | Dap(DHBA)A1-DOI | NO: |
| | | 87)_GdDap(DHBA)IVEQCCTSICSLYQLENYCN |
| | | (SEQ ID NO: 129) |
| | GlyA₋₁, | FVNQHLCGSHLVEALYLVCGERG (SEQ ID |
| | Dap(DHBA) A₀-DOI | NO: |
| | | 87)_GDap(DHBA)GIVEQCCTSICSLYQLENYC |
| | | N (SEQ ID NO: 138) |
| | GlyA₋₁, | FVNQHLCGSHLVEALYLVCGERG (SEQ ID |
| | Orn(DHBA)A₀-DOI | NO: |
| | | 87)_GO(DHBA)GIVEQCCTSICSLYQLENYCN |
| | | (SEQ ID NO: 139) |
| | GlyA₀, D- | FVNQHLCGSHLVEALYLVCGER (SEQ ID NO: |
| | Orn(DHBA)A1-DOI | 86)_GdO(DHBA)IVEQCCTSICSLYQLENYCN |
| | | (SEQ ID NO: 140) |

The present disclosure relates to polypeptide hormone analogues that contain a glucose-regulated molecular structure or glucose-detachable molecular moiety, designed respectively either (a) to confer glucose-responsive binding to cognate cellular receptors and/or (b) to enable glucose-mediated liberation of an active insulin analogue. More particularly, the present disclosure is directed to insulin analogues that are responsive to blood-glucose concentrations and their use in the treatment of patients and non-human mammals with Type 1 or Type 2 diabetes mellitus by subcutaneous, intraperitoneal or intravenous injection of the insulin analogues disclosed herein.

The insulin analogues of the present invention may also exhibit other enhanced pharmaceutical properties, such as increased thermodynamic stability, augmented resistance to thermal fibrillation above room temperature, decreased mitogenicity, and/or altered pharmacokinetic and pharmacodynamic properties. More particularly, this disclosure relates to insulin analogues that may confer either rapid action (relative to wild-type insulin in its regular soluble formulation), intermediate action (comparable to NPH insulin formulations known in the art) or protracted action (comparable to basal insulins known in the art as exemplified by insulin detemir and insulin glargine) such that the affinity of the said analogues for the insulin receptor is higher when dissolved in a solution containing glucose at a concentration above the physiological range (>140 mg/dL; hyperglycemia) than when dissolved in a solution containing glucose at a concentration below the physiological range (<80 mg/dL; hypoglycemia).

In accordance with one embodiment an insulin analogue is provided comprising an A chain modified by a glucose-binding element at or near its N terminus and a variant B chain comprising a diol group at the C terminus of the B chain such that the polypeptide chain ends with a hydroxyl group rather than with a carboxylate group. Reduced or absent activity is associated with formation of a covalent bond between the unique diol moiety in the B chain and a second molecular entity located at the N-terminus of the A chain and that contains a glucose-binding element. Displacement of the B chain diol from the A-chain-linked glucose-binding element by glucose would lead to detachment of the tethered molecular entity, which in turn enables high-affinity receptor binding. In the absence of glucose the C-terminus diols remain bound to the A-chain-linked glucose-binding element and the insulin analogue remains inactive.

In accordance with one embodiment the modified B chain may contain a broad molecular diversity of diol-containing moieties (or adducts containing an α-hydroxycarboxylate group as an alternative binding motif that might bind to a glucose-binding element), whether a saccharide or a non-saccharide reagent. Possibilities include an N-linked or O-linked saccharide or any organic moiety of similar molecular mass that contains a diol function that mimics the diol function of a monosaccharide and hence confers reversible PBA-binding activity (or adducts containing an α-hydroxycarboxylate group as an alternative PBA-binding function; PBA in the present invention may equivalently be substituted by other boron-containing diol-binding elements as known in the art to bind glucose). Such non-saccharide diol-containing organic compounds span a broad range of chemical classes, including acids, alcohols, thiol reagents containing aromatic and non-aromatic scaffolds; adducts containing an α-hydroxycarboxylate group may provide an alternative function able to bind PBA or other boron-containing diol-binding elements able to bind glucose. Convenient modes of attachment to the B chain also span a broad range of linkages in addition to the above N-linked and O-linked saccharide derivatives described above; these additional modes of attachment include (i) the side-chain amino function of Lysine, ornithine, diamino-butyric acid, diaminopropionic acid (with main-chain chirality L or D) and (ii) the side-chain thiol function of Cysteine or homocysteine (with main-chain chirality L or D). A preferred embodiment at sites of native aromatic acids (positions B16, B25 and B26) is provided by L-Dopa.

The molecular purpose of the diol-modified B chain is to form an intramolecular bond or bonds with the A-chain-attached glucose-binding element such that the conformation of insulin is “closed” and so impaired in binding to the insulin receptor. Use of a main-chain-directed diol recapitulates the inactive structure of a single-chain insulin analogue. We envision that at high glucose concentrations, the diol-glucose-binding element bond or bonds will be broken due to competitive binding of the glucose to the glucose-binding element. Preferred embodiments contain two or more diol groups in an effort to introduce cooperativity. The main-chain element can be via substitution of the C-terminal carboxylate by a hydroxyl group together with an appropriately positioned side-chain hydroxyl group and/or via a moiety attached to the main-chain nitrogen atom. The analogues of the present invention may optionally contain an additional saccharide-binding element attached to residue B1 as a mechanism intended to provide glucose-sensitive binding of the insulin analogue to surface lectins in the subcutaneous depot. In addition, the analogues of the present invention may optionally contain substitutions known in the art to confer rapid action (such as Asp^{B28}, a substitution found in insulin aspart (the active component of Novolog®); [Lys^{B28}, Pro^{B29}], pairwise substitutions found in insulin lispro (the active component of Humalog®); Glu^{B29} or the combination [Lys^{B3}, Glu^{B29}] as the latter is found in insulin glulisine (the active component of Apridra®), or modifications at position B24 associated with accelerated disassembly of the insulin hexamer (e.g., substitution of Phe^{B24} by Cyclohexanylalanine or by a derivative of Phenylalanine containing a single halogen substitution within the aromatic ring). Alternatively, the analogues of the present invention may optionally contain modifications known in the art to confer protracted action, such as modification of the ε-amino group of Lys^{B29} by an acyl chain or acyl-glutamic acid adduct as respectively illustrated by insulin detemir (the active component of Levemir®) and insulin degludec (the active component of Tresiba®); or contain basic amino-acid substitutions or basic chain extensions designed to shift the isoelectric point (pI) to near neutrality as exemplified by the Arg^{B31}-Arg^{B32} extension of insulin glargine (the active component of Lantus®). Analogues of the present invention designed to exhibit such a shifted pI may also contain a substitution of Asn^{A21}, such as by Glycine, Alanine or Serine. Analogues of the present invention may optionally also contain non-beta-branched amino-acid substitutions of Thr^{A8} associated with increased affinity for the insulin receptor and/or increased thermodynamic stability as may be introduced to mitigate deleterious effects of the primary two above design elements (a phenylboronic acid derivative at or near the N-terminus of the A chain and one or more saccharide derivatives at or near the C-terminus of the B chain) on receptor-binding affinity and/or thermodynamic stability. Examples of such A8 substitutions known in the art are His^{A8}, Lys^{A8}, Arg^{A8}, and Glu^{A8}.

The insulin analogues of the present invention may exhibit an isoelectric point (pI) in the range 4.0-6.0 and thereby be amenable to pharmaceutical formulation in the pH range 6.8-7.8; alternatively, the analogues of the present invention may exhibit an isoelectric point in the range 6.8-7.8 and thereby be amenable to pharmaceutical formulation in the pH range 4.0-4.2. The latter conditions are known in the art to lead to isoelectric precipitation of such a pI-shifted insulin analogue in the subcutaneous depot as a mechanism of protracted action. An example of such a pI-shifted insulin analogue is provided by insulin glargine, in which a basic two-residue extension of the B chain (Arg^{B31}-Arg^{B32}) shifts the pI to near-neutrality and thus enables prolonged pharmacokinetic absorption from the subcutaneous depot. In general the pI of an insulin analogue may be modified through the addition of basic or acidic chain extensions, through the substitution of basic residues by neutral or acidic residues, and through the substitution of acidic residues by neutral or basic residues; in this context we define acidic residues as Aspartic Acid and Glutamic Acid, and we define basic residues as Arginine, Lysine, and under some circumstances, Histidine. We further define a “neutral” residue in relation to the net charge of the side chain at neutral pH.

It is an additional aspect of the present invention that absolute in vitro affinities of the insulin analogue for insulin receptor (isoforms IR-A and IR-B) are in the range 5-100% relative to wild-type human insulin and so unlikely to exhibit prolonged residence times in the hormone-receptor complex; such prolonged residence times are believed to be associated with enhanced risk of carcinogenesis in mammals or more rapid growth of cancer cell lines in culture. It is yet an additional aspect of the present invention that absolute in vitro affinities of the insulin analogue for the Type 1 insulin-like growth factor receptor (IGF-1R) are in the range 5-100% relative to wild-type human insulin and so unlikely either to exhibit prolonged residence times in the hormone/IGF-1R complex or to mediate IGF-1R-related mitogenesis in excess of that mediated by wild-type human insulin.

The insulin analogues of the present invention consist of two polypeptide chains that contain a novel modifications in the B chain such that the analogue, in the absence of glucose or other exogenous saccharide, contains covalent bonds between the side-chain diol in the B chain and a molecular entity containing PBA, a halogen-derivative of PBA, or any boron-containing diol-binding element able to bind glucose. The latter entity may be a C-terminal extension of the B chain or be a separate molecule prior to formation of the diol-PBA bonds.

Table 2 presents diol- or α-hydroxycarboxylate-containing precursors.

TABLE 2

| | |
|---|---|
| 1,3-benzenedimethanol | salicylhydroxamic acid |
| mannitol | catechol |
| fructose | cis-1,2-cyclopentanediol |
| sorbitol | cyclohexane-1,2-diol |
| Tris base | 1,2-dihydroxybenzene |
| Fmoc-3,4-dihydroxy-L-phenylalanine | 2,2,4,4-tetramethyl-1,3- |
| 2-(acetoxymethyl)-4-iodobutyl acetate | cyclobutanediol |
| 1(1R,2S,3R,5R)-3-amino-5- | butylboronic acid |
| (hydroxymethyl)-1,2-cyclopentanediol | isosorbide |
| hydrochloride | N,N-dimethylsphingosine |
| 2-(N-Fmoc-4-aminobutyl)-1,3- | sphingosine (2-amino-4- |
| propanediol | octadecene-1,3-diol) |
| 2-(4-aminobutyl)-1,3-propanediol | tartaric acid |
| 3-amino-1-,2-propandiol | guaifenesin |
| 2-aminopropane-1,3-diol | 5β-Androstane-3α,17α-diol-11- |
| 3-mercaptopropane-1,2-diol | one-17β-carboxylic acid 3-(β-D- |
| 2-amino-4-pentane-1,3-diol | glucuronide) |
| N-acetyl-D-galactosamine | (1S-cis)-3-bromo-3,5- |
| N-acetylquinovosamine | cyclohexadiene-1,2-diol |
| allopumiliotoxin 267A | dihydroxyphenylethylene glycol |
| aminoshikimic acid | |
| atorvastatin | |
| β-D-galactopyranosylamine | |
| cafestol | |
| glafenine | |
| glyceraldehyde | |
| glyceric acid | |
| glycerol 3-phosphate | |
| glycerol monostearate | |
| hydrobromide | |
| 1,2,3,4-tetrahydro isoquinoline-6,7-diol | |
| D-sphingosine | |
| cyclohexane-1,2-diol | |
| cytosine glycol | |
| 4,5-dihydroxy-2,3-pentanedione | |
| dihydroxyphenylethylene glycol | |
| dithioerythritol | |
| dithiothreitol | |
| dropropizine | |
| dyphylline | |
| flavagline FL3 | |
| floctafenine | |
| (3S,4R)-4-methyl-5-hexene-1,3-diol | |
| (3S,4R)-4-Methyl-5-hexene-2,3-diol1,3 | |
| butanediol | |
| erithritol | |

Although we do not wish to be restricted by theory, we envisage that these two design elements form a covalent interaction in the absence of exogenous glucose such that the structure of the hormone is stabilized in a less active conformation.

In accordance with first embodiment 1 an insulin analogue is provided comprising an A chain modified by a glucose-binding element at or near its N terminus and a variant B chain comprising a diol group at the C terminus of the B chain such that the polypeptide chain ends with a hydroxyl group rather than with a carboxylate group.

In accordance with first embodiment 2 an insulin analogue of first embodiment 1 is provided wherein the A chain contains a substitution at position A8 that enhances affinity of the insulin analogue for the insulin receptor, optionally wherein the substitution at position A8 is histidine.

In accordance with first embodiment 3 an insulin analogue of first embodiment 1 or 2 is provided wherein the A chain contains a substitution at position A8 or position A14 that enhances thermodynamic stability of the insulin analogue for the insulin receptor, optionally wherein the substitution at position A8 or A14 is independently selected from the group consisting of HisA8, Lys^{A8}, Arg^{A8}, and Glu^{A8}.

In accordance with first embodiment 4 an insulin analogue of any one of first embodiments 1-3 is provided wherein the A chain contains a substitution at position A21 that protects the insulin analogue from chemical degradation.

In accordance with first embodiment 5 an insulin analogue of any one of first embodiments 1-4 is provided wherein said diol group at the C terminus of the B chain is an aliphatic (1, 2) diol.

In accordance with first embodiment 6 an insulin analogue of any one of first embodiments 1-5 is provided wherein said diol group at the C terminus of the B chain is an aliphatic (1, 3) diol.

In accordance with first embodiment 7 an insulin analogue of any one of first embodiments 1-6 is provided further comprising a modified amino acid at a position 1, 2, 3, or 4 residues N-terminal to the C-terminal amino acid, wherein said modified amino acid is an L or D amino acid comprising a side-chain diol.

In accordance with first embodiment 8 an insulin analogue of any one of first embodiments 1-7 is provided wherein the modified amino acid is
thiol-containing L or D amino acid.

In accordance with first embodiment 9 an insulin analogue of any one of first embodiments 1-8 is provided further comprising an L Dopa at position B26 or an L or D Dopa located at 1-3 residues N-terminal to the C-terminal amino acid.

In accordance with first embodiment 10 an insulin analogue of any one of first embodiments 1-9 is provided wherein said B chain is a truncated B chain lacking residue B30, residues B29-B30, residues B28-B30, residues B27-B30 or residues B26-B30, with a diol group located at the C terminus of the truncated B chain.

In accordance with first embodiment 11 an insulin analogue of any one of first embodiments 1-9 is provided wherein said B chain is extended by one or two amino acids with a diol group located at the C terminus of the extended B chain.

In accordance with first embodiment 12 an insulin analogue of any one of first embodiments 1-9 is provided wherein the B chain is a polypeptide selected from the group consisting of

(SEQ ID NO: 62)
FVKQHLCGSHLVEALYLVCGERGFFYTEKX₃₀,
(SEQ ID NO: 63)
FVNQHLCGSHLVEALYLVCGERGFFYTDKX₃₀,
(SEQ ID NO: 64)
FVNQHLCGSHLVEALYLVCGERGFFYTKPX₃₀,
(SEQ ID NO: 65)
FVNQHLCGSHLVEALYLVCGERGFFYTPKX₃₀
(SEQ ID NO: 66)
FVNQHLCGSHLVEALYLVCGERGFFYTKX₃₀;
(SEQ ID NO: 67)
FVNQHLCGSHLVEALYLVCGERGFFYTPX₂₉X₃₀;
(SEQ ID NO: 68)
FVNQHLCGSHLVEALYLVCGERGFFYTPX₃₀
(SEQ ID NO: 69)
FVNQHLCGSHLVEALYLVCGERGFFYTX₂₉X₃₀
(SEQ ID NO: 70)
FVNQHLCGSHLVEALYLVCGERGFFYTX₃₀
and
(SEQ ID NO: 71)
FVNQHLCGSHLVEALYLVCGERGFFYX₃₀,

X₂₉ is ornithine; and
X₃₀ is a diol bearing amino acid derivative, optionally threoninol. In one first embodiment 13, an insulin analogue of any one of first embodiments 1-9 is provided wherein the B chain is a polypeptide selected from the group consisting of

(SEQ ID NO: 72)
FVNQHLCGSHLVEALYLVCGERGFF[Sar][APD],
(SEQ ID NO: 73)
FVNQHLCGSHLVEALYLVCGERGFF[dA][APD],
and
(SEQ ID NO: 74)
FVNQHLCGSHLVEALYLVCGERGFFYG[APD]

In accordance with first embodiment 14 an insulin analogue of any one of first embodiments 1-9 is provided wherein the B chain is a polypeptide selected from the group consisting of

(SEQ ID NO: 75)
FVNQHLCGSHLVEALYLVCGERGFFYTDKX₃₁X₃₀,
(SEQ ID NO: 76)
FVNQHLCGSHLVEALYLVCGERGFFYTKPX₃₁X₃₀,
(SEQ ID NO: 77)
FVNQHLCGSHLVEALYLVCGERGFFYTPKX₃₁X₃₀,
(SEQ ID NO: 78)
FVNQHLCGSHLVEALYLVCGERGFFYTDKX₃₁X₃₂X₃₀,
(SEQ ID NO: 79)
FVNQHLCGSHLVEALYLVCGERGFFYTKPX₃₁X₃₂X₃₀
and
(SEQ ID NO: 80)
FVNQHLCGSHLVEALYLVCGERGFFYTPKX₃₁X₃₂X₃₀,

wherein
X₃₁ and X₃₂ are independently any amino acid; and
X₃₀ is a diol bearing amino acid derivative, optionally threoninol.

In accordance with first embodiment 15 an insulin analogue of any one of first embodiments 1-14 is provided wherein the A chain is a polypeptide selected from the group consisting of

(SEQ ID NO: 81)
R-GIVEQCCTSICSLYQLENYCN;
and
(SEQ ID NO: 82)
R-GIVEQCCHSICSLYQLENYCN-R53,

R is

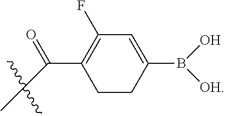 [SMILES: *C(=O)C1=C(F)C=C(B(O)O)CC1]

In accordance with first embodiment 16 a method of preparing an analogue of any one of First embodiments 1-15 is provided by means of trypsin-mediated semi-synthesis wherein (a) any optional A-chain modification (i.e., by a monomeric glucose-binding moiety) is introduced within a des-octapeptide[B23-B30] fragment of insulin or insulin analogue and (b) the diol-containing B-chain modification is introduced within a synthetic peptide of length 5-10 amino-acid residues whose N-terminal residue is Glycine and which upon modification contains no tryptic cleavage site.

In accordance with first embodiment 17 the method of first embodiment 16 is provided wherein the des-octapeptide[B23-B30] fragment of insulin or an insulin analogue is obtained by trypsin digestion of a parent insulin or insulin analogue.

In accordance with first embodiment 18 the method of first embodiment 16 is provided wherein the des-octapeptide[B23-B30] fragment of insulin or insulin analogue is obtained by trypsin digestion of a single-chain polypeptide (such as proinsulin, a proinsulin analogue or a corresponding mini-proinsulin containing a foreshortened or absent C domain) as expressed in Escherichia coli, Saccharomyces cerevisiae, Pichia pastoris or other microbial system for the recombinant expression of proteins.

In accordance with first embodiment 19 the method of first embodiment 16 is provided wherein the des-octapeptide[B23-B30] fragment of insulin or insulin analogue is obtained by trypsin digestion of single-chain polypeptide (such as proinsulin, a proinsulin analogue or a corresponding mini-proinsulin containing a foreshortened or absent C domain) as prepared by solid-phase chemical peptide synthesis, optionally including native fragment-ligation steps.

In accordance with first embodiment 20, a method of treating a diabetic patient is provided wherein the patient is administered a physiologically effective amount of an insulin analogue of any one of first embodiments 1-15, or a physiologically acceptable salt thereof via any standard route of administration.

In accordance with second embodiment 1, an insulin analogue comprising an insulin A chain and an insulin B chain is provided wherein,
said insulin A chain comprises a D-amino acid at position A₁ or A₀ and a glucose-binding element covalently linked at or near the insulin A chain N terminus, optionally at position A₁ or A₀; and
said insulin B chain comprising a diol group at or near the C terminus of the insulin B chain.

In accordance with second embodiment 2 an insulin analogue of second embodiment 1 is provided wherein said glucose-binding element is covalently linked to the side chain of an amino acid in the D-configuration.

In accordance with second embodiment 3 an insulin analogue of second embodiment 1 or 2 is provided further comprising an amino acid added to the N-terminus of the insulin A chain, wherein said N-terminal amino acid is located at position A₀ or A₋₁.

In accordance with second embodiment 4 an insulin analogue of any one of second embodiments 1-3 is provided wherein said N-terminal amino acid is glycine.

In accordance with second embodiment 5 an insulin analogue of any one of second embodiments 1-4 is provided wherein said diol group is linked to the side chain of one of the three most C-terminal amino acids of the insulin B chain, optionally at any of positions B26, B27, B30 or B31, B32 or B33 of a C-terminally extended B chain, optionally at B28, B29 or B30, optionally at B28.

In accordance with second embodiment 6 an insulin analogue of any one of second embodiments 1-5 is provided wherein said diol group is a main chain diol, having the —COOH group of the C-terminal amino acid replaced with —CH₂OH and a side chain bearing an hydroxyl group.

In accordance with second embodiment 7 an insulin analogue of any one of second embodiments 1-6 is provided wherein the A chain contains a substitution at position A8 that enhances affinity of the insulin analogue for the insulin receptor, optionally wherein the substitution at position A8 is histidine.

In accordance with second embodiment 8 an insulin analogue of any one of second embodiments 1-7 is provided wherein the A chain contains a substitution at position A8 or position A14 that enhances thermodynamic stability of the insulin analogue for the insulin receptor, optionally wherein the substitution at position A8 or A14 is independently selected from the group consisting of His, Lys, Arg, and Glu.

In accordance with second embodiment 9 an insulin analogue of any one of second embodiments 1-6 is provided wherein the A chain contains a substitution at position A21 that protects the insulin analogue from chemical degradation.

In accordance with second embodiment 10 an insulin analogue of any one of second embodiments 1-9 is provided wherein said diol group at the C terminus of the B chain is an aliphatic (1, 2) diol.

In accordance with second embodiment 11 an insulin analogue of any one of second embodiments 1-10 is provided wherein said diol group at the C terminus of the B chain is an aliphatic (1, 3) diol.

In accordance with second embodiment 12 an insulin analogue of any one of second embodiments 1-11 is provided further comprising a modified amino acid at a position 1, 2, 3, or 4 residues N-terminal to the C-terminal amino acid of the B chain, wherein said modified amino acid is an L or D amino acid comprising a side-chain diol.

In accordance with second embodiment 13 an insulin analogue of any one of second embodiments 1-12 is provided wherein the modified amino acid is a thiol-containing L or D amino acid.

In accordance with second embodiment 14 an insulin analogue of any one of second embodiments 1-13 is provided further comprising an L Dopa at position B26 or an L or D Dopa located at 1-3 residues N-terminal to the C-terminal amino acid of the B chain.

In accordance with second embodiment 15 an insulin analogue of any one of second embodiments 1-14 is provided wherein said B chain is a truncated B chain lacking residue B30, residues B29-B30, residues B28-B30, residues B27-B30 or residues B26-B30, with a diol group located at the C terminus of the truncated B chain.

In accordance with second embodiment 16 an insulin analogue of any one of second embodiments 1-15 is provided wherein said B chain is extended by one or two amino acids with a diol group located at the C terminus of the extended B chain.

In accordance with second embodiment 17 an insulin analogue of any one of second embodiments 1-16 is provided wherein
the insulin A chain is a polypeptide selected from the group consisting of
X₋₁X₀X₁IVEQX₆CX₈SIX₁₁SLYQLENYCN (SEQ ID NO: 4) and
X₀X₁IVEQX₆CX₈SIX₁₁SLYQLENYCN (SEQ ID NO: 5), wherein
   X₋₁ is any amino acid, optionally X₋₁ is Gly;
   X₀ is any amino acid or a modified amino acid comprising a glucose-binding element linked to its side chain; and
   X₁ is any amino acid or a modified amino acid comprising a glucose-binding element linked to its side chain;
   X₆ and X₁₁ are each Cys or selenocysteine; and
   X₈ is Thr, His, Lys, Arg, or Glu;
   further wherein at least one of X₋₁, X₀ and X₁ is in the D conformation; and
the insulin B chain is a polypeptide selected from the group consisting of
FVX₂₄QHLCGSHLVEALYLVCGERGFFYTX₄₉X₅₀X₅₁ (SEQ ID NO: 6), and
FV X₂₄QHLCGSHLVEALYLVCGERGFFYTX₅₁ (SEQ ID NO: 7), wherein
   X₂₄ is Lys or Asn;
   X₄₉ is Glu Asp, Lys, Pro, or a modified amino acid comprising a diol bearing moiety linked to the side chain of the amino acid;
   X₅₀ is Orn, Lys, Pro, or a modified amino acid comprising a diol bearing moiety linked to the side chain of the amino acid; and
   X₅₁ is Thr or a diol bearing amino acid.

In accordance with second embodiment 18 an insulin analogue of any one of second embodiments 1-17 is provided wherein
the insulin B chain is a polypeptide comprising the sequence of

(SEQ ID NO: 6)
FVX₂₄QHLCGSHLVEALYLVCGERGFFYTX₄₉X₅₀X₅₁,

wherein
X₂₄ is Lys or Asn;
X₄₉ is a modified amino acid comprising a diol bearing moiety linked to the side chain of the amino acid;
X₅₀ is Orn, Lys or Pro; and
X₅₁ is Thr or a diol bearing amino acid.

In accordance with second embodiment 19 an insulin analogue of any one of second embodiments 1-18 is provided wherein
X₂₄ is Lys or Asn;
X₄₉ is a modified amino acid comprising a diol bearing moiety linked to the side chain of the amino acid;
X₅₀ is Orn, Lys or Pro; and
X₅₁ is diol bearing amino acid, optionally wherein the diol bearing amino acid comprises a diol bearing moiety linked to the side chain of the amino acid or the diol bearing amino acid is a main chain.

In accordance with second embodiment 20 an insulin analogue of any one of second embodiments 1-19 is provided wherein
X₂₄ is Lys or Asn;
X₄₉ is a modified amino acid comprising a diol bearing moiety linked to the side chain of the amino acid;
X₅₀ is Pro; and
X₅₁ is Thr.

In accordance with second embodiment 21 an insulin analogue of any one of second embodiments 1-20 is provided wherein
the insulin A chain is a polypeptide comprising the sequence of
X₀X₁IVEQX₆CX₈SIX₁₁SLYQLENYCN (SEQ ID NO: 5), wherein
   X₀ is any amino acid, optional Gly; and
   X₁ is a modified D-amino acid comprising a glucose-binding element linked to its side chain;
   X₆ and X₁₁ are each Cys or selenocysteine; and
   X₈ is Thr, His, Lys, Arg, or Glu.

In accordance with second embodiment 22 an insulin analogue of any one of second embodiments 1-21 is provided wherein the diol bearing amino acid is selected from L-Cys, L-Lys, D-Lys, L-Orn, D-Orn, L-Dab, and D-Dab further modified to comprise a diol bearing moiety linked to the side chain of the amino acid or further modified to replace the carboxyl group with CH₂OH.

In accordance with second embodiment 23 an insulin analog of any one of second embodiments 1-22 is provided wherein diol bearing amino acid comprises a diol moiety having the structure of

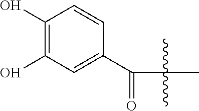 [SMILES: *C(=O)c1ccc(O)c(O)c1]

linked to the side chain or backbone amide of the insulin B chain peptide.

In accordance with second embodiment 24 an insulin analog of any one of second embodiments 1-23 is provided wherein the glucose-binding elements are linked to the A chain and comprise a complex having the general structure

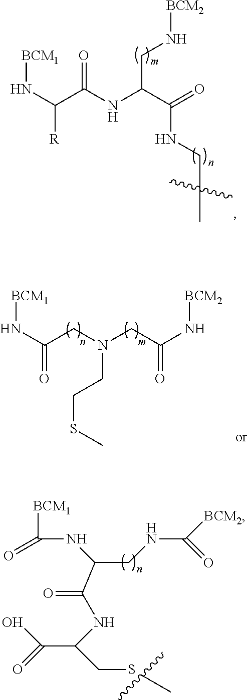

wherein BCM1 and BCM2 are boron-containing moieties; n and m are independently an integer selected from the range of 1 to 3; and R is any amino acid side chain of the L or D configurations standard 20 essential amino acids. In one embodiment R is selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂+)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O. In one embodiment R is H, C₁-C₆ alkyl, (C₁-C₄ alkyl)C(O)NH₂, (C₁-C₄ alkyl)OH, (C₁-C₄ alkyl)CH₃OH, (C₁-C₄ alkyl)S, (C₁-C₄ alkyl)SCH₃, (C₁-C₄ alkyl)COOH and (C₁-C₄ alkyl)NH₂.

In accordance with second embodiment 25 an insulin analogue of any one of second embodiments 1-22 is provided wherein the insulin B chain is a polypeptide selected from the group consisting of
FVKQHLCGSHLVEALYLVCGERGFFYTEKX₅₁ (SEQ ID NO: 62),
FVNQHLCGSHLVEALYLVCGERGFFYTDKX₅₁ (SEQ ID NO: 63),
FVNQHLCGSHLVEALYLVCGERGFFYTKPX₅₁ (SEQ ID NO: 64),
FVNQHLCGSHLVEALYLVCGERGFFYTPKX₅₁ (SEQ ID NO: 65)
FVNQHLCGSHLVEALYLVCGERGFFYTKPX₅₁ (SEQ ID NO: 66);
FVNQHLCGSHLVEALYLVCGERGFFYTPX₅₀X₅₁ (SEQ ID NO: 67);
FVNQHLCGSHLVEALYLVCGERGFFYTPX₅₁ (SEQ ID NO: 68)
FVNQHLCGSHLVEALYLVCGERGFFYTX₅₀X₅₁ (SEQ ID NO: 69)
FVNQHLCGSHLVEALYLVCGERGFFYTX₅₁ (SEQ ID NO: 70) and
FVNQHLCGSHLVEALYLVCGERGFFYX₅₁ (SEQ ID NO: 71), wherein
   X₅₀ is Om or Lys; and
   X₅₁ is a diol bearing amino acid derivative, optionally threoninol or 3-amino-1,2-propandiol.

In accordance with second embodiment 24 an insulin analogue of any one of second embodiments 1-23 is provided wherein the B chain is a polypeptide selected from the group consisting of
FVNQHLCGSHLVEALYLVCGERGFF[Sar][APD] (SEQ ID NO: 72),
FVNQHLCGSHLVEALYLVCGERGFF[dA][APD] (SEQ ID NO: 73), and
FVNQHLCGSHLVEALYLVCGERGFFYG[APD] (SEQ ID NO: 74),
   wherein APD is 3-amino-1,2-propandiol.

In accordance with second embodiment 25 and insulin analogue of any one of second embodiments 1-24 is provided wherein the diol-bearing amino acids comprises a natural or unnatural amino acids whose side chains are modified by linkage of a diol moiety to an attachment point located on the amino acid side chain, optionally wherein the amino acid is selected from the group consisting of Cysteine Homocysteine, Lysine, Ornithine (Orn), Diaminobutyric Acid (Dab) and Diaminoproprionic acid (Dap), each in either the L- or D configuration with the covalent linkage of a diol moiety, exemplified (but not restricted to) those listed in Table 2, to the amino acid side chain; and the glucose binding element is a compound selected from those listed in FIGS. 10A-11C.

1. An insulin analogue comprising an insulin A chain and an insulin B chain,
said insulin A chain comprising either (a) a D-amino acid at position A1, (b) an L- or D-amino acid at A₀ or (c) an L- or D amino acid at A₋₁ to which one or more glucose-binding elements is covalently linked, where residues A₀ and A₋₁ represent optional N-terminal extensions of the A chain; and said insulin B chain contains one or more diol groups at or near the C terminus of the insulin B chain, optionally with the proviso that at least one of positions A₀ and A1 is present and is a D amino acid.

2. The insulin analogue of claim 1 wherein said glucose-binding element contains two or more boron atoms and is covalently linked to either (a) the side chain of an amino acid at positions A₋₁, A₀ or A₁ and/or (b) to the α-amino group of residues A₋₁, A₀ or A₁.

3. The insulin analogue of claim 1 comprising 1 or 2 amino acids added to the N-terminus of the native insulin A chain.

4. The insulin analogue of claim 3 wherein the N-terminal amino acid of said insulin A chain is glycine.

5. The insulin analogue of claim 3 wherein said diol group is linked to a side chain of residues B26, B27, B28, B29, B30 or to C-terminal extension B31 or B31-B32.

6. The insulin analogue of claim 3 wherein said diol group is a main chain diol, having the —COOH group of the C-terminal amino acid replaced with —CH₂OH, optionally in combination with a side-chain modification of residues B26, B27, B28, B29, B30 or C-terminal extension B31 or B31-B32 residue with a diol bearing moiety.

7. The insulin analogue of claim 1 wherein the A chain contains a substitution at position A8 that enhances affinity of the insulin analogue for the insulin receptor, optionally wherein the A8 substitution is a nitrogen-containing amino acid selected from the group Lysine, Histidine or Glutamine.

8. The insulin analogue of claim 1 wherein the A chain contains a substitution at position A8 or position A14 that enhances thermodynamic stability of the insulin analogue for the insulin receptor, optionally wherein the substitution at position A8 or A14 is independently selected from the group consisting of His, Lys, Arg, and Glu.

9. The insulin analogue of claim 1 wherein the A chain contains a substitution at position A21 that protects the insulin analogue from chemical degradation.

10. The insulin analogue of claim 3 wherein said diol group at the C terminus of the B chain is an aliphatic (1, 2) diol.

11. The insulin analogue of claim 3 wherein said diol group at the C terminus of the B chain is an aliphatic (1, 3) diol.

12. (canceled)

13. (canceled)

14. The insulin analogue of claim 1 where said B chain is a truncated B chain lacking residue B30, residues B29-B30, residues B28-B30, residues B27-B30 or residues B26-B30, with a diol group located at the C terminus of the truncated B chain.

15. The insulin analogue of claim 1 where said B chain is extended by one or two amino acids with a diol group located at the C terminus of the extended B chain.

16. The insulin analogue of claim 1 wherein
the insulin A chain is a polypeptide selected from the group consisting of
X₋₁X₀X₁IVEQX₆CX₈SIXIISLYQLENYCN (SEQ ID NO: 4),
X₀X₁IVEQX₆CX₈SIXIISLYQLENYCN (SEQ ID NO: 5), and
X₇₀IVEQX₆CX₈SIX₁₁SLYQLENYCN (SEQ ID NO: 83) wherein
X₋₁ is any amino acid, optionally wherein X₋₁ is Gly;
X₀ is any amino acid or a modified amino acid comprising a glucose-binding element linked to its side chain;
X₁ is any amino acid or a modified amino acid comprising a glucose-binding element linked to its side chain, optionally wherein X₋₁ is Gly;
X₆ and X₁₁ are each Cys or selenocysteine; and
X₈ is Thr, His, Lys, Arg, or Glu;
X₇₀ is a D-amino acid comprising a glucose-binding element linked to its side chain;
further wherein at least one of X₋₁, X₀ and X₁ is in the D conformation and
the insulin B chain is a polypeptide selected from the group consisting of
FVX₂₄QHLCGSHLVEALYLVCGERGFFYTX₄₉X₅₀X₅₁ (SEQ ID NO: 6), and
FVX₂₄QHLCGSHLVEALYLVCGERGFFYTX₅₁ (SEQ ID NO: 7), wherein
X₂₄ is Lys or Asn;
X₄₉ is Orn, Glu Asp, Lys, Pro, or a modified amino acid comprising a diol bearing moiety linked to the side chain of the amino acid;
X₅₀ is Orn, Lys, Pro, or a modified amino acid comprising a diol bearing moiety linked to the side chain of the amino acid; and
X₅₁ is Thr or a diol-bearing amino acid.

17.-21. (canceled)

22. An insulin analogue of claim 1 wherein the B chain is a polypeptide selected from the group consisting of
FVNQHLCGSHLVEALYLVCGERGFF[Sar][APD] (SEQ ID NO: 37),
FVNQHLCGSHLVEALYLVCGERGFF[dA][APD] (SEQ ID NO: 38), and
FVNQHLCGSHLVEALYLVCGERGFFYG[APD] (SEQ ID NO: 39), wherein APD is 3-amino-1,2-propandiol.

23. An insulin analogue of claim 1 wherein the glucose-binding element contains two or more boron atoms, optionally containing phenyl-boronic acid or a halogen-modified phenyl boronic acid, such that one or more moieties is selected from the following:

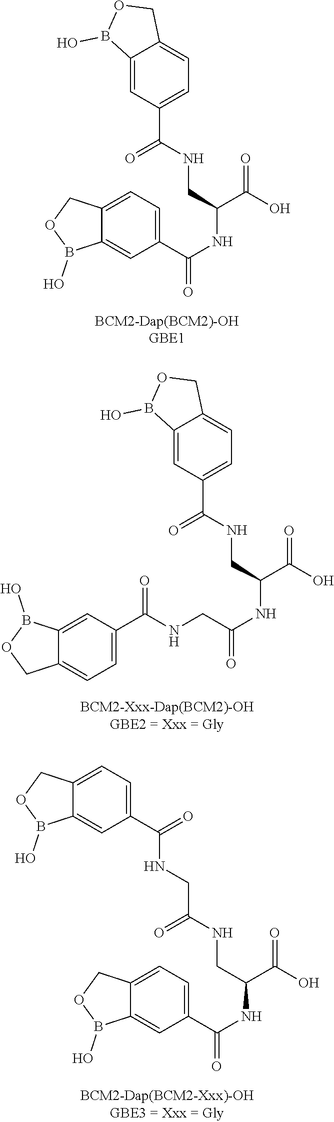 [SMILES: O=C(NC[C@H](NC(=O)c1ccc2c(c1)B(O)OC2)C(=O)O)c1ccc2c(c1)B(O)OC2 ; O=C(CNC(=O)c1ccc2c(c1)B(O)OC2)N[C@@H](CNC(=O)c1ccc2c(c1)B(O)OC2)C(=O)O ; O=C(CNC(=O)c1ccc2c(c1)B(O)OC2)NC[C@H](NC(=O)c1ccc2c(c1)B(O)OC2)C(=O)O]

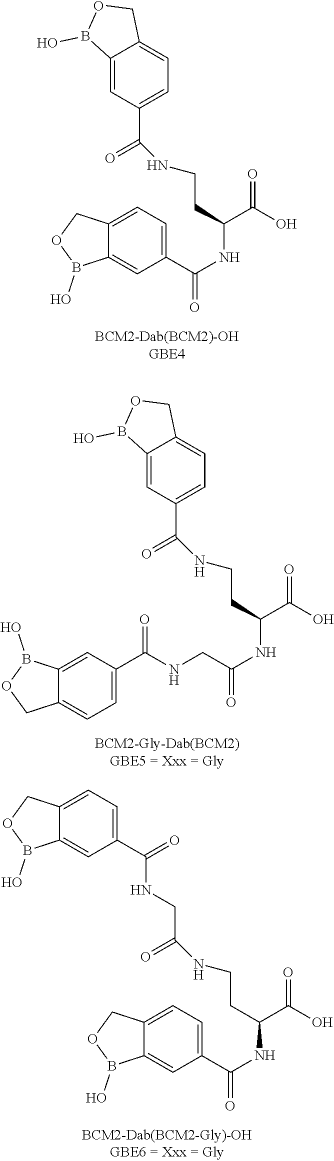 [SMILES: O=C(NCC[C@H](NC(=O)c1ccc2c(c1)B(O)OC2)C(=O)O)c1ccc2c(c1)B(O)OC2 ; O=C(CNC(=O)c1ccc2c(c1)B(O)OC2)N[C@@H](CCNC(=O)c1ccc2c(c1)B(O)OC2)C(=O)O ; O=C(CNC(=O)c1ccc2c(c1)B(O)OC2)NCC[C@H](NC(=O)c1ccc2c(c1)B(O)OC2)C(=O)O]

24. An insulin analogue of claim 1 wherein the insulin analogue is a “flipped” insulin analogue comprising a boron-containing glucose-binding element linked at or near the C terminus of the B chain while one or more diol groups are linked at or near the N terminus of the A chain, defined as residues A₋₁, A₀ or A1.

25. A method of preparing an analogue by means of trypsin-mediated semi-synthesis comprising (a) introducing an optional A-chain modification within a des-octapeptide[B23-B30] fragment of insulin or an insulin analogue and (b) introducing a diol-containing B-chain modification within a synthetic peptide of length 5-10 amino-acid residues whose N-terminal residue is Glycine and which upon modification contains no tryptic cleavage site.

26. The method of claim 25 wherein the des-octapeptide[B23-B30] fragment of insulin or an insulin analogue is obtained by trypsin digestion of a parent insulin or insulin analogue.

27. A method of claim 25 wherein the des-octapeptide[B23-B30] fragment of insulin or insulin analogue is obtained by trypsin digestion of a single-chain polypeptide (such as proinsulin, a proinsulin analogue or a corresponding mini-proinsulin containing a foreshortened or absent C domain) as expressed in Escherichia coli, Saccharomyces cerevisiae, Pichia pastoris or other microbial system for the recombinant expression of proteins.

28. (canceled)

29. (canceled)
